SEQUENCE LISTING (126 sequences)

SEQ ID NO: 1 - protein, 514 aa, Cauliflower mosaic virus
SKTQQPEPVNISTNKIENPLEEIAILSEGRRLSEEKLFITQQRMQKTEELLEKVCSENPLDPNKTKQWMKASIKLSDPSKAIKVKPMKYSPMDREEFDKQIKELLDLKVIKPSKSPHMAPAFLVNNEAENGRGNKRMVVNYKAMNKATVGDAYNLPNKDELLTLIRGKKIFSSFDCKSGFWQVLLDQESRPLTAFTCPQGHYEWNVVPFGLKQAPSIFQRHMDEAFRVFRKFCCVYVDDIVVFSNNEEDHLLHVAMILQKCNQHGIILSKKKAQLFKKKINFLGLEIDEGTHKPQGHILEHINKFPDTLEDKKQLQRFLGILTYASDYIPNLAQMRQPLQAKLKENVPWKWTKEDTLYMQKVKKNLQGFPPLHHPLPEEKLIIETDASDDYWGGMLKAIKINEGTNTELICRYRSGSFKAAERNYHSNDKETLAVINTIKKFSIYLTPVHFLIRTDNTHFKSFVNLNYKGDSKLGRNIRWQAWLSHYSFDVEHIKGTDNHFADFLSREFNKVNS

SEQ ID NO: 2 - protein, 498 aa, Dahlia Mosaic virus
SNTQPLPGTNITKEAVEDESNFIIDISRYQEIQNLLKKVCSENPIDPAKSKAWMKASIKLADPKSVVRVKPMVYSPEDRKEFEIQIKELLDLKVIEPSKSQHMSPAFLVEKEAEKGRGKKRMVVNYKKLNEVTIGDSHNLPNMQELITLLRGKTIFSSFDCKSGFWQVFLDQESQKLTAFTCPQGHFQWRVVPFGLKQAPSIFQRHMQNALRGLEEFCLVYVDDIIVFSDKEEEHYTHVLKVLKRIESLGIILSEKKANLFKEKINFLGLEIDRGTHTPQNHILEHLHNFPDRLEDKKQLQRFLGVLTYADSYIPKLAEKRKPLQVKLKKDQVWIWNQSDTDYVKKIKKGLVNFPKLYLPKKEDSLIIETDASDHFWGGVLKAQTTEGNELICRYSSGTFKPAELNYHSNEKELLAVKQVITKFSIYLTPVTFTVRTDNVNLLKRFMNTKITGDSKQGRLIRWQMWLSHYTFNVNHLKGEKNVLADYLTREYQQKRQQ

SEQ ID NO: 3 - protein, 595 aa, Arabidopsis thaliana
AQSLSTPAQSSSSSPSPTTSASSSSTSPTPPSILIHPPPPLAQIVNNNNQAPLNTHSMGTRAKAGIIKPNPKYSLAVSLAAESEPRTAIQALKDERWRNAMGSEINAQIGNHTWDLVPPPPSHVTIVGCRWIFTKKYNSDGSLNRYKARLVAKGYNQRPGLDYAETFSPVIKSTSIRIVLGVAVDRSWPIRQLDVNNAFLQGTLTDDVYMSQPPGFIDKDRPNYVCKLRKALYGLKQAPRAWYVELRNYLLTIGFVNSVSDTSLFVLQRGKSIVYMLVYVDDILITGNDPTLLHNTLDNLSQRFSVKDHEELHYFLGIEAKRVPTGLHLSQRRYILDLLARTNMITAKPVTTPMAPSPKLSLYSGTKLTDPTEYRGIVGSLQYLAFTRPDISYAVNRLSQFMHMPTEEHLQALKRILRYLAGTPNHGIFLKKGNTLSLHAYSDADWAGDKDDYVSTNGYIVYLGHHPISWSSKKQKGVVRSSTEAEYRSVANTSSEMQWICSLLTELGIRLTRPPVIYCDNVGATYLCANPVFHSRMKHIAIDYHFIRNQVQSGALRVVHVSTHDQLADTLTKPLSRTAFQNFASKIGVTRVPPS

SEQ ID NO: 4 - protein, 606 aa, Arabidopsis thaliana
NQNSPLPQSPISSPHIPTPSTSISEPNSPSSSSTSTPPLPPVLPAPPIIQVNAQAPVNTHSMATRAKDGIRKPNQKYSYATSLAANSEPRTAIQAMKDDRWRQAMGSEINAQIGNHTWDLVPPPPPSVTIVGCRWIFTKKFNSDGSLNRYKARLVAKGYNQRPGLDYAETFSPVIKSTSIRIVLGVAVDRSWPIRQLDVNNAFLQGTLTDEVYMSQPPGFVDKDRPDYVCRLRKAIYGLKQAPRAWYVELRTYLLTVGFVNSISDTSLFVLQRGRSIIYMLVYVDDILITGNDTVLLKHTLDALSQRFSVKEHEDLHYFLGIEAKRVPQGLHLSQRRYTLDLLARTNMLTAKPVATPMATSPKLTLHSGTKLPDPTEYRGIVGSLQYLAFTRPDLSYAVNRLSQYMHMPTDDHWNALKRVLRYLAGTPDHGIFLKKGNTLSLHAYSDADWAGDTDDYVSTNGYIVYLGHHPISWSSKKQKGVVRSSTEAEYRSVANTSSELQWICSLLTELGIQLSHPPVIYCDNVGATYLCANPVFHSRMKHIALDYHFIRNQVQSGALRVVHVSTHDQLADTLTKPLSRVAFQNFSRKIGVIKVPPSCGGVLRI

SEQ ID NO: 5 - protein, 600 aa, Oryza sativa
SEMSPFPFHYSRRPRTPHESQPPSCLPSDVPTSTINAQSDDPPVAPLIDDPLVSSPQSATPRYGLRDRSKIMPHPRYMCAAIDGVQEPSTYQEAAAIPEWQLAMTEELAALERTRTWDIVPLPCHAIPITCKWVFKVKTRSDGSIERYKARLVARGFQQEHGRDYDETFAPVAHMTTVRTLVAVAAARSWTISQMDVKNAFLHGDLKEEVYMKPPPGVDAPANYVFRLNRALYGLKQAPRAWFERFSSVVCAAGFSPSDHDPALFIHVSDHGRTLLLLYVDDMLITGDDLEYIAFVKQCLSEQFMMSDLGPLSYFLGIEVTNTEDGYYLSQHRYIDDLITRSGLTDTKTATTPMELHVQLRSTEGVLLEDPSRYRQIVGSLVYLTITRPDIAHIVHVLSQFVSAPTSVHYSHLLRVLRYLRGTASRQLFYARSSQLHLHAYSDSTWASDPMDRRSITGYCIFLGTSLIAWKSKKQTAVSRSSTESELRALATTTSEIVWLRWLLSDLGVECKDPTPLLCDNTGAIQIANDPVKHELTKHIGVDASFTRSHCQQSTISLRYVPSELQVADFFTKAQTREQHRLYILKLNVIDPPNHSPNPP

SEQ ID NO: 6 - protein, 517 aa, Oryza sativa
LKGSCFIATKSDLDEVDTDTVFCYALVCKETLFPIEDTPISLRPPVTNLLQEYADIFPKEVPPGLPPIRGIEHQIDLIPGASLPNRAPYRTNPEETKEIQRQVQELLDKGYVRESLSPCSVPVLLVPKKDGSWRMCVDCRAINNITIRYRHPIPRLDDMLDELSGSLVFSKIDLRSGYHQIRMKLGDEWKTAFKTKFGLYEWLVMPFGLTNAPSTFMRLMNEVLRAFIGRFVVVYFDDILIYSRSIEDHHGHLRAVFDALRDARLFGNLEKCTFCTDRVSFLGYVVTPQGIEVDQAKVEAIHSWPVPTTITQVRSFLGLAGFYRRFVKDFSTIAAPLHELTKRNVTFTWAAAQRNAFDTLKDKLTHAPLLQLPDFNKTFELECDASGIGLGGVLLQEGKPVAYFSEKLSGPSLNYSTYDKELFALVRTLETWQHYLWPKEFVIHSDHESLKHIRSQAKHNRRHAKWVEFIESFPYVIKHKKGKENVIADALSRRYAMLSQLDFKIFGLETIKEQYAH

SEQ ID NO: 7 - protein, 620 aa, Nicotiana tabacum
VRTAADMSEKVKNGIIPNFVTIPSTSNNPTSAESTTDEVSEQGEQPGEVIEQGEQLDEGVEEVEHPTQGEEQHQPLRRSERPRVESRRYPSTEYVLISDDREPESLKEVLSHPEKNQLMKAMQEEMESLQKNGTYKLVELPKGKRPLKCKWVFKLKKDGDCKLVRYKARLVVKGFEQKKGIDFDEIFSPVVKMTSIRTILSLAASLDLEVEQLDVKTAFLHGDLEEEIYMEQPEGFEVAGKKHMVCKLNKSLYGLKQAPRQWYMKFDSFMKSQTYLKTYSDPCVYFKRFSENNFIILLLYVDDMLIVGKDKGLIAKLKGDLSKSFDMKDLGPAQQILGMKIVRERTSRKLWLSQEKYIERVLERFNMKNAKPVSTPLAGHLKLSKKMCPTTVEEKGNMAKVPYSSAVGSLMYAMVCTRPDIAHAVGVVSRFLENPGKEHWEAVKWILRYLRGTTGDCLCFGGSDPILKGYTDADMAGDIDNRKSSTGYLFTFSGGAISWQSKLQKCVALSTTEAEYIAATETGKEMIWLKRFLQELGLHQKEYVVYCDSQSAIDLSKNSMYHARTKHIDVRYHWIREMVDDESLKVLKISTNENPADMLTKVVPRNKFELCKELVGMHSN

SEQ ID NO: 8 - protein, 603 aa, Drosophila melanogaster
SKKRKRDDHLNESKGSGNPNESRESETAEHLKEIGIDNPTKNDGIEIINRRSERLKTKPQISYNEEDNSLNKVVLNAHTIFNDVPNSFDEIQYRDDKSSWEEAINTELNAHKINNTWTITKRPENKNIVDSRWVFSVKYNELGNPIRYKARLVARGFTQKYQIDYEETFAPVARISSFRFILSLVIQYNLKVHQMDVKTAFLNGTLKEEIYMRLPQGISCNSDNVCKLNKAIYGLKQAARCWFEVFEQALKECEFVNSSVDRCIYILDKGNINENIYVLLYVDDVVIATGDMTRMNNFKRYLMEKFRMTDLNEIKHFIGIRIEMQEDKIYLSQSAYVKKILSKFNMENCNAVSTPLPSKINYELLNSDEDCNTPCRSLIGCLMYIMLCTRPDLTTAVNILSRYSSKNNSELWQNLKRVLRYLKGTIDMKLIFKKNLAFENKIIGYVDSDWAGSEIDRKSTTGYLFKMFDFNLICWNTKRQNSVAASSTEAEYMALFEAVREALWLKFLLTSINIKLENPIKIYEDNQGCISIANNPSCHKRAKHIDIKYHFAREQVQNNVICLEYIPTENQLADIFTKPLPAARFVELRDKLGLLQDDQSNAE

SEQ ID NO: 9 - protein, 677 aa, Artificial Sequence
  Moloney Murine Leukemia Virus RTmut_PE2
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 10 - protein, 1954 aa, Artificial
  nCas9-CaMVRT
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSSKTQQPEPVNISTNKIENPLEEIAILSEGRRLSEEKLFITQQRMQKTEELLEKVCSENPLDPNKTKQWMKASIKLSDPSKAIKVKPMKYSPMDREEFDKQIKELLDLKVIKPSKSPHMAPAFLVNNEAENGRGNKRMVVNYKAMNKATVGDAYNLPNKDELLTLIRGKKIFSSFDCKSGFWQVLLDQESRPLTAFTCPQGHYEWNVVPFGLKQAPSIFQRHMDEAFRVFRKFCCVYVDDIVVFSNNEEDHLLHVAMILQKCNQHGIILSKKKAQLFKKKINFLGLEIDEGTHKPQGHILEHINKFPDTLEDKKQLQRFLGILTYASDYIPNLAQMRQPLQAKLKENVPWKWTKEDTLYMQKVKKNLQGFPPLHHPLPEEKLIIETDASDDYWGGMLKAIKINEGTNTELICRYRSGSFKAAERNYHSNDKETLAVINTIKKFSIYLTPVHFLIRTDNTHFKSFVNLNYKGDSKLGRNIRWQAWLSHYSFDVEHIKGTDNHFADFLSREFNKVNSSGGSKRTADGSEFEPKKKRKV

SEQ ID NO: 11 - protein, 2040 aa, Artificial
  nCas9-LullabyRT
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSSEMSPFPFHYSRRPRTPHESQPPSCLPSDVPTSTINAQSDDPPVAPLIDDPLVSSPQSATPRYGLRDRSKIMPHPRYMCAAIDGVQEPSTYQEAAAIPEWQLAMTEELAALERTRTWDIVPLPCHAIPITCKWVFKVKTRSDGSIERYKARLVARGFQQEHGRDYDETFAPVAHMTTVRTLVAVAAARSWTISQMDVKNAFLHGDLKEEVYMKPPPGVDAPANYVFRLNRALYGLKQAPRAWFERFSSVVCAAGFSPSDHDPALFIHVSDHGRTLLLLYVDDMLITGDDLEYIAFVKQCLSEQFMMSDLGPLSYFLGIEVTNTEDGYYLSQHRYIDDLITRSGLTDTKTATTPMELHVQLRSTEGVLLEDPSRYRQIVGSLVYLTITRPDIAHIVHVLSQFVSAPTSVHYSHLLRVLRYLRGTASRQLFYARSSQLHLHAYSDSTWASDPMDRRSITGYCIFLGTSLIAWKSKKQTAVSRSSTESELRALATTTSEIVWLRWLLSDLGVECKDPTPLLCDNTGAIQIANDPVKHELTKHIGVDASFTRSHCQQSTISLRYVPSELQVADFFTKAQTREQHRLYILKLNVIDPPNHSPNPPSGGSKRTADGSEFEPKKKRKV

SEQ ID NO: 12 - protein, 2060 aa, Artificial
  nCas9-TnT1RT
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSVRTAADMSEKVKNGIIPNFVTIPSTSNNPTSAESTTDEVSEQGEQPGEVIEQGEQLDEGVEEVEHPTQGEEQHQPLRRSERPRVESRRYPSTEYVLISDDREPESLKEVLSHPEKNQLMKAMQEEMESLQKNGTYKLVELPKGKRPLKCKWVFKLKKDGDCKLVRYKARLVVKGFEQKKGIDFDEIFSPVVKMTSIRTILSLAASLDLEVEQLDVKTAFLHGDLEEEIYMEQPEGFEVAGKKHMVCKLNKSLYGLKQAPRQWYMKFDSFMKSQTYLKTYSDPCVYFKRFSENNFIILLLYVDDMLIVGKDKGLIAKLKGDLSKSFDMKDLGPAQQILGMKIVRERTSRKLWLSQEKYIERVLERFNMKNAKPVSTPLAGHLKLSKKMCPTTVEEKGNMAKVPYSSAVGSLMYAMVCTRPDIAHAVGVVSRFLENPGKEHWEAVKWILRYLRGTTGDCLCFGGSDPILKGYTDADMAGDIDNRKSSTGYLFTFSGGAISWQSKLQKCVALSTTEAEYIAATETGKEMIWLKRFLQELGLHQKEYVVYCDSQSAIDLSKNSMYHARTKHIDVRYHWIREMVDDESLKVLKISTNENPADMLTKVVPRNKFELCKELVGMHSNSGGSKRTADGSEFEPKKKRKV

SEQ ID NO: 13 - DNA, 1917 bp, Zea mays (var. A188)
atggccaccgccgccaccgcggccgccgcgctcaccggcgccactaccgctacgcccaagtcgaggcgccgagcccaccacttggccacccggcgcgccctcgccgcgcccatcaggtgctcagcgttgtcacgcgccacgccgacggctcccccggccactccgctacgtccgtggggccccaacgagccccgcaagggctccgacatcctcgtcgaggctctcgagcgctgtggcgtccgtgacgtcttcgcctaccccggcggcgcatccatggagatccaccaggcactcacccgctcccccgtcatcgccaaccacctcttccgccacgaacaaggggaggccttcgccgcctccggctacgcgcgctcctcgggccgcgttggcgtctgcatcgccacctccggccccggcgccaccaacctagtctctgcgctcgcagacgcgttgctcgactccgtccccattgtcgccatcacgggacaggtgccgcgacgcatgattggcaccgacgcctttcaggagacgcccatcgtcgaggtcacccgctccatcaccaagcacaactacctggtcctcgacgtcgacgacatcccccgcgtcgtgcaggaggccttcttcctcgcatcctctggtcgcccggggccggtgcttgttgacatccccaaggacatccagcagcagatggcggtgccggcctgggacacgcccatgagtctgcctgggtacatcgcgcgccttcccaagcctcccgcgactgaatttcttgagcaggtgctgcgtcttgttggtgaatcacggcgccctgttctttatgttggcggtggctgtgcagtatcaggtgaggagttgtgccgctttgtggagttgactggaatcccagtcacaactactcttatgggccttggcaacttccccagcgacgacccactgtcactgcgcatgcttggtatgcatggcacagtgtatgcaaattatgcagtggataaggccgatctgttgcttgcatttggtgtgcggtttgatgatcgtgtgacagggaaaattgaggcttttgcaggcagagctaagattgtgcacattgatattgatcctgctgagattggcaagaacaagcagccacatgtgtccatctgtgcagatgttaagcttgctttgcagggcatgaatactcttctggaaggaagcacatcaaagaagagctttgacttcggctcatggcatgatgaattggatcagcaaaagagggagtttccccttgggtataaaatcttcaatgaggaaatccagccacaatatgctattcaggttcttgatgagttgacgaaggggaaggccatcattgccacaggtgttgggcagcaccagatgtgggcggcacagtattacacttacaagcggccaaggcagtggctgtcttcagctggtcttggggctatgggatttggtttgccggctgctgctggtgctgctgtggccaacccaggtgtcactgttgttgacatcgacggagatggtagcttcctcatgaacattcaggagctagctatgatccgtattgagaacctcccagtcaaggtctttgtgctaaacaaccagcacctcgggatggtggtgcagtgggaggacaggttctataaggccaatagagcacacacattcttgggaaacccagagaacgaaagtgagatatatccagattttgtggcaattgccaaagggttcaacattccagcagtccgtgtgacaaagaagagcgaagtccatgcagcaatcaagaagatgcttgaggctccagggccgtacctcttggatataatcgtcccgcaccaggagcatgtgttgcctatgatccctagtggtggggctttcaaggatatgatcctggatggtgatggcaggactgtgtattga

SEQ ID NO: 14 - DNA, 1917 bp, Zea mays (var. A188)
atggccaccgccgccgccgcgtctaccgcgctcactggcgccactaccgctgcgcccaaggcgaggcgccgggcgcacctcctggccacccgccgcgccctcgccgcgcccatcaggtgctcagcggcgtcacccgccatgccgatggctcccccggccaccccgctccggccgtggggccccaccgagccccgcaagggtgctgacatcctcgtcgagtccctcgagcgctgcggcgtccgcgacgtcttcgcctaccccggcggcgcgtccatggagatccaccaggcactcacccgctcccccgtcatcgccaaccacctcttccgccacgagcaaggggaggcctttgccgcctccggctacgcgcgctcctcgggccgcgtcggcgtctgcatcgccacctccggccccggcgccaccaacctagtctccgcgctcgccgacgcgctgctcgattccgtccccatggtcgccatcacgggacaggtgccgcgacgcatgattggcaccgacgccttccaggagacgcccatcgtcgaggtcacccgctccatcaccaagcacaactacctggtcctcgacgtcgacgacatcccccgcgtcgtgcaggaggctttcttcctcgcctcctctggtcgaccagggccggtgcttgtcgacatccccaaggacatccagcagcagatggcggtgcctgtctgggacaagcccatgagtctgcctgggtacattgcgcgccttcccaagccccctgcgactgagttgcttgagcaggtgctgcgtcttgttggtgaatcgcggcgccctgttctttatgtgggcggtggctgcgcagcatctggtgaggagttgcgacgctttgtggagctgactggaatcccggtcacaactactcttatgggcctcggcaacttccccagcgacgacccactgtctctgcgcatgctaggtatgcatgggacggtgtatgcaaattatgcagtggataaggccgatctgttgcttgcacttggtgtgcggtttgatgatcgcgtgacagggaagattgaggcttttgcaagcagggctaagattgtgcacgttgatattgatccggctgagattggcaagaacaagcagccacatgtgtccatctgtgcagatgttaagcttgctttgcagggcatgaatgctcttcttgaaggaagcacatcaaagaagagctttgactttggctcatggaacgatgagttggatcagcagaagagggaattcccccttgggtataaaacatctaatgaggagatccagccacaatatgctattcaggttcttgatgagctgacgaaaggcgaggccatcatcggcacaggtgttgggcagcaccagatgtgggcggcacagtactacacttacaagcggccaaggcagtggttgtcttcagctggtcttggggctatgggatttggtttgccggctgctgctggtgcttctgtggcaaacccaggtgtcactgttgttgacatcgatggagatggtagctttctcatgaacgttcaggagctagctatgatccgaattgagaacctcccagtgaaggtctttgtgctaaacaaccagcacctggggatggtggtgcagtgggaggacaggttctataaggccaacagagcgcacacatacttgggaaacccagagaatgaaagtgagatatatccagatttcgtgacgatcgccaaagggttcaacattccagcggtccgtgtgacaaagaagaacgaagtccgcgcagcgataaagaagatgctcgagactccagggccgtacctcttggatataatcgtcccacaccaggagcatgtgttgcctatgatccctagtggtggggctttcaaggatatgatcctggatggtgatggcaggactgtgtactga

SEQ ID NO: 15 - DNA, 572 bp, Zea mays
ccttcttttgtttcacttatcagttttcaccaagattgcatgccacttggcgggttcatcacgaagaaccttccaataatgcaaaactgtggacggaccttttttttagaataatcgttctaacaaaattgcatagccctgtctgtctcttagttgatcatcagattggccagtagcataaactgaggtagccttggtccatgcggcaaaaaaaaaagaaccatctctctctcttttgccagccaggtacactattgtgttttacctcttttttagtctaaaaaatacttgtcatttattttctatccagctgacaatctttatggcccctgacgaaatagttcgtggcaaactggcaatggtcttttgcaaaacacgacgctgtgcacgtcagcacgtgagcttgcgatgtccactagggagctccatccactgatccacccccacgcggcgtggcgtcgtcattaacggcttgtggggaagggaacgagcaactaaccgataattagtaccagaccggccagtgaacgatgccaaaaccggcttataagctcagctgcgacaaccgtttt

SEQ ID NO: 16 - DNA, 704 bp, Artificial
  ZmU6_pegRNA_488forw
ccttcttttgtttcacttatcagttttcaccaagattgcatgccacttggcgggttcatcacgaagaaccttccaataatgcaaaactgtggacggaccttttttttagaataatcgttctaacaaaattgcatagccctgtctgtctcttagttgatcatcagattggccagtagcataaactgaggtagccttggtccatgcggcaaaaaaaaaagaaccatctctctctcttttgccagccaggtacactattgtgttttacctcttttttagtctaaaaaatacttgtcatttattttctatccagctgacaatctttatggcccctgacgaaatagttcgtggcaaactggcaatggtcttttgcaaaacacgacgctgtgcacgtcagcacgtgagcttgcgatgtccactagggagctccatccactgatccacccccacgcggcgtggcgtcgtcattaacggcttgtggggaagggaacgagcaactaaccgataattagtaccagaccggccagtgaacgatgccaaaaccggcttataagctcagctgcgacaaccgttttgcatggtcgccatcacgggacgttttagagctagaaatagcaagttaaaataaggctagtccgttatcaacttgaaaaagtggcaccgagtcggtgcgtcgcgacacctgtcccgtgatggcgacttttttt

SEQ ID NO: 17 - DNA, 702 bp, Artificial
  ZmU6_pegRNA_493rev
ccttcttttgtttcacttatcagttttcaccaagattgcatgccacttggcgggttcatcacgaagaaccttccaataatgcaaaactgtggacggaccttttttttagaataatcgttctaacaaaattgcatagccctgtctgtctcttagttgatcatcagattggccagtagcataaactgaggtagccttggtccatgcggcaaaaaaaaaagaaccatctctctctcttttgccagccaggtacactattgtgttttacctcttttttagtctaaaaaatacttgtcatttattttctatccagctgacaatctttatggcccctgacgaaatagttcgtggcaaactggcaatggtcttttgcaaaacacgacgctgtgcacgtcagcacgtgagcttgcgatgtccactagggagctccatccactgatccacccccacgcggcgtggcgtcgtcattaacggcttgtggggaagggaacgagcaactaaccgataattagtaccagaccggccagtgaacgatgccaaaaccggcttataagctcagctgcgacaaccgttttgtcggtgccaatcatgcgtcggttttagagctagaaatagcaagttaaaataaggctagtccgttatcaacttgaaaaagtggcaccgagtcggtgcacaggtgtcgcgacgcatgattggcattttttt

SEQ ID NO: 18 - DNA, 702 bp, Artificial
  ZmU6_pegRNA_1858rev
ccttcttttgtttcacttatcagttttcaccaagattgcatgccacttggcgggttcatcacgaagaaccttccaataatgcaaaactgtggacggaccttttttttagaataatcgttctaacaaaattgcatagccctgtctgtctcttagttgatcatcagattggccagtagcataaactgaggtagccttggtccatgcggcaaaaaaaaaagaaccatctctctctcttttgccagccaggtacactattgtgttttacctcttttttagtctaaaaaatacttgtcatttattttctatccagctgacaatctttatggcccctgacgaaatagttcgtggcaaactggcaatggtcttttgcaaaacacgacgctgtgcacgtcagcacgtgagcttgcgatgtccactagggagctccatccactgatccacccccacgcggcgtggcgtcgtcattaacggcttgtggggaagggaacgagcaactaaccgataattagtaccagaccggccagtgaacgatgccaaaaccggcttataagctcagctgcgacaaccgttttgtccttgaaagccccaccactgttttagagctagaaatagcaagttaaaataaggctagtccgttatcaacttgaaaaagtggcaccgagtcggtgctatgatccctaatggtggggctttcattttttt

SEQ ID NO: 19 - DNA, 700 bp, Artificial
  ZmU6_pegRNA_1863forw
ccttcttttgtttcacttatcagttttcaccaagattgcatgccacttggcgggttcatcacgaagaaccttccaataatgcaaaactgtggacggaccttttttttagaataatcgttctaacaaaattgcatagccctgtctgtctcttagttgatcatcagattggccagtagcataaactgaggtagccttggtccatgcggcaaaaaaaaaagaaccatctctctctcttttgccagccaggtacactattgtgttttacctcttttttagtctaaaaaatacttgtcatttattttctatccagctgacaatctttatggcccctgacgaaatagttcgtggcaaactggcaatggtcttttgcaaaacacgacgctgtgcacgtcagcacgtgagcttgcgatgtccactagggagctccatccactgatccacccccacgcggcgtggcgtcgtcattaacggcttgtggggaagggaacgagcaactaaccgataattagtaccagaccggccagtgaacgatgccaaaaccggcttataagctcagctgcgacaaccgttttgtgttgcctatgatccctaggttttagagctagaaatagcaagttaaaataaggctagtccgttatcaacttgaaaaagtggcaccgagtcggtgcgccccaccattagggatcataggcattttttt

SEQ ID NO: 20 - DNA, 675 bp, Artificial
  ZmU6_gRNA_443rev
ccttcttttgtttcacttatcagttttcaccaagattgcatgccacttggcgggttcatcacgaagaaccttccaataatgcaaaactgtggacggaccttttttttagaataatcgttctaacaaaattgcatagccctgtctgtctcttagttgatcatcagattggccagtagcataaactgaggtagccttggtccatgcggcaaaaaaaaaagaaccatctctctctcttttgccagccaggtacactattgtgttttacctcttttttagtctaaaaaatacttgtcatttattttctatccagctgacaatctttatggcccctgacgaaatagttcgtggcaaactggcaatggtcttttgcaaaacacgacgctgtgcacgtcagcacgtgagcttgcgatgtccactagggagctccatccactgatccacccccacgcggcgtggcgtcgtcattaacggcttgtggggaagggaacgagcaactaaccgataattagtaccagaccggccagtgaacgatgccaaaaccggcttataagctcagctgcgacaaccgttttgacggaatcgagcagcgcgtgttttagagctagaaatagcaagttaaaataaggctagtccgttatcaacttgaaaaagtggcaccgagtcggtgcttttttt

SEQ ID NO: 21 - DNA, 676 bp, Artificial
  ZmU6_gRNA_542forw
ccttcttttgtttcacttatcagttttcaccaagattgcatgccacttggcgggttcatcacgaagaaccttccaataatgcaaaactgtggacggaccttttttttagaataatcgttctaacaaaattgcatagccctgtctgtctcttagttgatcatcagattggccagtagcataaactgaggtagccttggtccatgcggcaaaaaaaaaagaaccatctctctctcttttgccagccaggtacactattgtgttttacctcttttttagtctaaaaaatacttgtcatttattttctatccagctgacaatctttatggcccctgacgaaatagttcgtggcaaactggcaatggtcttttgcaaaacacgacgctgtgcacgtcagcacgtgagcttgcgatgtccactagggagctccatccactgatccacccccacgcggcgtggcgtcgtcattaacggcttgtggggaagggaacgagcaactaaccgataattagtaccagaccggccagtgaacgatgccaaaaccggcttataagctcagctgcgacaaccgttttgccaggagacgcccatcgtcggttttagagctagaaatagcaagttaaaataaggctagtccgttatcaacttgaaaaagtggcaccgagtcggtgcttttttt

SEQ ID NO: 22 - DNA, 676 bp, Artificial
  ZmU6_gRNA_1800forw
ccttcttttgtttcacttatcagttttcaccaagattgcatgccacttggcgggttcatcacgaagaaccttccaataatgcaaaactgtggacggaccttttttttagaataatcgttctaacaaaattgcatagccctgtctgtctcttagttgatcatcagattggccagtagcataaactgaggtagccttggtccatgcggcaaaaaaaaaagaaccatctctctctcttttgccagccaggtacactattgtgttttacctcttttttagtctaaaaaatacttgtcatttattttctatccagctgacaatctttatggcccctgacgaaatagttcgtggcaaactggcaatggtcttttgcaaaacacgacgctgtgcacgtcagcacgtgagcttgcgatgtccactagggagctccatccactgatccacccccacgcggcgtggcgtcgtcattaacggcttgtggggaagggaacgagcaactaaccgataattagtaccagaccggccagtgaacgatgccaaaaccggcttataagctcagctgcgacaaccgttttgaagaagatgctcgagactccgttttagagctagaaatagcaagttaaaataaggctagtccgttatcaacttgaaaaagtggcaccgagtcggtgcttttttt

SEQ ID NO: 23 - DNA, 675 bp, Artificial
  ZmU6_gRNA_1809rev
ccttcttttgtttcacttatcagttttcaccaagattgcatgccacttggcgggttcatcacgaagaaccttccaataatgcaaaactgtggacggaccttttttttagaataatcgttctaacaaaattgcatagccctgtctgtctcttagttgatcatcagattggccagtagcataaactgaggtagccttggtccatgcggcaaaaaaaaaagaaccatctctctctcttttgccagccaggtacactattgtgttttacctcttttttagtctaaaaaatacttgtcatttattttctatccagctgacaatctttatggcccctgacgaaatagttcgtggcaaactggcaatggtcttttgcaaaacacgacgctgtgcacgtcagcacgtgagcttgcgatgtccactagggagctccatccactgatccacccccacgcggcgtggcgtcgtcattaacggcttgtggggaagggaacgagcaactaaccgataattagtaccagaccggccagtgaacgatgccaaaaccggcttataagctcagctgcgacaaccgttttgtgggacgattatatccaaggttttagagctagaaatagcaagttaaaataaggctagtccgttatcaacttgaaaaagtggcaccgagtcggtgcttttttt

SEQ ID NO: 24 - DNA, 1992 bp, Zea mays
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 25 - DNA, 250 bp, Agrobacterium tumefaciens
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactag

SEQ ID NO: 26 - DNA, 22 bp, Artificial
  ZmALS_165_for
ccgtcatcgccaaccacctctt

SEQ ID NO: 27 - DNA, 20 bp, Artificial
  ZmALS_165_rev
ttggtgatggagcgggtgac

SEQ ID NO: 28 - DNA, 25 bp, Artificial
  ZmALS_621_for
gaaagtgagatatatccagatttcg

SEQ ID NO: 29 - DNA, 24 bp, Artificial
  ZmALS_621_rev
tcagtacacagtcctgccatcacc

SEQ ID NO: 30 - DNA, 2145 bp, Triticum aestivum (var. Fielder)
gcatttgaaactgcagtgcagaagtcatggtctaacatttctagtgacaataaccgatgttatgttaaagcaacggagctggtgtttgctcaaaagaatgggtcatggggcactcctgtaattcctatggagcgtcctgctgggctcaatgacattggtatggtagcttggatcttggacatgtccactcctgaatatcccaatggcaggcagattgttgtcatcgcaaatgatattacttttagagctggatcgtttggtccaagggaagatgcattttttgaaactgttaccaacctagcttgtgagaggaagcttcctctcatctacttggcagcaaactctggtgctcggatcggcatagcagatgaagtaaaatcgtgcttccgtgttggatggtctgatgatggcagccctgaacgtgggtttcagtacatttatctgactgaagaagaccatgctcgtattagcacttctgttatagcgcacaagatgcagcttgataatggtgaaattaggtgggttattgattctgttgtggggaaggaggatgggctaggtgtggagaacatacatggaagtgctgctattgccagtgcctattctagggcctatgaggagacatttacgcttacatttgtgactggacggactgttggaataggagcatatcttgctcgacttggcatacggtgcatacagcgtactgaccagcccattatcctaaccgggttctctgctttgaacaagcttcttggccgggaagtgtacagctcccacatgcagttgggtggccccaaaattatggcgacaaacggtgttgtccatctgacagtttcagatgaccttgaaggtgtgtctaatatattgaggtggctcagctatgttcctgccaacattggtggacctcttcctattacaaaatctttggacccacctgacagacccgttgcatatatccctgagaatacatgtgatcctcgtgcagccatcagtggcattgatgatagccaagggaaatggttggggggcatgttcgacaaagacagttttgtggagacatttgaaggatgggcgaagtcagtagttactggcagagcgaaactcggagggattccggtgggtgttatagctgtggagacacagactatgatgcagctcatccctgctgatccaggccagcttgattcccatgagcggtctgttcctcgtgctgggcaagtctggtttccagattcagttactaagacagctcaagcaatgctggacttcaaccgtgaaggattacctctgttcatccttgctaactggagaggcttctctggtgggcaaagagatctttttgaaggaatccttcaggctgggtcaacaattgttgagaaccttaggacatacaatcagcctgcctttgtatatatccccaaggctgcagagctacgtggaggggcttgggtcgtgattgatagcaagataaatccagatcgaattgagttctatgctgagaggactgcaaagggtaatgttcttgaacctcaagggttgattgagatcaagttcaggtcagaggaactccaagagtgcatgggcaggcttgacccagaattgataaatctgaaggcaaaactcctgggagcaaagcatgaaaatggaagtctatctgagtcagaatcccttcagaagagcatagaagcccggaagaaacagttgttgcctttgtatactcaaattgcggtgcggtttgctgaattgcatgacacttcccttagaatggctgctaagggtgtgattaagaaggttgtagactgggaagattctaggtctttcttctacaagagattacggaggaggatatccgaggacgttcttgcaaaggaaattagaggtgtaagtggcaagcagttctctcaccaatcagcaatcgagctgatccagaaatggtacttggcttctaagggagctgaagcagcaagcactgaatgggatgatgacgatgcttttgttgcctggagggaaaaccctgaaaactaccaggagtatatcaaagaacttagggctcaaagggtatctcagttgctctcagatgttgcagactccagtccagatctagaagccttgccacagggtctttctatgctactagagaag

SEQ ID NO: 31 - DNA, 2145 bp, Triticum aestivum (var. Fielder)
gcatttgaaactgcagtgcagaagtcatggtctaacatttctagtgacaataaccgatgttatgttaaagcaacggagctggtgtttgctcacaagaatgggtcatggggcactcctgtaattcctatggagcgtcctgctgggctcaatgacattggtatggtagcttggatcttggacatgtccactcctgaatatcccaatggcaggcagattgttgtcatcgcaaatgatattacttttagagctggatcgtttggtccaagggaagatgcattttttgaaactgttaccaacctagcttgtgagaggaagcttcctctcatctacttggcagcaaactctggtgctcggatcggcatagcagatgaagtaaaatcttgcttccgtgttggatggtctgatgatggcagccctgaacgtgggtttcagtacatttatctgactgaagaagaccatgctcgtattagcacttctgttatagcgcacaagatgcagcttgataatggtgaaattaggtgggttatcgattctgttgtggggaaggaggatgggctaggtgtggagaacatacatggaagtgctgctattgccagtgcctattctagggcctatgaggagacatttacgcttacatttgtgactggacggactgttggaataggagcatatcttgctcgacttggcatacggtgcatacagcgtactgaccagcccattatcctaactgggttctctgccttgaacaagcttcttggccgggaagtttacagctcccacatgcagttgggtggccccaaaattatggcgacaaacggtgttgtccatctgacagtttcagatgaccttgaaggtgtatctaatatattgaggtggctcagctatgttcctgccaacattggtggacctcttcctattacaaaatctttggacccacctgacagacccgttgcttacatccctgagaatacatgcgatcctcgtgctgccatcagtggcattgatgatagccaagggaaatggttggggggcatgttcgacaaagacagttttgtggagacatttgaaggatgggcgaagtcagttgttactggcagagctaaactcggagggattccggtgggtgttatagctgtggagacacagactatgatgcagctcatccctgctgatccaggccagcttgattcccatgagcgatctgttcctcgtgctgggcaagtctggtttccagattcagctactaagacagcgcaggcaatgctggacttcaaccgtgaaggattacctctgttcatccttgctaactggagaggcttctctggtggacaaagagatctttttgaaggaatccttcaggctgggtcaacaattgttgagaaccttaggacatacaatcagcctgcctttgtatatatccccaaggctgcagagctacgtggaggggcttgggtcgtgattgatagcaagataaatccagatcgcattgagttctatgctgagaggactgcaaagggcaatgttctcgaacctcaagggttgatcgagatcaagttcaggtcagaggaactccaagagtgcatgggtaggcttgatccagaattgataaatctgaaggcaaagctccagggagtaaagcatgaaaatggaagtctacctgagtcagaatcccttcagaagagcatagaagcccggaagaaacagttgttgcctttgtatactcaaattgcggtacggttcgctgaattgcatgacacttcccttagaatggctgctaagggtgtgattaagaaggttgtagactgggaagattctaggtcgttcttctacaagagattacggaggaggatatccgaggatgttcttgcgaaggaaattagaggtgtaagtggcaagcagttttctcaccaatcggcaatcgagctgatccagaaatggtacttggcctctaagggagctgaaacaggaagcactgaatgggatgatgacgatgcttttgttgcctggagggaaaaccctgaaaactaccaggagtatatcaaagaactcagggctcaaagggtatctcagttgctctcagatgttgcagactccagtccagatctagaagccttgccacagggtctttctatgctactagagaag

SEQ ID NO: 32 - DNA, 2145 bp, Triticum aestivum (var. Fielder)
gcatttgaaactgcagtgcagaagtcatggtctaacatttctagtgacactaaccgatgttatgttaaagcgacggagctggtgtttgctcacaagaacgggtcatggggcactcctgtaattcctatggagcgtcctgctgggctcaatgacattggtatggtagcttggatcttggacatgtccactcctgaatatcccaatggcaggcagattgttgtcatcgcaaatgatattacttttagagctggatcgtttggtccaagggaagatgcattttttgaaactgttaccaacctagcttgtgagaggaagcttcctctcatctacttggcagcaaactctggtgctcggatcggcatagcagatgaagtaaaatcttgcttccgtgttggatggtctgatgatggcagccctgaacgtgggtttcaatatatttatctgactgaagaagaccatgctcgtattagcgcttctgttatagcgcacaagatgcagcttgataatggtgaaattaggtgggttattgattctgttgtagggaaggaggatgggctaggtgtggagaacatacatggaagtgctgctattgccagtgcctattctagggcctatgaggagacatttacgcttacatttgtgactggaaggactgttggaataggagcatatcttgctcgacttggcatacggtgcattcagcgtactgaccagcccattatcctaactgggttttctgccttgaacaagcttcttggccgggaagtgtacagctcccacatgcagttgggtggccccaaaattatggccacaaacggtgttgtccatctgacagtttcagatgaccttgaaggtgtatctaatatattgaggtggctcagctatgttcctgccaacattggtggacctcttcctattacaaaatctttggacccacctgacagacccgttgcttacatccctgagaatacatgtgatcctcgtgcagccatcagtggcattgatgatagccaagggaaatggttggggggcatgttcgacaaagacagttttgtggagacatttgaaggatgggcgaagtcagtagttactggcagagcgaaactcggagggattccggtgggtgttatagctgtggagacacagactatgatgcagctcatccctgctgatccaggtcagcttgattcccatgagcggtctgttcctcgtgctgggcaagtctggtttccagattcagctactaagacagcgcaggcaatgctggacttcaaccgtgaaggattacctctgttcatccttgctaactggagaggcttctctggtgggcaaagagatctttttgaaggaatccttcaggctgggtcaacaattgttgagaaccttaggacatacaatcagcctgcctttgtatatatccccaaggctgcagagctacgtggaggggcttgggtcgtgattgatagcaagataaatccagatcgcattgagttctatgctgagaggactgcaaagggcaatgttcttgaacctcaagggttgattgagatcaagttcaggtcagaggaactccaagagtgcatgggcaggcttgacccagaattgataaatttgaaggcaaaactcctgggagcaaagcatgaaaatggaagtctatctgagtcagaatcccttcagaagagcatagaagcccggaagaaacagttgttgcctttgtatactcaaattgcggtacggttcgctgaattgcatgacacttcccttagaatggctgctaagggtgtgattaagaaggttgtagactgggaagattctaggtctttcttctacaagagattacggaggaggatatccgaggatgttcttgcaaaggaaattagaggtgtaagtggcaagcagttttctcaccaatcggcaatcgagctgatccagaaatggtacttggcctctaagggagctgaaacaggaaacactgaatgggatgatgacgatgcttttgttgcctggagggaaaaccctgaaaactaccaggagtatatcaaagaactcagggctcaaagggtatctcagttgctctcagatgttgcagactccagtccagatctagaagccttgccacagggtctttctatgctactagagaag

SEQ ID NO: 33 - DNA, 362 bp, Triticum aestivum
gaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagactt

SEQ ID NO: 34 - DNA, 527 bp, Artificial
  TaU6_pegRNA_1275rev
gaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttgtgaacagaggtaatccttcagttttagagctagaaatagcaagttaaaataaggctagtccgttatcaacttgaaaaagtggcaccgagtcggtgcagattcagttactaagacagctcaagcaatgctggacttcaaccgtgaaggattacctctgttttttt

SEQ ID NO: 35 - DNA, 465 bp, Artificial
  TaU6_gRNA_1178forw
gaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttgccagcttgattcccatgaggttttagagctagaaatagcaagttaaaataaggctagtccgttatcaacttgaaaaagtggcaccgagtcggtgcttttttt

SEQ ID NO: 36 - DNA, 466 bp, Artificial
  TaU6_pegRNA_1205forw
gaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttgttcctcgtgctgggcaagtcgttttagagctagaaatagcaagttaaaataaggctagtccgttatcaacttgaaaaagtggcaccgagtcggtgcttttttt

SEQ ID NO: 37 - DNA, 19 bp, Artificial
  TaACCase_forw
atgatgcagctcatccctg

SEQ ID NO: 38 - DNA, 19 bp, Artificial
  TaACCase_rev
ttgttgacccagcctgaag

SEQ ID NO: 39 - protein, 1368 aa, Artificial
  nCas9(H840A)
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 40 - protein, 2117 aa, Artificial
  PE2 editor
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 41 - DNA, 20 bp, Artificial
  Spacer for pegRNA used to modify ZmALS2
catggtcgccatcacgggac

SEQ ID NO: 42 - DNA, 15 bp, Artificial
  RT-template of pegRNA used to modify ZmALS2
gtcgcgacacctgtc

SEQ ID NO: 43 - DNA, 13 bp, Artificial
  Primer Binding Site of pegRNA used to modify ZmALS2
ccgtgatggcgac

SEQ ID NO: 44 - DNA, 20 bp, Artificial
  Spacer for pegRNA used to modify ZmALS2
tcggtgccaatcatgcgtcg

SEQ ID NO: 45 - DNA, 13 bp, Artificial
  RT-template of pegRNA used to modify ZmALS2
acaggtgtcgcga

SEQ ID NO: 46 - DNA, 13 bp, Artificial
  Primer Binding Site of pegRNA used to modify ZmALS2
cgcatgattggca

SEQ ID NO: 47 - DNA, 20 bp, Artificial
  Spacer for pegRNA used to modify ZmALS2
tccttgaaagccccaccact

SEQ ID NO: 48 - DNA, 13 bp, Artificial
  RT-template of pegRNA used to modify ZmALS2
tatgatccctaat

SEQ ID NO: 49 - DNA, 13 bp, Artificial
  Primer Binding Site of pegRNA used to modify ZmALS2
ggtggggctttca

SEQ ID NO: 50 - DNA, 20 bp, Artificial
  Spacer for pegRNA used to modify ZmALS2
gtgttgcctatgatccctag

SEQ ID NO: 51 - DNA, 12 bp, Artificial
  RT-template of pegRNA used to modify ZmALS2
gccccaccatta

SEQ ID NO: 52 - DNA, 13 bp, Artificial
  Primer Binding Site of pegRNA used to modify ZmALS2
gggatcataggca

SEQ ID NO: 53 - DNA, 20 bp, Artificial
  Spacer for gRNA used to modify ZmALS2
gacggaatcgagcagcgcgt

SEQ ID NO: 54 - DNA, 20 bp, Artificial
  Spacer for gRNA used to modify ZmALS2
ccaggagacgcccatcgtcg

SEQ ID NO: 55 - DNA, 20 bp, Artificial
  Spacer for gRNA used to modify ZmALS2
aagaagatgctcgagactcc

SEQ ID NO: 56 - DNA, 20 bp, Artificial
  Spacer for gRNA used to modify ZmALS2
gtgggacgattatatccaag

SEQ ID NO: 57 - DNA, 20 bp, Artificial
  Spacer for pegRNA used to modify TaACCase
tgaacagaggtaatccttca

SEQ ID NO: 58 - DNA, 48 bp, Artificial
  RT-template of pegRNA used to modify TaACCase
agattcagttactaagacagctcaagcaatgctggacttcaaccgtga

SEQ ID NO: 59 - DNA, 13 bp, Artificial
  Primer Binding Site of pegRNA used to modify TaACCase
aggattacctctg

SEQ ID NO: 60 - DNA, 20 bp, Artificial
  Spacer for gRNA used to modify TaACCase
gccagcttgattcccatgag

SEQ ID NO: 61 - DNA, 20 bp, Artificial
  Spacer for gRNA used to modify TaACCase
ttcctcgtgctgggcaagtc

SEQ ID NO: 62 - DNA, 2404 bp, Physcomitrella patens
gccgtggcgcatcttgtggacttgccgtggcgagctacctccacgctcccgcttccgcaattttgttgcagtgctttggattctcgtggttgctcgtctcaggtggttcacttcttaacttgcttcgttacgcgccgccttctcctgcattgtctcttcatatatatgtatatatttttttttcttttcacttttgttgagctcggtgagggtatctagttttggtgaataaatgtgcagagtgtagaatttgctgtgggttatgggcacagtgcctgtgatgcggttgttgttgttgttgttggagaagccctttgttttgggctcgagacttgaataaaatatgggacggtactggttccattttgtagaattatgattatgatgatggtcaatgtggcagcaagtaggaggattggatttgaattgtgggtttttgcgctcgctgtttctgagttagttactgacgaagctctgtaaattgtgtgtggacaggaagtgaagatgtcggcctccaaggatggcgaccctcggatccagtatatctcggacagcatccgtaccattcctgattttcctcacaaaggtactatgctccacgccatagtcgtcactctcggttttgttttgcagtgttcgtgacatgtggtttttttcttttttcacctttagcgaaggttttgtatggaattctctttcgtagcgcgagtttacgtgatgaatttggtgcaggcattatgttccgagatgtgacgacgttgctgttggatcataaggctttcaaagacacgatcgacatctttgttgagcgttaccgggaccagaaggtggacgtcattgtgggtgcgatgcccttgactcccttgacgtaccataacggttttgtgtaataatttgaaggcaaccgttacgtttataaatttgggaagattgtctggctgttgttgattactttcgtgctacttttttcaggaattgaagctcgaggttttatctttgggccacccattgctcttgccatcggtgcgaagtttgtgccaatgcgcaagcctcgtaaacttccaggtaagggttgacaggcactttttctcttaatctcgtgattcatgaaagattagggctctcaatggctttcgtcgtccgtggaatagagttttcaatgtgaccgagacttcatcctcatgtagcttaaaggtgtattcgctttctatttctcaagctgcgaaagcatgtcagcacttattatctagagaagctaaatacattatttcaacatttttgtgatatcgattgctttgtatgtgctttgcatactaagaatcaaaatccttgacaattttgtgctgacgtccgtgttcacatgtcttaggccctgtgattagggaagagtatagtctagagtatggtaccgattgcattgagatgcacgttggagccatcgagaagaacgagcgcgtagttattgttgatgacttaattgccaccggaggcactcttggagctgccatcaaattgctaggtttgctttctttcttcctctcttacttaaatgagaactgttttcgaacatttaagcaggaaactttgtcttcaacaacctgtgaagcatggcgttttgtttagacgttggatccaactacaggaagaaacctaaatgtgtgttttcatgttagcactagttttgtcgaatctttgagatgtttgaccttgaaagttgacaccaccttgtttaacagacaaagttgaagcggagattgtggaatgcgcgtgcttggttgagctggtagacctgaaggtatttttctcccactgcataatctgtagggttgctttctctgaggcaattgtaagagttcaatgccgtctggagcttacagttttatcgtttatcctatgatgacgtagaataaccgaaaatcgtaattatctccagcaagactcctctttttgtgtgttgttaacacatcttcattcttagctacaaagagtttaatgccgtctagtatctgctatgttgatgaacttggaacatatttgcagggtcgtgagaagttgtcgggcaagccgctgtacatcttggtggaatatgagggcgagtaagcttcaccatccccgaactgtcgccgtggtcattggttctatgaagtgccaatcctatatatatggaaacacgatctgcaaacattatggcaaaagcagatcgtactgcaattaattttgagctactatcgattctttgattttcggttttccaaggtgtatgactttgggtcagaattatttagacaacctggttgtctgaacattcatatatacttatctcagaggattatgctttactgggaccttttcttctaacctcggaaacgtaattgtagcatcacctactcctgcattgcaacag

SEQ ID NO: 63 - protein, 184 aa, Physcomitrella patens
MSASKDGDPRIQYISDSIRTIPDFPHKGIMFRDVTTLLLDHKAFKDTIDIFVERYRDQKVDVIVGIEARGFIFGPPIALAIGAKFVPMRKPRKLPGPVIREEYSLEYGTDCIEMHVGAIEKNERVVIVDDLIATGGTLGAAIKLLDKVEAEIVECACLVELVDLKGREKLSGKPLYILVEYEGE

SEQ ID NO: 64 - protein, 968 aa, Oryza sativa
PLLLTASTSIPKSKTFRFENHLLLDRDFLPSVMHRWPQQLNMNDPAACLAAKLKQTRSIIKVWMKEHRKRSSLNEDCKFTIDLLDYIEELRPLNDGERCLRNLVQEKLFQAIQSKVAYWKQRGKVKRLKIGIDNSAFFKAHASKNYRNSRIRSIKHNGAEIADHNGKALLLHSFFQHFLGSDCQPLWDFDLSTLYSEDERDYNLLASQGLQFSTEEIITSIKGMNSNSAPGPDGFGPSFYNSAWNSIKEDIIKLAASFCSNSVQLERINRAHIVLIPKPGKENTVDGYRPISLQNCSVKILSKVLANRLQKVLTRMIHLDQTGFLKGRCISENLIYATELIQACHARKCKTLIIKLDFAKAFDSVLWTSLFQILAVRGFPNNWISWIKSLLQTSKSAVLLNGIPGKWINCKKGLRQGDPLSPYLFILVADVLQRLLEKNFQIRHPIYQDRPCATIQYADDTLVICRAEEDDVLALRSTLLQFSKATGLQINFAKSTMISLHIDRSKESSLSELLQCKLESLPMSYLGLPLSLHKLTNNDLQPIVVKVDSFLTGWEASLLSQAERLILVNAVLSSVPVYAMSAFKLPPKVIEAIDKRRRAFFWTGDDTCSGAKCLVAWDEVCTAKEKGGLGIKSLKTQNEALLLKRLFNLFSDNSSWTNWIWKEFDGRSLLKSLPLGQHWSVFQKLLPELFKITTVHIGDGSRTSFWHDRWTGNMTLAAQFESLFSHSTDDLATVEQILSFGIYDILTPRLSSAAQNDLQELQTILQNFSLTNESDTRLNNRLGNLTTKTIYDLRSPPGFPSPNWKFIWDCRMPLKIKLFAWLLVRDRLSTKLNLLKKKIVQTATCDICSTTDETADHLSFNCPFAISFWQALHIQPIINETKYLSQLKAPATIPTRHFQSFFMLCFWVLWNHRHDVVFRGRPPSIASCLQRGISESSLWAETFTPDDRFVIDVWKGVFSSSLQTLHPM

SEQ ID NO: 65 - protein, 2407 aa, Artificial Sequence
  nCas9-KarmaRT
MAPKKKRKVGIHGVPAAMDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSPLLLTASTSIPKSKTFRFENHLLLDRDFLPSVMHRWPQQLNMNDPAACLAAKLKQTRSIIKVWMKEHRKRSSLNEDCKFTIDLLDYIEELRPLNDGERCLRNLVQEKLFQAIQSKVAYWKQRGKVKRLKIGIDNSAFFKAHASKNYRNSRIRSIKHNGAEIADHNGKALLLHSFFQHFLGSDCQPLWDFDLSTLYSEDERDYNLLASQGLQFSTEEIITSIKGMNSNSAPGPDGFGPSFYNSAWNSIKEDIIKLAASFCSNSVQLERINRAHIVLIPKPGKENTVDGYRPISLQNCSVKILSKVLANRLQKVLTRMIHLDQTGFLKGRCISENLIYATELIQACHARKCKTLIIKLDFAKAFDSVLWTSLFQILAVRGFPNNWISWIKSLLQTSKSAVLLNGIPGKWINCKKGLRQGDPLSPYLFILVADVLQRLLEKNFQIRHPIYQDRPCATIQYADDTLVICRAEEDDVLALRSTLLQFSKATGLQINFAKSTMISLHIDRSKESSLSELLQCKLESLPMSYLGLPLSLHKLTNNDLQPIVVKVDSFLTGWEASLLSQAERLILVNAVLSSVPVYAMSAFKLPPKVIEAIDKRRRAFFWTGDDTCSGAKCLVAWDEVCTAKEKGGLGIKSLKTQNEALLLKRLFNLFSDNSSWTNWIWKEFDGRSLLKSLPLGQHWSVFQKLLPELFKITTVHIGDGSRTSFWHDRWTGNMTLAAQFESLFSHSTDDLATVEQILSFGIYDILTPRLSSAAQNDLQELQTILQNFSLTNESDTRLNNRLGNLTTKTIYDLRSPPGFPSPNWKFIWDCRMPLKIKLFAWLLVRDRLSTKLNLLKKKIVQTATCDICSTTDETADHLSFNCPFAISFWQALHIQPIINETKYLSQLKAPATIPTRHFQSFFMLCFWVLWNHRHDVVFRGRPPSIASCLQRGISESSLWAETFTPDDRFVIDVWKGVFSSSLQTLHPMSGGSKRTADGSEFEPKKKRKV

SEQ ID NO: 66 - DNA, 300 bp, Zea mays
gcatccaatccaatatggacgattggacgtggaaggtttcctgtgtcttctacgatataataattggtcgctgtcctgtgtggatgtttcctgtgtcttctacgatatattaatgtacttctgtctgtttcgtttcgtatgtacaatcttccgggtatcgttcatgtatccctttgaataataacaaataaaatcgggtttgtcacggtaatctgaataaccttttattagccaaagtttaaccaaaattacgtattgcacgtaaaaattaagctagtttttggagagccggagacagac

SEQ ID NO: 67 - DNA, 6351 bp, Artificial Sequence
  nCas9-MMLV_RTmut_PE2
atggctcctaagaagaagcgcaaggttgggatccacggggttccggcggctatggacaagaagtacagcatcgggctcgacatcgggacgaacagcgttgggtgggcggtcatcacggacgagtacaaggttccctccaagaagttcaaggtgctggggaacaccgaccgccactccatcaagaagaacctcatcggggccctcctgttcgacagcggggagaccgctgaggctacgaggctcaagagaaccgcgaggcgcagatacacgagaaggaagaacaggatctgctacctccaagagatcttctccaacgagatggccaaggttgacgattcattcttccaccgcctggaggagtctttcctcgtggaggaggacaagaagcacgagcggcatcccatcttcgggaacatcgtcgacgaggttgcctaccacgagaagtaccctacgatctaccatctgcggaagaagctcgtggacagcaccgataaggcggacctcagactgatctacctcgccctggcgcacatgatcaagttccgcgggcatttcctgatcgagggggatctcaacccagacaactccgatgttgacaagctgttcatccaactcgtgcagacctacaaccaactcttcgaggagaacccgatcaacgcttcgggggtcgacgctaaggccatcctgtcagcccgcctctcgaagtcacgcaggctggagaacctgatcgcgcagctcccaggggagaagaagaacgggctgttcgggaacctcatcgctctctctctggggctcacgccgaacttcaagtccaacttcgatctcgccgaggacgcgaagctgcaactcagcaaggacacctacgacgatgacctcgataacctcctggcgcagatcggggatcaatacgctgacctgttcctcgccgcgaagaacctgtcggacgccatcctcctgtcagatatcctccgcgtgaacaccgagatcacgaaggccccactctctgcgtccatgatcaagcgctacgacgagcaccatcaggatctgaccctcctgaaggcgctggtccgccaacagctcccggagaagtacaaggagatcttcttcgatcagtcgaagaacgggtacgctgggtacatcgacgggggggcgtcacaagaggagttctacaagttcatcaagccaatcctggagaagatggacgggacggaggagctcctggtgaagctcaacagggaggacctcctgcggaagcagagaaccttcgataacgggtccatcccccaccaaatccatctcggggagctgcacgccatcctgagaaggcaagaggacttctaccctttcctcaaggataaccgggagaagatcgagaagatcctgaccttcagaatcccatactacgtcgggccgctcgctcgggggaacagcagattcgcctggatgacccgcaagtcggaggagaccatcacgccgtggaacttcgaggaggtggtcgacaagggggcgagcgctcagtcgttcatcgagaggatgaccaacttcgacaagaacctgcccaacgagaaggtgctccctaagcactcgctcctgtacgagtacttcaccgtctacaacgagctcacgaaggtgaagtatgtgaccgaggggatgagaaagcctgccttcctgtccggggagcagaagaaggcgatcgtggacctcctgttcaagaccaaccggaaggtcacggttaagcaactcaaggaggactacttcaagaagatcgagtgcttcgattcggtggagatctcaggggtcgaggacaggttcaacgcctccctcgggacctaccacgatctcctgaagatcatcaaggataaggacttcctggacaacgaggagaacgaggacatcctggaggacatcgtgctgaccctcacgctgttcgaggacagggagatgatcgaggagcgcctgaagacgtacgcccatctcttcgatgacaaggtcatgaagcaactcaagagaagaagatacaccgggtgggggaggctgtcccgcaagctcatcaacgggatccgggacaagcagtcagggaagaccatcctcgacttcctgaagtctgatgggttcgccaacaggaacttcatgcaactgatccacgatgactcactcaccttcaaggaggatatccaaaaggctcaggtgagcgggcagggggactcgctgcacgagcatatcgctaacctcgctgggtcccctgcgatcaagaaggggatcctgcagaccgtgaaggttgtggacgagctcgtgaaggtcatggggcggcataagcctgagaacatcgtcatcgagatggccagagagaaccaaaccacgcagaaggggcaaaagaactctagggagcgcatgaagcgcatcgaggaggggatcaaggagctggggtcccaaatcctcaaggagcacccagttgagaacacccaactgcagaacgagaagctctacctgtactacctccagaacgggagggatatgtatgtggaccaagagctggatatcaaccgcctcagcgattacgacgttgatgctatcgtgccccagtctttcctgaaggatgactccatcgacaacaaggtcctcaccaggtcggacaagaaccgcgggaagtcagataacgttccatctgaggaggtcgttaagaagatgaagaactactggaggcagctcctgaacgccaagctgatcacgcaaaggaagttcgacaacctcaccaaggctgagagaggggggctctcagagctggacaaggctgggttcatcaagcggcagctggtcgagaccagacaaatcacgaagcacgttgcgcaaatcctcgactctcggatgaacacgaagtacgatgagaacgacaagctgatcagggaggtgaaggtcatcaccctgaagtctaagctcgtctccgacttccgcaaggacttccagttctacaaggttcgcgagatcaacaactaccaccatgcgcatgacgcttacctcaacgctgtggtcgggaccgccctgatcaagaagtacccgaagctggagagcgagttcgtgtacggggactacaaggtttacgatgtgcgcaagatgatcgccaagtcggagcaagagatcgggaaggccaccgccaagtacttcttctactcaaacatcatgaacttctttaagaccgagatcacgctggccaacggggagatccggaagagacccctcatcgagaccaacggggagacgggggagatcgtgtgggacaaggggagggatttcgcgaccgtccgcaaggttctctccatgccccaggtgaacatcgtcaagaagaccgaggtccaaacgggggggttctcaaaggagtctatcctgcctaagcggaacagcgacaagctcatcgccagaaagaaggactgggatccaaagaagtacggggggttcgacagccctaccgtcgcgtactcggttctggttgtggctaaggttgagaaggggaagtccaagaagctcaagagcgtgaaggagctcctggggatcaccatcatggagcgctccagcttcgagaagaacccaatcgacttcctggaggccaaggggtacaaggaggtgaagaaggacctgatcatcaagctcccgaagtactctctcttcgagctggagaacgggaggaagcgcatgctggcttccgctggggagctccaaaaggggaacgagctcgccctgccatccaagtatgtgaacttcctctacctggcgtcccactacgagaagctcaaggggagcccggaggacaacgagcaaaagcagctgttcgtcgagcagcacaagcattacctcgacgagatcatcgagcaaatctccgagttctccaaacgcgtgatcctcgcggacgctaacctggataaggtcctctccgcttacaacaagcaccgggacaagcccatcagagagcaggccgagaacatcatccatctcttcaccctgacgaacctcggggcccctgctgccttcaagtacttcgacaccacgatcgatcggaagagatacaccagcacgaaggaggtcctggacgcgaccctcatccaccaatctatcacggggctgtacgagacgagaatcgacctgtcacaactggggggggactccggcggctccagcggcggctccagcggcagcgagacgccgggcacgtccgagagcgccacccccgagagcagcggcggctccagcggcggctccagcaccctcaacatcgaggacgagtaccgcctgcacgagacgtccaaggagccggacgtgtccctcggcagcacgtggctctcggacttcccccaggcttgggccgagacgggcggcatgggcctcgccgtgaggcaggcgccgctcatcatcccgctgaaggccacctccacccccgtcagcatcaagcagtacccgatgtcgcaggaggccaggctcggcatcaagccccacatccagaggctcctggaccagggcatcctggtcccctgccagagcccatggaacaccccgctcctgccggtgaagaagccgggcaccaacgactacaggcccgtccaggacctcagggaggtgaacaagcgcgtcgaggacatccaccccacggtgccgaacccgtacaacctcctgtcgggcctgccgcccagccaccagtggtacaccgtcctcgacctgaaggacgccttcttctgcctgcggctccaccccacctcgcagcccctcttcgctttcgagtggcgcgaccccgagatgggcatctcgggccagctcacctggacgaggctgccacagggcttcaagaacagcccgaccctgttcaacgaggcgctccacagggacctggcggacttccgcatccagcacccggacctcatcctcctgcagtatgtggacgacctcctcctggcggccacctcggagctggactgccagcagggcaccagggcgctgctccagacgctcggcaacctgggctacagggcttcggcgaagaaggcccagatctgccagaagcaggtgaagtacctcggctacctgctcaaggagggccagcggtggctgacggaggctaggaaggagacggtcatgggccagcccaccccaaagacgcccaggcagctcagggagttcctgggcaaggccggcttctgccgcctcttcatccccggcttcgcggagatggcggcgccgctctacccactgaccaagccgggcacgctgttcaactggggccccgaccagcagaaggcctaccaggagatcaagcaggccctgctcacggctcccgcgctcggcctgcccgacctcacgaagccgttcgagctgttcgtggacgagaagcagggctacgcgaagggcgtcctcacgcagaagctgggcccctggcgcaggcccgtggcctacctctccaagaagctcgaccccgtggcggccggctggccgccatgcctgaggatggtggcggccatcgccgtcctcaccaaggacgcgggcaagctgaccatgggccagcccctcgtgatcctcgcgccgcacgcggtggaggccctcgtcaagcagccgccagacaggtggctgagcaacgcccgcatgacccactaccaggccctgctcctggacaccgacagggtccagttcggcccggtggtggctctgaaccccgccaccctcctgccgctccccgaggagggcctgcagcacaactgcctcgacatcctcgcggaggcccacggcacccgcccggacctcacggaccagccgctgcccgacgccgaccacacctggtacaccgacggctccagcctcctgcaggagggccagaggaaggcgggcgctgcggtgaccacggagacggaggtcatctgggccaaggcgctcccggcgggcacctccgctcagcgcgcggagctcatcgccctgacgcaggcgctcaagatggccgagggcaagaagctgaacgtgtacacggactcccgctacgccttcgccaccgctcacatccacggcgagatctacaggaggaggggctggctgacctcggagggcaaggagatcaagaacaaggacgagatcctcgcgctcctgaaggccctcttcctgcccaagcgcctgtccatcatccactgccccggccaccagaagggccactcggccgaggccaggggcaacaggatggcggaccaggcggccaggaaggcggccatcacggagacgccggacaccagcacgctcctgatcgagaactccagcccgtccggcggcagcaagcgcacggcggacggctcggagttcgagccgaagaagaagcgcaaggtgtga

SEQ ID NO: 68 - DNA, 5862 bp, Artificial Sequence
  nCas9-CaMVRT
atggctcctaagaagaagcgcaaggttgggatccacggggttccggcggctatggacaagaagtacagcatcgggctcgacatcgggacgaacagcgttgggtgggcggtcatcacggacgagtacaaggttccctccaagaagttcaaggtgctggggaacaccgaccgccactccatcaagaagaacctcatcggggccctcctgttcgacagcggggagaccgctgaggctacgaggctcaagagaaccgcgaggcgcagatacacgagaaggaagaacaggatctgctacctccaagagatcttctccaacgagatggccaaggttgacgattcattcttccaccgcctggaggagtctttcctcgtggaggaggacaagaagcacgagcggcatcccatcttcgggaacatcgtcgacgaggttgcctaccacgagaagtaccctacgatctaccatctgcggaagaagctcgtggacagcaccgataaggcggacctcagactgatctacctcgccctggcgcacatgatcaagttccgcgggcatttcctgatcgagggggatctcaacccagacaactccgatgttgacaagctgttcatccaactcgtgcagacctacaaccaactcttcgaggagaacccgatcaacgcttcgggggtcgacgctaaggccatcctgtcagcccgcctctcgaagtcacgcaggctggagaacctgatcgcgcagctcccaggggagaagaagaacgggctgttcgggaacctcatcgctctctctctggggctcacgccgaacttcaagtccaacttcgatctcgccgaggacgcgaagctgcaactcagcaaggacacctacgacgatgacctcgataacctcctggcgcagatcggggatcaatacgctgacctgttcctcgccgcgaagaacctgtcggacgccatcctcctgtcagatatcctccgcgtgaacaccgagatcacgaaggccccactctctgcgtccatgatcaagcgctacgacgagcaccatcaggatctgaccctcctgaaggcgctggtccgccaacagctcccggagaagtacaaggagatcttcttcgatcagtcgaagaacgggtacgctgggtacatcgacgggggggcgtcacaagaggagttctacaagttcatcaagccaatcctggagaagatggacgggacggaggagctcctggtgaagctcaacagggaggacctcctgcggaagcagagaaccttcgataacgggtccatcccccaccaaatccatctcggggagctgcacgccatcctgagaaggcaagaggacttctaccctttcctcaaggataaccgggagaagatcgagaagatcctgaccttcagaatcccatactacgtcgggccgctcgctcgggggaacagcagattcgcctggatgacccgcaagtcggaggagaccatcacgccgtggaacttcgaggaggtggtcgacaagggggcgagcgctcagtcgttcatcgagaggatgaccaacttcgacaagaacctgcccaacgagaaggtgctccctaagcactcgctcctgtacgagtacttcaccgtctacaacgagctcacgaaggtgaagtatgtgaccgaggggatgagaaagcctgccttcctgtccggggagcagaagaaggcgatcgtggacctcctgttcaagaccaaccggaaggtcacggttaagcaactcaaggaggactacttcaagaagatcgagtgcttcgattcggtggagatctcaggggtcgaggacaggttcaacgcctccctcgggacctaccacgatctcctgaagatcatcaaggataaggacttcctggacaacgaggagaacgaggacatcctggaggacatcgtgctgaccctcacgctgttcgaggacagggagatgatcgaggagcgcctgaagacgtacgcccatctcttcgatgacaaggtcatgaagcaactcaagagaagaagatacaccgggtgggggaggctgtcccgcaagctcatcaacgggatccgggacaagcagtcagggaagaccatcctcgacttcctgaagtctgatgggttcgccaacaggaacttcatgcaactgatccacgatgactcactcaccttcaaggaggatatccaaaaggctcaggtgagcgggcagggggactcgctgcacgagcatatcgctaacctcgctgggtcccctgcgatcaagaaggggatcctgcagaccgtgaaggttgtggacgagctcgtgaaggtcatggggcggcataagcctgagaacatcgtcatcgagatggccagagagaaccaaaccacgcagaaggggcaaaagaactctagggagcgcatgaagcgcatcgaggaggggatcaaggagctggggtcccaaatcctcaaggagcacccagttgagaacacccaactgcagaacgagaagctctacctgtactacctccagaacgggagggatatgtatgtggaccaagagctggatatcaaccgcctcagcgattacgacgttgatgctatcgtgccccagtctttcctgaaggatgactccatcgacaacaaggtcctcaccaggtcggacaagaaccgcgggaagtcagataacgttccatctgaggaggtcgttaagaagatgaagaactactggaggcagctcctgaacgccaagctgatcacgcaaaggaagttcgacaacctcaccaaggctgagagaggggggctctcagagctggacaaggctgggttcatcaagcggcagctggtcgagaccagacaaatcacgaagcacgttgcgcaaatcctcgactctcggatgaacacgaagtacgatgagaacgacaagctgatcagggaggtgaaggtcatcaccctgaagtctaagctcgtctccgacttccgcaaggacttccagttctacaaggttcgcgagatcaacaactaccaccatgcgcatgacgcttacctcaacgctgtggtcgggaccgccctgatcaagaagtacccgaagctggagagcgagttcgtgtacggggactacaaggtttacgatgtgcgcaagatgatcgccaagtcggagcaagagatcgggaaggccaccgccaagtacttcttctactcaaacatcatgaacttctttaagaccgagatcacgctggccaacggggagatccggaagagacccctcatcgagaccaacggggagacgggggagatcgtgtgggacaaggggagggatttcgcgaccgtccgcaaggttctctccatgccccaggtgaacatcgtcaagaagaccgaggtccaaacgggggggttctcaaaggagtctatcctgcctaagcggaacagcgacaagctcatcgccagaaagaaggactgggatccaaagaagtacggggggttcgacagccctaccgtcgcgtactcggttctggttgtggctaaggttgagaaggggaagtccaagaagctcaagagcgtgaaggagctcctggggatcaccatcatggagcgctccagcttcgagaagaacccaatcgacttcctggaggccaaggggtacaaggaggtgaagaaggacctgatcatcaagctcccgaagtactctctcttcgagctggagaacgggaggaagcgcatgctggcttccgctggggagctccaaaaggggaacgagctcgccctgccatccaagtatgtgaacttcctctacctggcgtcccactacgagaagctcaaggggagcccggaggacaacgagcaaaagcagctgttcgtcgagcagcacaagcattacctcgacgagatcatcgagcaaatctccgagttctccaaacgcgtgatcctcgcggacgctaacctggataaggtcctctccgcttacaacaagcaccgggacaagcccatcagagagcaggccgagaacatcatccatctcttcaccctgacgaacctcggggcccctgctgccttcaagtacttcgacaccacgatcgatcggaagagatacaccagcacgaaggaggtcctggacgcgaccctcatccaccaatctatcacggggctgtacgagacgagaatcgacctgtcacaactggggggggactccggcggctccagcggcggctccagcggcagcgagacgccgggcacgtccgagagcgccacccccgagagcagcggcggctccagcggcggctccagctccaagacccagcagccggagcccgtgaacatcagcacgaacaagatcgagaaccccctcgaggagatcgccatcctctcggagggcaggaggctctcggaggagaagctgttcatcacccagcagcgcatgcagaagacggaggagctcctggagaaggtgtgctccgagaacccgctcgaccccaacaagaccaagcagtggatgaaggcgagcatcaagctgtccgacccgagcaaggccatcaaggtcaagcccatgaagtactccccgatggacagggaggagttcgacaagcagatcaaggagctcctggacctcaaggtcatcaagccctccaagagcccccacatggctcccgcgttcctggtcaacaacgaggcggagaacggccgcggcaacaagaggatggtggtcaactacaaggccatgaacaaggccacggtgggcgacgcctacaacctccctaacaaggacgagctcctgacgctgatccgcggcaagaagatcttctccagcttcgactgcaagtccggcttctggcaggtcctgctcgaccaggagagcaggcccctcacggccttcacgtgcccccagggccactacgagtggaacgtggtcccattcggcctgaagcaggcccccagcatcttccagcgccacatggacgaggccttccgcgtgttcaggaagttctgctgcgtctacgtggacgacatcgtggtcttctccaacaacgaggaggaccacctcctgcacgtcgcgatgatcctccagaagtgcaaccagcacggcatcatcctctccaagaagaaggcccagctgttcaagaagaagatcaacttcctcggcctcgagatcgacgagggcacccacaagccacagggccacatcctcgagcacatcaacaagttcccggacaccctcgaggacaagaagcagctgcagcgcttcctcggcatcctgacgtacgcctccgactacatccccaacctggcccagatgaggcagcccctccaggccaagctgaaggagaacgtgccctggaagtggaccaaggaggacacgctctacatgcagaaggtcaagaagaacctgcagggcttcccgcccctccaccaccccctgcccgaggagaagctcatcatcgagacggacgcgagcgacgactactggggcggcatgctcaaggccatcaagatcaacgagggcaccaacacggagctgatctgccgctaccgctcgggcagcttcaaggcggccgagcgcaactaccactccaacgacaaggagacgctggccgtgatcaacacgatcaagaagttcagcatctacctcaccccggtccacttcctgatcaggaccgacaacacgcacttcaagtccttcgtcaacctcaactacaagggcgacagcaagctgggccgcaacatcaggtggcaggcgtggctctcccactacagcttcgacgtggagcacatcaagggcacggacaaccacttcgccgacttcctgtccagggagttcaacaaggtcaacagctccggcggcagcaagcgcacggcggacggctcggagttcgagccgaagaagaagcgcaaggtgtga

SEQ ID NO: 69 - DNA, 7224 bp, Artificial Sequence
  nCas9-KarmaRT
atggctcctaagaagaagcgcaaggttgggatccacggggttccggcggctatggacaagaagtacagcatcgggctcgacatcgggacgaacagcgttgggtgggcggtcatcacggacgagtacaaggttccctccaagaagttcaaggtgctggggaacaccgaccgccactccatcaagaagaacctcatcggggccctcctgttcgacagcggggagaccgctgaggctacgaggctcaagagaaccgcgaggcgcagatacacgagaaggaagaacaggatctgctacctccaagagatcttctccaacgagatggccaaggttgacgattcattcttccaccgcctggaggagtctttcctcgtggaggaggacaagaagcacgagcggcatcccatcttcgggaacatcgtcgacgaggttgcctaccacgagaagtaccctacgatctaccatctgcggaagaagctcgtggacagcaccgataaggcggacctcagactgatctacctcgccctggcgcacatgatcaagttccgcgggcatttcctgatcgagggggatctcaacccagacaactccgatgttgacaagctgttcatccaactcgtgcagacctacaaccaactcttcgaggagaacccgatcaacgcttcgggggtcgacgctaaggccatcctgtcagcccgcctctcgaagtcacgcaggctggagaacctgatcgcgcagctcccaggggagaagaagaacgggctgttcgggaacctcatcgctctctctctggggctcacgccgaacttcaagtccaacttcgatctcgccgaggacgcgaagctgcaactcagcaaggacacctacgacgatgacctcgataacctcctggcgcagatcggggatcaatacgctgacctgttcctcgccgcgaagaacctgtcggacgccatcctcctgtcagatatcctccgcgtgaacaccgagatcacgaaggccccactctctgcgtccatgatcaagcgctacgacgagcaccatcaggatctgaccctcctgaaggcgctggtccgccaacagctcccggagaagtacaaggagatcttcttcgatcagtcgaagaacgggtacgctgggtacatcgacgggggggcgtcacaagaggagttctacaagttcatcaagccaatcctggagaagatggacgggacggaggagctcctggtgaagctcaacagggaggacctcctgcggaagcagagaaccttcgataacgggtccatcccccaccaaatccatctcggggagctgcacgccatcctgagaaggcaagaggacttctaccctttcctcaaggataaccgggagaagatcgagaagatcctgaccttcagaatcccatactacgtcgggccgctcgctcgggggaacagcagattcgcctggatgacccgcaagtcggaggagaccatcacgccgtggaacttcgaggaggtggtcgacaagggggcgagcgctcagtcgttcatcgagaggatgaccaacttcgacaagaacctgcccaacgagaaggtgctccctaagcactcgctcctgtacgagtacttcaccgtctacaacgagctcacgaaggtgaagtatgtgaccgaggggatgagaaagcctgccttcctgtccggggagcagaagaaggcgatcgtggacctcctgttcaagaccaaccggaaggtcacggttaagcaactcaaggaggactacttcaagaagatcgagtgcttcgattcggtggagatctcaggggtcgaggacaggttcaacgcctccctcgggacctaccacgatctcctgaagatcatcaaggataaggacttcctggacaacgaggagaacgaggacatcctggaggacatcgtgctgaccctcacgctgttcgaggacagggagatgatcgaggagcgcctgaagacgtacgcccatctcttcgatgacaaggtcatgaagcaactcaagagaagaagatacaccgggtgggggaggctgtcccgcaagctcatcaacgggatccgggacaagcagtcagggaagaccatcctcgacttcctgaagtctgatgggttcgccaacaggaacttcatgcaactgatccacgatgactcactcaccttcaaggaggatatccaaaaggctcaggtgagcgggcagggggactcgctgcacgagcatatcgctaacctcgctgggtcccctgcgatcaagaaggggatcctgcagaccgtgaaggttgtggacgagctcgtgaaggtcatggggcggcataagcctgagaacatcgtcatcgagatggccagagagaaccaaaccacgcagaaggggcaaaagaactctagggagcgcatgaagcgcatcgaggaggggatcaaggagctggggtcccaaatcctcaaggagcacccagttgagaacacccaactgcagaacgagaagctctacctgtactacctccagaacgggagggatatgtatgtggaccaagagctggatatcaaccgcctcagcgattacgacgttgatgctatcgtgccccagtctttcctgaaggatgactccatcgacaacaaggtcctcaccaggtcggacaagaaccgcgggaagtcagataacgttccatctgaggaggtcgttaagaagatgaagaactactggaggcagctcctgaacgccaagctgatcacgcaaaggaagttcgacaacctcaccaaggctgagagaggggggctctcagagctggacaaggctgggttcatcaagcggcagctggtcgagaccagacaaatcacgaagcacgttgcgcaaatcctcgactctcggatgaacacgaagtacgatgagaacgacaagctgatcagggaggtgaaggtcatcaccctgaagtctaagctcgtctccgacttccgcaaggacttccagttctacaaggttcgcgagatcaacaactaccaccatgcgcatgacgcttacctcaacgctgtggtcgggaccgccctgatcaagaagtacccgaagctggagagcgagttcgtgtacggggactacaaggtttacgatgtgcgcaagatgatcgccaagtcggagcaagagatcgggaaggccaccgccaagtacttcttctactcaaacatcatgaacttctttaagaccgagatcacgctggccaacggggagatccggaagagacccctcatcgagaccaacggggagacgggggagatcgtgtgggacaaggggagggatttcgcgaccgtccgcaaggttctctccatgccccaggtgaacatcgtcaagaagaccgaggtccaaacgggggggttctcaaaggagtctatcctgcctaagcggaacagcgacaagctcatcgccagaaagaaggactgggatccaaagaagtacggggggttcgacagccctaccgtcgcgtactcggttctggttgtggctaaggttgagaaggggaagtccaagaagctcaagagcgtgaaggagctcctggggatcaccatcatggagcgctccagcttcgagaagaacccaatcgacttcctggaggccaaggggtacaaggaggtgaagaaggacctgatcatcaagctcccgaagtactctctcttcgagctggagaacgggaggaagcgcatgctggcttccgctggggagctccaaaaggggaacgagctcgccctgccatccaagtatgtgaacttcctctacctggcgtcccactacgagaagctcaaggggagcccggaggacaacgagcaaaagcagctgttcgtcgagcagcacaagcattacctcgacgagatcatcgagcaaatctccgagttctccaaacgcgtgatcctcgcggacgctaacctggataaggtcctctccgcttacaacaagcaccgggacaagcccatcagagagcaggccgagaacatcatccatctcttcaccctgacgaacctcggggcccctgctgccttcaagtacttcgacaccacgatcgatcggaagagatacaccagcacgaaggaggtcctggacgcgaccctcatccaccaatctatcacggggctgtacgagacgagaatcgacctgtcacaactggggggggactccggcggctccagcggcggctccagcggcagcgagacgccgggcacgtccgagagcgccacccccgagagcagcggcggctccagcggcggctccagcccgctcctgctcaccgccagcacgtccatccccaagagcaagaccttcaggttcgagaaccacctgctcctggaccgcgacttcctgccgtccgtcatgcacaggtggccccagcagctcaacatgaacgaccccgcggcctgcctggcggccaagctgaagcagacccgcagcatcatcaaggtctggatgaaggagcacaggaagcgctccagcctgaacgaggactgcaagttcacgatcgacctcctggactacatcgaggagctcaggcccctgaacgacggcgagaggtgcctccgcaacctggtgcaggagaagctgttccaggccatccagtccaaggtggcgtactggaagcagaggggcaaggtcaagcgcctcaagatcggcatcgacaacagcgccttcttcaaggcccacgcctccaagaactaccgcaacagcaggatccgctccatcaagcacaacggcgccgagatcgcggaccacaacggcaaggcgctcctgctccacagcttcttccagcacttcctgggctcggactgccagcccctctgggacttcgacctcagcaccctgtactccgaggacgagagggactacaacctgctcgccagccagggcctccagttctccaccgaggagatcatcacgagcatcaagggcatgaacagcaactcggctcccggcccggacggcttcggcccgagcttctacaactccgcgtggaacagcatcaaggaggacatcatcaagctggccgcgtccttctgcagcaactccgtgcagctggagaggatcaaccgcgcgcacatcgtcctcatcccgaagcccggcaaggagaacacggtggacggctacaggcccatctccctccagaactgcagcgtgaagatcctctccaaggtcctggccaacaggctccagaaggtcctgacccgcatgatccacctggaccagacgggcttcctcaagggccgctgcatcagcgagaacctgatctacgccacggagctcatccaggcttgccacgcgaggaagtgcaagacgctcatcatcaagctggacttcgccaaggcgttcgacagcgtcctctggacctccctgttccagatcctcgccgtgcgcggcttcccgaacaactggatctcctggatcaagagcctgctccagacgagcaagtccgccgtgctgctcaacggcatcccgggcaagtggatcaactgcaagaagggcctgaggcagggcgacccgctctccccctacctcttcatcctggtggccgacgtcctgcagaggctgctcgagaagaacttccagatccggcaccccatctaccaggacaggccatgcgctaccatccagtacgccgacgacaccctcgtgatctgcagggcggaggaggacgacgtcctggcgctgaggagcaccctgctccagttctccaaggccacgggcctccagatcaacttcgcgaagtccaccatgatcagcctgcacatcgaccgcagcaaggagtccagcctctccgagctgctccagtgcaagctcgagtcgctccccatgagctacctcggcctgccactcagcctgcacaagctgaccaacaacgacctccagccgatcgtggtcaaggtcgactccttcctgaccggctgggaggctagcctgctctcccaggcggagaggctcatcctggtgaacgcggtcctgtcctcggtgccggtctacgccatgagcgcgttcaagctcccgcccaaggtcatcgaggccatcgacaagaggaggcgcgcgttcttctggacgggcgacgacacctgctcgggcgctaagtgcctggtggcctgggacgaggtgtgcacggctaaggagaagggcggcctgggcatcaagagcctcaagacgcagaacgaggcgctgctcctgaagcgcctcttcaacctgttctccgacaactccagctggaccaactggatctggaaggagttcgacggcaggtccctcctcaagtccctcccgctgggccagcactggagcgtcttccagaagctcctgcccgagctgttcaagatcaccacggtgcacatcggcgacggcagcaggacgtccttctggcacgacaggtggacgggcaacatgaccctggctgcgcagttcgagagcctcttcagccactccacggacgacctggctaccgtggagcagatcctctccttcggcatctacgacatcctcaccccgcgcctctcgtcggcggcccagaacgacctccaggagctgcagacgatcctgcagaacttcagcctcaccaacgagtccgacacgaggctgaacaaccgcctcggcaacctgaccacgaagacgatctacgacctccgcagcccgcccggcttcccctcccccaactggaagttcatctgggactgcaggatgccgctgaagatcaagctgttcgcttggctcctggtcagggaccgcctgagcaccaagctcaacctcctcaagaagaagatcgtgcagaccgccacgtgcgacatctgcagcaccaccgacgagacggccgaccacctgagcttcaactgcccgttcgccatctccttctggcaggcgctccacatccagccgatcatcaacgagacgaagtacctcagccagctcaaggctcccgccaccatcccaacgcgccacttccagtccttcttcatgctgtgcttctgggtcctctggaaccacaggcacgacgtggtcttcaggggccgcccgcccagcatcgcttcctgcctccagaggggcatctcggagtcgtccctgtgggccgagacgttcacccccgacgacaggttcgtgatcgacgtctggaagggcgtgttctccagctccctccagacgctgcaccccatgtccggcggcagcaagcgcacggcggacggctcggagttcgagccgaagaagaagcgcaaggtgtga

SEQ ID NO: 70 - DNA, 6171 bp, Artificial Sequence
  nCas9-TnT1RT
atggctcctaagaagaagcgcaaggttgggatccacggggttccggcggctatggacaagaagtacagcatcgggctcgacatcgggacgaacagcgttgggtgggcggtcatcacggacgagtacaaggttccctccaagaagttcaaggtgctggggaacaccgaccgccactccatcaagaagaacctcatcggggccctcctgttcgacagcggggagaccgctgaggctacgaggctcaagagaaccgcgaggcgcagatacacgagaaggaagaacaggatctgctacctccaagagatcttctccaacgagatggccaaggttgacgattcattcttccaccgcctggaggagtctttcctcgtggaggaggacaagaagcacgagcggcatcccatcttcgggaacatcgtcgacgaggttgcctaccacgagaagtaccctacgatctaccatctgcggaagaagctcgtggacagcaccgataaggcggacctcagactgatctacctcgccctggcgcacatgatcaagttccgcgggcatttcctgatcgagggggatctcaacccagacaactccgatgttgacaagctgttcatccaactcgtgcagacctacaaccaactcttcgaggagaacccgatcaacgcttcgggggtcgacgctaaggccatcctgtcagcccgcctctcgaagtcacgcaggctggagaacctgatcgcgcagctcccaggggagaagaagaacgggctgttcgggaacctcatcgctctctctctggggctcacgccgaacttcaagtccaacttcgatctcgccgaggacgcgaagctgcaactcagcaaggacacctacgacgatgacctcgataacctcctggcgcagatcggggatcaatacgctgacctgttcctcgccgcgaagaacctgtcggacgccatcctcctgtcagatatcctccgcgtgaacaccgagatcacgaaggccccactctctgcgtccatgatcaagcgctacgacgagcaccatcaggatctgaccctcctgaaggcgctggtccgccaacagctcccggagaagtacaaggagatcttcttcgatcagtcgaagaacgggtacgctgggtacatcgacgggggggcgtcacaagaggagttctacaagttcatcaagccaatcctggagaagatggacgggacggaggagctcctggtgaagctcaacagggaggacctcctgcggaagcagagaaccttcgataacgggtccatcccccaccaaatccatctcggggagctgcacgccatcctgagaaggcaagaggacttctaccctttcctcaaggataaccgggagaagatcgagaagatcctgaccttcagaatcccatactacgtcgggccgctcgctcgggggaacagcagattcgcctggatgacccgcaagtcggaggagaccatcacgccgtggaacttcgaggaggtggtcgacaagggggcgagcgctcagtcgttcatcgagaggatgaccaacttcgacaagaacctgcccaacgagaaggtgctccctaagcactcgctcctgtacgagtacttcaccgtctacaacgagctcacgaaggtgaagtatgtgaccgaggggatgagaaagcctgccttcctgtccggggagcagaagaaggcgatcgtggacctcctgttcaagaccaaccggaaggtcacggttaagcaactcaaggaggactacttcaagaagatcgagtgcttcgattcggtggagatctcaggggtcgaggacaggttcaacgcctccctcgggacctaccacgatctcctgaagatcatcaaggataaggacttcctggacaacgaggagaacgaggacatcctggaggacatcgtgctgaccctcacgctgttcgaggacagggagatgatcgaggagcgcctgaagacgtacgcccatctcttcgatgacaaggtcatgaagcaactcaagagaagaagatacaccgggtgggggaggctgtcccgcaagctcatcaacgggatccgggacaagcagtcagggaagaccatcctcgacttcctgaagtctgatgggttcgccaacaggaacttcatgcaactgatccacgatgactcactcaccttcaaggaggatatccaaaaggctcaggtgagcgggcagggggactcgctgcacgagcatatcgctaacctcgctgggtcccctgcgatcaagaaggggatcctgcagaccgtgaaggttgtggacgagctcgtgaaggtcatggggcggcataagcctgagaacatcgtcatcgagatggccagagagaaccaaaccacgcagaaggggcaaaagaactctagggagcgcatgaagcgcatcgaggaggggatcaaggagctggggtcccaaatcctcaaggagcacccagttgagaacacccaactgcagaacgagaagctctacctgtactacctccagaacgggagggatatgtatgtggaccaagagctggatatcaaccgcctcagcgattacgacgttgatgctatcgtgccccagtctttcctgaaggatgactccatcgacaacaaggtcctcaccaggtcggacaagaaccgcgggaagtcagataacgttccatctgaggaggtcgttaagaagatgaagaactactggaggcagctcctgaacgccaagctgatcacgcaaaggaagttcgacaacctcaccaaggctgagagaggggggctctcagagctggacaaggctgggttcatcaagcggcagctggtcgagaccagacaaatcacgaagcacgttgcgcaaatcctcgactctcggatgaacacgaagtacgatgagaacgacaagctgatcagggaggtgaaggtcatcaccctgaagtctaagctcgtctccgacttccgcaaggacttccagttctacaaggttcgcgagatcaacaactaccaccatgcgcatgacgcttacctcaacgctgtggtcgggaccgccctgatcaagaagtacccgaagctggagagcgagttcgtgtacggggactacaaggtttacgatgtgcgcaagatgatcgccaagtcggagcaagagatcgggaaggccaccgccaagtacttcttctactcaaacatcatgaacttctttaagaccgagatcacgctggccaacggggagatccggaagagacccctcatcgagaccaacggggagacgggggagatcgtgtgggacaaggggagggatttcgcgaccgtccgcaaggttctctccatgccccaggtgaacatcgtcaagaagaccgaggtccaaacgggggggttctcaaaggagtctatcctgcctaagcggaacagcgacaagctcatcgccagaaagaaggactgggatccaaagaagtacggggggttcgacagccctaccgtcgcgtactcggttctggttgtggctaaggttgagaaggggaagtccaagaagctcaagagcgtgaaggagctcctggggatcaccatcatggagcgctccagcttcgagaagaacccaatcgacttcctggaggccaaggggtacaaggaggtgaagaaggacctgatcatcaagctcccgaagtactctctcttcgagctggagaacgggaggaagcgcatgctggcttccgctggggagctccaaaaggggaacgagctcgccctgccatccaagtatgtgaacttcctctacctggcgtcccactacgagaagctcaaggggagcccggaggacaacgagcaaaagcagctgttcgtcgagcagcacaagcattacctcgacgagatcatcgagcaaatctccgagttctccaaacgcgtgatcctcgcggacgctaacctggataaggtcctctccgcttacaacaagcaccgggacaagcccatcagagagcaggccgagaacatcatccatctcttcaccctgacgaacctcggggcccctgctgccttcaagtacttcgacaccacgatcgatcggaagagatacaccagcacgaaggaggtcctggacgcgaccctcatccaccaatctatcacggggctgtacgagacgagaatcgacctgtcacaactggggggggactccggcggctccagcggcggctccagcggcagcgagacgccgggcacgtccgagagcgccacccccgagagcagcggcggctccagcggcggctccagcgccgcggacatgtccgagaaggtgaagaacggcatcatcccgaacttcgtcaccatcccctccacgagcaacaaccccacctcggccgagtcgacgaccgacgaggtgagcgagcagggcgagcagccaggcgaggtcatcgagcagggcgagcagctggacgagggcgtggaggaggtggagcacccgacccagggcgaggagcagcaccagccactgaggaggtcggagcgcccgagggtggagtccaggaggtaccccagcaccgagtatgtgctcatctcggacgacagggagccggagtccctgaaggaggtgctcagccacccggagaagaaccagctcatgaaggcgatgcaggaggagatggagtccctgcagaagaacggcacctacaagctggtcgagctcccgaagggcaagaggcccctcaagtgcaagtgggtgttcaagctgaagaaggacggcgactgcaagctcgtccgctacaaggccaggctggtggtcaagggcttcgagcagaagaagggcatcgacttcgacgagatcttctcccccgtggtcaagatgaccagcatcaggaccatcctgtcgctggcggccagcctggacctcgaggtggagcagctggacgtcaagaccgccttcctgcacggcgacctcgaggaggagatctacatggagcagcccgagggcttcgaggtggccggcaagaagcacatggtctgcaagctgaacaagtcgctctacggcctcaagcaggccccgaggcagtggtacatgaagttcgactccttcatgaagtcccagacctacctcaagacgtactccgacccgtgcgtgtacttcaagaggttcagcgagaacaacttcatcatcctcctgctctacgtggacgacatgctcatcgtcggcaaggacaagggcctgatcgcgaagctcaagggcgacctgtccaagagcttcgacatgaaggacctcggcccggctcagcagatcctgggcatgaagatcgtgcgcgagaggacctccaggaagctgtggctcagccaggagaagtacatcgagcgcgtgctcgagaggttcaacatgaagaacgccaagccggtgtccacgcccctggctggccacctgaagctcagcaagaagatgtgccccaccacggtcgaggagaagggcaacatggctaaggtgccctactcctcggccgtgggctccctgatgtacgctatggtgtgcacccgccccgacatcgctcacgccgtgggcgtggtcagcaggttcctcgagaacccgggcaaggagcactgggaggcggtgaagtggatcctcaggtacctccggggcaccaccggcgactgcctctgcttcggtggctccgacccgatcctgaagggctacaccgacgcggacatggccggcgacatcgacaaccgcaagtccagcaccggctacctgttcacgttctccggcggcgccatctcctggcagagcaagctccagaagtgcgtggctctgagcacgaccgaggccgagtacatcgcggccacggagacgggcaaggagatgatctggctcaagcgcttcctgcaggagctgggcctccaccagaaggagtatgtggtctactgcgactcccagagcgcgatcgacctgtccaagaacagcatgtaccacgccaggaccaagcacatcgacgtgcgctaccactggatcagggagatggtggacgacgagtccctgaaggtcctcaagatcagcaccaacgagaaccccgccgacatgctcacgaaggtggtcccgaggaacaagttcgagctctgcaaggagctggtcggcatgcacagcaactccggcggcagcaagcgcacggcggacggctcggagttcgagccgaagaagaagcgcaaggtgtga

SEQ ID NO: 71 - DNA, 21 bp, Physcomitrella patens
gatgtcttaggccctgtgatt

SEQ ID NO: 72 - DNA, 443 bp, Artificial Sequence
  pEntPp-pegRNA-APT#1
aagcagacgtgttgcgacaggttagcgacgatgggtgtagatgtgatgtgatgtgatggtgtggttcttccacggcggcgtccttgcggtggcggagaaggggatatcccgaaggagcggcagcgggagagcacaagcagaaagggtgcagtgagtgagtgggtccagctgggtggctggccgagtggacgcgaccgggtttcgagggggcgggggagaaaagggatggagcgagggatataacccacatggaatggaggtgggtgtgaaggcgggtatataggaaggtggaggacttacaacccatgatgtcttaggccctgtgattgttttagagctagaaatagcaagttaaaataaggctagtccgttatcaacttgaaaaagtggcaccgagtcggtgctactacactcttagctaatcacagggcctaagttttttt

SEQ ID NO: 73 - DNA, 20 bp, Physcomitrella patens
ggtgaagatgtcggcctcca

SEQ ID NO: 74 - DNA, 442 bp, Artificial Sequence
  pEntPp-pegRNA-APT#2
aagcagacgtgttgcgacaggttagcgacgatgggtgtagatgtgatgtgatgtgatggtgtggttcttccacggcggcgtccttgcggtggcggagaaggggatatcccgaaggagcggcagcgggagagcacaagcagaaagggtgcagtgagtgagtgggtccagctgggtggctggccgagtggacgcgaccgggtttcgagggggcgggggagaaaagggatggagcgagggatataacccacatggaatggaggtgggtgtgaaggcgggtatataggaaggtggaggacttacaacccatggtgaagatgtcggcctccagttttagagctagaaatagcaagttaaaataaggctagtccgttatcaacttgaaaaagtggcaccgagtcggtgcgtcgccatccttggtatgaggccgacatcttcttttttt

SEQ ID NO: 75 - DNA, 20 bp, Physcomitrella patens
tgtcttaggccctgtgatta

SEQ ID NO: 76 - DNA, 449 bp, Artificial Sequence
  pEntPp-pegRNA-APT#3
aagcagacgtgttgcgacaggttagcgacgatgggtgtagatgtgatgtgatgtgatggtgtggttcttccacggcggcgtccttgcggtggcggagaaggggatatcccgaaggagcggcagcgggagagcacaagcagaaagggtgcagtgagtgagtgggtccagctgggtggctggccgagtggacgcgaccgggtttcgagggggcgggggagaaaagggatggagcgagggatataacccacatggaatggaggtgggtgtgaaggcgggtatataggaaggtggaggacttacaacccattgtcttaggccctgtgattagtttaagagctatgctggaaacagcatagcaagtttaaataaggctagtccgttatcaacttgaaaaagtggcaccgagtcggtgctatactcttctcaaatcacagggcctaagttttttt

SEQ ID NO: 77 - DNA, 20 bp, Physcomitrella patens
gatgacttaattgccaccgg

SEQ ID NO: 78 - DNA, 449 bp, Artificial Sequence
  pEntPp-pegRNA-APT#6
aagcagacgtgttgcgacaggttagcgacgatgggtgtagatgtgatgtgatgtgatggtgtggttcttccacggcggcgtccttgcggtggcggagaaggggatatcccgaaggagcggcagcgggagagcacaagcagaaagggtgcagtgagtgagtgggtccagctgggtggctggccgagtggacgcgaccgggtttcgagggggcgggggagaaaagggatggagcgagggatataacccacatggaatggaggtgggtgtgaaggcgggtatataggaaggtggaggacttacaacccatgatgacttaattgccaccgggtttaagagctatgctggaaacagcatagcaagtttaaataaggctagtccgttatcaacttgaaaaagtggcaccgagtcggtgctccaagagttcatcaggtggcaattaagtttttttt

SEQ ID NO: 79 - DNA, 307 bp, Physcomitrella patens
aagcagacgtgttgcgacaggttagcgacgatgggtgtagatgtgatgtgatgtgatggtgtggttcttccacggcggcgtccttgcggtggcggagaaggggatatcccgaaggagcggcagcgggagagcacaagcagaaagggtgcagtgagtgagtgggtgcagctgggtggctggccgagtggacgcgaccgggtttcgagggggcgggggagaaaagggatggagcgagggatataacccacatggaatggaggtgggtgtgaaggcgggtatataggaaggtggaggacttacaacccat

SEQ ID NO: 80 - DNA, 20 bp, Physcomitrella patens
acgtcagcacaaaattgtca

SEQ ID NO: 81 - DNA, 482 bp, Artificial Sequence
  pEntPp-gRNA-APT#3
tctatattagatgattgttcatgtagaacacaaggaagaaatacaaaacgtgtccatgcacataagatctagagtattggataaaataatacgtatagatgtatagatgagcaagtagtaaaagatgacttactcctagttaaattaagcatggaaagaaagtgttagcatgtacaaaagagaaataaaaataaaaacacaaaacctctgtatcgatagatttcttagaaggggaacgaaacataaaacttcaagtgaatgcaagtgatggatggatactattgagtgagtgcggggcaggggaggtggagttgcaccatagccgttgcggtggaaggggaaggccggtatatggaggtgggggtggagccggtctggcacgtcagcacaaaattgtcagttttagagctagaaatagcaagttaaaataaggctagtccgttatcaacttgaaaaagtggcaccgagtcggtgcttttttt

SEQ ID NO: 82 - DNA, 315 bp, Physcomitrella patens
agatctagagtattggataaaataatacgtatagatgtatagatgagcaagtagtaaaagatgacttactcctagttaaattaagcatggaaagaaagtgttagcatgtacaaaagagaaataaaaataaaaacacaaaacctctgtatcgatagatttcttagaaggggaacgaaacataaaacttcaagtgaatgcaagtgatggatggatactattgagtgagtgcggggcaggggaggtggagttgcaccatagccgttgcggtggaaggggaaggccggtatatggaggtgggggtggagccggtctggc

SEQ ID NO: 83 - DNA, 20 bp, Physcomitrella patens
ggatgctgtccgagatatac

SEQ ID NO: 84 - DNA, 410 bp, Artificial Sequence
  pEntPp-gRNA-APT#5
aagcagacgtgttgcgacaggttagcgacgatgggtgtagatgtgatgtgatgtgatggtgtggttcttccacggcggcgtccttgcggtggcggagaaggggatatcccgaaggagcggcagcgggagagcacaagcagaaagggtgcagtgagtgagtgggtccagctgggtggctggccgagtggacgcgaccgggtttcgagggggggggggagaaaagggatggagcgagggatataacccacatggaatggaggtgggtgtgaaggcgggtatataggaaggtggaggacttacaacccatggatgctgtccgagatatacgttttagagctagaaatagcaagttaaaataaggctagtccgttatcaacttgaaaaagtggcaccgagtcggtgcttttttt

SEQ ID NO: 85 - protein, 464 aa, Nicotiana tabacum
AADMSEKVKNGIIPNFVTIPSTSNNPTSAESTTDEVSEQGEQPGEVIEQGEQLDEGVEEVEHPTQGEEQHQPLRRSERPRVESRRYPSTEYVLISDDREPESLKEVLSHPEKNQLMKAMQEEMESLQKNGTYKLVELPKGKRPLKCKWVFKLKKDGDCKLVRYKARLVVKGFEQKKGIDFDEIFSPVVKMTSIRTILSLAASLDLEVEQLDVKTAFLHGDLEEEIYMEQPEGFEVAGKKHMVCKLNKSLYGLKQAPRQWYMKFDSFMKSQTYLKTYSDPCVYFKRFSENNFIILLLYVDDMLIVGKDKGLIAKLKGDLSKSFDMKDLGPAQQILGMKIVRERTSRKLWLSQEKYIERVLERFNMKNAKPVSTPLAGHLKLSKKMCPTTVEEKGNMAKVPYSSAVGSLMYAMVCTRPDIAHAVGVVSRFLENPGKEHWEAVKWILRYLRGTTGDCLCFGGSDPIL

SEQ ID NO: 86 - DNA, 5712 bp, Artificial Sequence
  nCas9-TnT1RTv2
atggctcctaagaagaagcgcaaggttgggatccacggggttccggcggctatggacaagaagtacagcatcgggctcgacatcgggacgaacagcgttgggtgggcggtcatcacggacgagtacaaggttccctccaagaagttcaaggtgctggggaacaccgaccgccactccatcaagaagaacctcatcggggccctcctgttcgacagcggggagaccgctgaggctacgaggctcaagagaaccgcgaggcgcagatacacgagaaggaagaacaggatctgctacctccaagagatcttctccaacgagatggccaaggttgacgattcattcttccaccgcctggaggagtctttcctcgtggaggaggacaagaagcacgagcggcatcccatcttcgggaacatcgtcgacgaggttgcctaccacgagaagtaccctacgatctaccatctgcggaagaagctcgtggacagcaccgataaggcggacctcagactgatctacctcgccctggcgcacatgatcaagttccgcgggcatttcctgatcgagggggatctcaacccagacaactccgatgttgacaagctgttcatccaactcgtgcagacctacaaccaactcttcgaggagaacccgatcaacgcttcgggggtcgacgctaaggccatcctgtcagcccgcctctcgaagtcacgcaggctggagaacctgatcgcgcagctcccaggggagaagaagaacgggctgttcgggaacctcatcgctctctctctggggctcacgccgaacttcaagtccaacttcgatctcgccgaggacgcgaagctgcaactcagcaaggacacctacgacgatgacctcgataacctcctggcgcagatcggggatcaatacgctgacctgttcctcgccgcgaagaacctgtcggacgccatcctcctgtcagatatcctccgcgtgaacaccgagatcacgaaggccccactctctgcgtccatgatcaagcgctacgacgagcaccatcaggatctgaccctcctgaaggcgctggtccgccaacagctcccggagaagtacaaggagatcttcttcgatcagtcgaagaacgggtacgctgggtacatcgacgggggggcgtcacaagaggagttctacaagttcatcaagccaatcctggagaagatggacgggacggaggagctcctggtgaagctcaacagggaggacctcctgcggaagcagagaaccttcgataacgggtccatcccccaccaaatccatctcggggagctgcacgccatcctgagaaggcaagaggacttctaccctttcctcaaggataaccgggagaagatcgagaagatcctgaccttcagaatcccatactacgtcgggccgctcgctcgggggaacagcagattcgcctggatgacccgcaagtcggaggagaccatcacgccgtggaacttcgaggaggtggtcgacaagggggcgagcgctcagtcgttcatcgagaggatgaccaacttcgacaagaacctgcccaacgagaaggtgctccctaagcactcgctcctgtacgagtacttcaccgtctacaacgagctcacgaaggtgaagtatgtgaccgaggggatgagaaagcctgccttcctgtccggggagcagaagaaggcgatcgtggacctcctgttcaagaccaaccggaaggtcacggttaagcaactcaaggaggactacttcaagaagatcgagtgcttcgattcggtggagatctcaggggtcgaggacaggttcaacgcctccctcgggacctaccacgatctcctgaagatcatcaaggataaggacttcctggacaacgaggagaacgaggacatcctggaggacatcgtgctgaccctcacgctgttcgaggacagggagatgatcgaggagcgcctgaagacgtacgcccatctcttcgatgacaaggtcatgaagcaactcaagagaagaagatacaccgggtgggggaggctgtcccgcaagctcatcaacgggatccgggacaagcagtcagggaagaccatcctcgacttcctgaagtctgatgggttcgccaacaggaacttcatgcaactgatccacgatgactcactcaccttcaaggaggatatccaaaaggctcaggtgagcgggcagggggactcgctgcacgagcatatcgctaacctcgctgggtcccctgcgatcaagaaggggatcctgcagaccgtgaaggttgtggacgagctcgtgaaggtcatggggcggcataagcctgagaacatcgtcatcgagatggccagagagaaccaaaccacgcagaaggggcaaaagaactctagggagcgcatgaagcgcatcgaggaggggatcaaggagctggggtcccaaatcctcaaggagcacccagttgagaacacccaactgcagaacgagaagctctacctgtactacctccagaacgggagggatatgtatgtggaccaagagctggatatcaaccgcctcagcgattacgacgttgatgctatcgtgccccagtctttcctgaaggatgactccatcgacaacaaggtcctcaccaggtcggacaagaaccgcgggaagtcagataacgttccatctgaggaggtcgttaagaagatgaagaactactggaggcagctcctgaacgccaagctgatcacgcaaaggaagttcgacaacctcaccaaggctgagagaggggggctctcagagctggacaaggctgggttcatcaagcggcagctggtcgagaccagacaaatcacgaagcacgttgcgcaaatcctcgactctcggatgaacacgaagtacgatgagaacgacaagctgatcagggaggtgaaggtcatcaccctgaagtctaagctcgtctccgacttccgcaaggacttccagttctacaaggttcgcgagatcaacaactaccaccatgcgcatgacgcttacctcaacgctgtggtcgggaccgccctgatcaagaagtacccgaagctggagagcgagttcgtgtacggggactacaaggtttacgatgtgcgcaagatgatcgccaagtcggagcaagagatcgggaaggccaccgccaagtacttcttctactcaaacatcatgaacttctttaagaccgagatcacgctggccaacggggagatccggaagagacccctcatcgagaccaacggggagacgggggagatcgtgtgggacaaggggagggatttcgcgaccgtccgcaaggttctctccatgccccaggtgaacatcgtcaagaagaccgaggtccaaacgggggggttctcaaaggagtctatcctgcctaagcggaacagcgacaagctcatcgccagaaagaaggactgggatccaaagaagtacggggggttcgacagccctaccgtcgcgtactcggttctggttgtggctaaggttgagaaggggaagtccaagaagctcaagagcgtgaaggagctcctggggatcaccatcatggagcgctccagcttcgagaagaacccaatcgacttcctggaggccaaggggtacaaggaggtgaagaaggacctgatcatcaagctcccgaagtactctctcttcgagctggagaacgggaggaagcgcatgctggcttccgctggggagctccaaaaggggaacgagctcgccctgccatccaagtatgtgaacttcctctacctggcgtcccactacgagaagctcaaggggagcccggaggacaacgagcaaaagcagctgttcgtcgagcagcacaagcattacctcgacgagatcatcgagcaaatctccgagttctccaaacgcgtgatcctcgcggacgctaacctggataaggtcctctccgcttacaacaagcaccgggacaagcccatcagagagcaggccgagaacatcatccatctcttcaccctgacgaacctcggggcccctgctgccttcaagtacttcgacaccacgatcgatcggaagagatacaccagcacgaaggaggtcctggacgcgaccctcatccaccaatctatcacggggctgtacgagacgagaatcgacctgtcacaactggggggggactccggcggctccagcggcggctccagcggcagcgagacgccgggcacgtccgagagcgccacccccgagagcagcggcggctccagcggcggctccagcgccgcggacatgtccgagaaggtgaagaacggcatcatcccgaacttcgtcaccatcccctccacgagcaacaaccccacctcggccgagtcgacgaccgacgaggtgagcgagcagggcgagcagccaggcgaggtcatcgagcagggcgagcagctggacgagggcgtggaggaggtggagcacccgacccagggcgaggagcagcaccagccactgaggaggtcggagcgcccgagggtggagtccaggaggtaccccagcaccgagtatgtgctcatctcggacgacagggagccggagtccctgaaggaggtgctcagccacccggagaagaaccagctcatgaaggcgatgcaggaggagatggagtccctgcagaagaacggcacctacaagctggtcgagctcccgaagggcaagaggcccctcaagtgcaagtgggtgttcaagctgaagaaggacggcgactgcaagctcgtccgctacaaggccaggctggtggtcaagggcttcgagcagaagaagggcatcgacttcgacgagatcttctcccccgtggtcaagatgaccagcatcaggaccatcctgtcgctggcggccagcctggacctcgaggtggagcagctggacgtcaagaccgccttcctgcacggcgacctcgaggaggagatctacatggagcagcccgagggcttcgaggtggccggcaagaagcacatggtctgcaagctgaacaagtcgctctacggcctcaagcaggccccgaggcagtggtacatgaagttcgactccttcatgaagtcccagacctacctcaagacgtactccgacccgtgcgtgtacttcaagaggttcagcgagaacaacttcatcatcctcctgctctacgtggacgacatgctcatcgtcggcaaggacaagggcctgatcgcgaagctcaagggcgacctgtccaagagcttcgacatgaaggacctcggcccggctcagcagatcctgggcatgaagatcgtgcgcgagaggacctccaggaagctgtggctcagccaggagaagtacatcgagcgcgtgctcgagaggttcaacatgaagaacgccaagccggtgtccacgcccctggctggccacctgaagctcagcaagaagatgtgccccaccacggtcgaggagaagggcaacatggctaaggtgccctactcctcggccgtgggctccctgatgtacgctatggtgtgcacccgccccgacatcgctcacgccgtgggcgtggtcagcaggttcctcgagaacccgggcaaggagcactgggaggcggtgaagtggatcctcaggtacctccggggcaccaccggcgactgcctctgcttcggtggctccgacccgatcctgtccggcggcagcaagcgcacggcggacggctcggagttcgagccgaagaagaagcgcaaggtgtga

SEQ ID NO: 87 - protein, 617 aa, Nicotiana tabacum
AADMSEKVKNGIIPNFVTIPSTSNNPTSAESTTDEVSEQGEQPGEVIEQGEQLDEGVEEVEHPTQGEEQHQPLRRSERPRVESRRYPSTEYVLISDDREPESLKEVLSHPEKNQLMKAMQEEMESLQKNGTYKLVELPKGKRPLKCKWVFKLKKDGDCKLVRYKARLVVKGFEQKKGIDFDEIFSPVVKMTSIRTILSLAASLDLEVEQLDVKTAFLHGDLEEEIYMEQPEGFEVAGKKHMVCKLNKSLYGLKQAPRQWYMKFDSFMKSQTYLKTYSDPCVYFKRFSENNFIILLLYVDDMLIVGKDKGLIAKLKGDLSKSFDMKDLGPAQQILGMKIVRERTSRKLWLSQEKYIERVLERFNMKNAKPVSTPLAGHLKLSKKMCPTTVEEKGNMAKVPYSSAVGSLMYAMVCTRPDIAHAVGVVSRFLENPGKEHWEAVKWILRYLRGTTGDCLCFGGSDPILKGYTNADMAGDIDNRKSSTGYLFTFSGGAISWQSKLQKCVALSTTEAEYIAATETGKEMIWLKRFLQELGLHQKEYVVYCDSQSAIDLSKNSMYHARTKHIDVRYHWIREMVDDESLKVLKISTNENPADMLTKVVPRNKFELCKELVGMHSN

SEQ ID NO: 88 - DNA, 6171 bp, Artificial Sequence
  nCas9-TnT1(D469N)RT
atggctcctaagaagaagcgcaaggttgggatccacggggttccggcggctatggacaagaagtacagcatcgggctcgacatcgggacgaacagcgttgggtgggcggtcatcacggacgagtacaaggttccctccaagaagttcaaggtgctggggaacaccgaccgccactccatcaagaagaacctcatcggggccctcctgttcgacagcggggagaccgctgaggctacgaggctcaagagaaccgcgaggcgcagatacacgagaaggaagaacaggatctgctacctccaagagatcttctccaacgagatggccaaggttgacgattcattcttccaccgcctggaggagtctttcctcgtggaggaggacaagaagcacgagcggcatcccatcttcgggaacatcgtcgacgaggttgcctaccacgagaagtaccctacgatctaccatctgcggaagaagctcgtggacagcaccgataaggcggacctcagactgatctacctcgccctggcgcacatgatcaagttccgcgggcatttcctgatcgagggggatctcaacccagacaactccgatgttgacaagctgttcatccaactcgtgcagacctacaaccaactcttcgaggagaacccgatcaacgcttcgggggtcgacgctaaggccatcctgtcagcccgcctctcgaagtcacgcaggctggagaacctgatcgcgcagctcccaggggagaagaagaacgggctgttcgggaacctcatcgctctctctctggggctcacgccgaacttcaagtccaacttcgatctcgccgaggacgcgaagctgcaactcagcaaggacacctacgacgatgacctcgataacctcctggcgcagatcggggatcaatacgctgacctgttcctcgccgcgaagaacctgtcggacgccatcctcctgtcagatatcctccgcgtgaacaccgagatcacgaaggccccactctctgcgtccatgatcaagcgctacgacgagcaccatcaggatctgaccctcctgaaggcgctggtccgccaacagctcccggagaagtacaaggagatcttcttcgatcagtcgaagaacgggtacgctgggtacatcgacgggggggcgtcacaagaggagttctacaagttcatcaagccaatcctggagaagatggacgggacggaggagctcctggtgaagctcaacagggaggacctcctgcggaagcagagaaccttcgataacgggtccatcccccaccaaatccatctcggggagctgcacgccatcctgagaaggcaagaggacttctaccctttcctcaaggataaccgggagaagatcgagaagatcctgaccttcagaatcccatactacgtcgggccgctcgctcgggggaacagcagattcgcctggatgacccgcaagtcggaggagaccatcacgccgtggaacttcgaggaggtggtcgacaagggggcgagcgctcagtcgttcatcgagaggatgaccaacttcgacaagaacctgcccaacgagaaggtgctccctaagcactcgctcctgtacgagtacttcaccgtctacaacgagctcacgaaggtgaagtatgtgaccgaggggatgagaaagcctgccttcctgtccggggagcagaagaaggcgatcgtggacctcctgttcaagaccaaccggaaggtcacggttaagcaactcaaggaggactacttcaagaagatcgagtgcttcgattcggtggagatctcaggggtcgaggacaggttcaacgcctccctcgggacctaccacgatctcctgaagatcatcaaggataaggacttcctggacaacgaggagaacgaggacatcctggaggacatcgtgctgaccctcacgctgttcgaggacagggagatgatcgaggagcgcctgaagacgtacgcccatctcttcgatgacaaggtcatgaagcaactcaagagaagaagatacaccgggtgggggaggctgtcccgcaagctcatcaacgggatccgggacaagcagtcagggaagaccatcctcgacttcctgaagtctgatgggttcgccaacaggaacttcatgcaactgatccacgatgactcactcaccttcaaggaggatatccaaaaggctcaggtgagcgggcagggggactcgctgcacgagcatatcgctaacctcgctgggtcccctgcgatcaagaaggggatcctgcagaccgtgaaggttgtggacgagctcgtgaaggtcatggggcggcataagcctgagaacatcgtcatcgagatggccagagagaaccaaaccacgcagaaggggcaaaagaactctagggagcgcatgaagcgcatcgaggaggggatcaaggagctggggtcccaaatcctcaaggagcacccagttgagaacacccaactgcagaacgagaagctctacctgtactacctccagaacgggagggatatgtatgtggaccaagagctggatatcaaccgcctcagcgattacgacgttgatgctatcgtgccccagtctttcctgaaggatgactccatcgacaacaaggtcctcaccaggtcggacaagaaccgcgggaagtcagataacgttccatctgaggaggtcgttaagaagatgaagaactactggaggcagctcctgaacgccaagctgatcacgcaaaggaagttcgacaacctcaccaaggctgagagaggggggctctcagagctggacaaggctgggttcatcaagcggcagctggtcgagaccagacaaatcacgaagcacgttgcgcaaatcctcgactctcggatgaacacgaagtacgatgagaacgacaagctgatcagggaggtgaaggtcatcaccctgaagtctaagctcgtctccgacttccgcaaggacttccagttctacaaggttcgcgagatcaacaactaccaccatgcgcatgacgcttacctcaacgctgtggtcgggaccgccctgatcaagaagtacccgaagctggagagcgagttcgtgtacggggactacaaggtttacgatgtgcgcaagatgatcgccaagtcggagcaagagatcgggaaggccaccgccaagtacttcttctactcaaacatcatgaacttctttaagaccgagatcacgctggccaacggggagatccggaagagacccctcatcgagaccaacggggagacgggggagatcgtgtgggacaaggggagggatttcgcgaccgtccgcaaggttctctccatgccccaggtgaacatcgtcaagaagaccgaggtccaaacgggggggttctcaaaggagtctatcctgcctaagcggaacagcgacaagctcatcgccagaaagaaggactgggatccaaagaagtacggggggttcgacagccctaccgtcgcgtactcggttctggttgtggctaaggttgagaaggggaagtccaagaagctcaagagcgtgaaggagctcctggggatcaccatcatggagcgctccagcttcgagaagaacccaatcgacttcctggaggccaaggggtacaaggaggtgaagaaggacctgatcatcaagctcccgaagtactctctcttcgagctggagaacgggaggaagcgcatgctggcttccgctggggagctccaaaaggggaacgagctcgccctgccatccaagtatgtgaacttcctctacctggcgtcccactacgagaagctcaaggggagcccggaggacaacgagcaaaagcagctgttcgtcgagcagcacaagcattacctcgacgagatcatcgagcaaatctccgagttctccaaacgcgtgatcctcgcggacgctaacctggataaggtcctctccgcttacaacaagcaccgggacaagcccatcagagagcaggccgagaacatcatccatctcttcaccctgacgaacctcggggcccctgctgccttcaagtacttcgacaccacgatcgatcggaagagatacaccagcacgaaggaggtcctggacgcgaccctcatccaccaatctatcacggggctgtacgagacgagaatcgacctgtcacaactggggggggactccggcggctccagcggcggctccagcggcagcgagacgccgggcacgtccgagagcgccacccccgagagcagcggcggctccagcggcggctccagcgccgcggacatgtccgagaaggtgaagaacggcatcatcccgaacttcgtcaccatcccctccacgagcaacaaccccacctcggccgagtcgacgaccgacgaggtgagcgagcagggcgagcagccaggcgaggtcatcgagcagggcgagcagctggacgagggcgtggaggaggtggagcacccgacccagggcgaggagcagcaccagccactgaggaggtcggagcgcccgagggtggagtccaggaggtaccccagcaccgagtatgtgctcatctcggacgacagggagccggagtccctgaaggaggtgctcagccacccggagaagaaccagctcatgaaggcgatgcaggaggagatggagtccctgcagaagaacggcacctacaagctggtcgagctcccgaagggcaagaggcccctcaagtgcaagtgggtgttcaagctgaagaaggacggcgactgcaagctcgtccgctacaaggccaggctggtggtcaagggcttcgagcagaagaagggcatcgacttcgacgagatcttctcccccgtggtcaagatgaccagcatcaggaccatcctgtcgctggcggccagcctggacctcgaggtggagcagctggacgtcaagaccgccttcctgcacggcgacctcgaggaggagatctacatggagcagcccgagggcttcgaggtggccggcaagaagcacatggtctgcaagctgaacaagtcgctctacggcctcaagcaggccccgaggcagtggtacatgaagttcgactccttcatgaagtcccagacctacctcaagacgtactccgacccgtgcgtgtacttcaagaggttcagcgagaacaacttcatcatcctcctgctctacgtggacgacatgctcatcgtcggcaaggacaagggcctgatcgcgaagctcaagggcgacctgtccaagagcttcgacatgaaggacctcggcccggctcagcagatcctgggcatgaagatcgtgcgcgagaggacctccaggaagctgtggctcagccaggagaagtacatcgagcgcgtgctcgagaggttcaacatgaagaacgccaagccggtgtccacgcccctggctggccacctgaagctcagcaagaagatgtgccccaccacggtcgaggagaagggcaacatggctaaggtgccctactcctcggccgtgggctccctgatgtacgctatggtgtgcacccgccccgacatcgctcacgccgtgggcgtggtcagcaggttcctcgagaacccgggcaaggagcactgggaggcggtgaagtggatcctcaggtacctccggggcaccaccggcgactgcctctgcttcggtggctccgacccgatcctgaagggctacaccaacgcggacatggccggcgacatcgacaaccgcaagtccagcaccggctacctgttcacgttctccggcggcgccatctcctggcagagcaagctccagaagtgcgtggctctgagcacgaccgaggccgagtacatcgcggccacggagacgggcaaggagatgatctggctcaagcgcttcctgcaggagctgggcctccaccagaaggagtatgtggtctactgcgactcccagagcgcgatcgacctgtccaagaacagcatgtaccacgccaggaccaagcacatcgacgtgcgctaccactggatcagggagatggtggacgacgagtccctgaaggtcctcaagatcagcaccaacgagaaccccgccgacatgctcacgaaggtggtcccgaggaacaagttcgagctctgcaaggagctggtcggcatgcacagcaactccggcggcagcaagcgcacggcggacggctcggagttcgagccgaagaagaagcgcaaggtgtga

SEQ ID NO: 89 - DNA, 720 bp, Artificial Sequence
  BMPmm
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccacccttttgaccaccttcacccacggcgtgcagtgcttcggccgctaccccgaccacatgaagcagcacgacttcttcaagtccgccatgcccgaaggctacgtccaggagcgcaccatcagcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaaacagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 90 - protein, 239 aa, Artificial Sequence
  BMPmm
MVSKGEELFTGVVPILVELDGDVNGHKFSVSGEGEGDATYGKLTLKFICTTGKLPVPWPTLLTTFTHGVQCFGRYPDHMKQHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITLGMDELYK

SEQ ID NO: 91 - DNA, 720 bp, Artificial Sequence
  GFPmm
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccacccttttgaccaccttcacctacggcgtgcagtgcttcggccgctaccccgaccacatgaagcagcacgacttcttcaagtccgccatgcccgaaggctacgtccaggagcgcaccatcagcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaaacagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 92 - protein, 239 aa, Artificial Sequence
  GFPmm
MVSKGEELFTGVVPILVELDGDVNGHKFSVSGEGEGDATYGKLTLKFICTTGKLPVPWPTLLTTFTYGVQCFGRYPDHMKQHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITLGMDELYK

SEQ ID NO: 93 - DNA, 20 bp, Artificial Sequence
  pegRNA-BFP-01 Target
cttttgaccaccttcaccca

SEQ ID NO: 94 - DNA, 123 bp, Artificial Sequence
  pegRNA-BFP-01
cttttgaccaccttcacccagttttagagctagaaatagcaagttaaaataaggctagtccgttatcaacttgaaaaagtggcaccgagtcggtgcactgcacgccgtaggtgaaggtggtca

SEQ ID NO: 95 - DNA, 20 bp, Artificial Sequence
  gRNA-BFP-R3 Target
ggtggtgcagatgaacttca

SEQ ID NO: 96 - DNA, 96 bp, Artificial Sequence
  gRNA-BFP-R3
ggtggtgcagatgaacttcagtttaagagctagaaatagcaagtttaaataaggctagtccgttatcaacttgaaaaagtggcaccgagtcggtgc

SEQ ID NO: 97 - DNA, 19 bp, Artificial Sequence
  gRNA-BFP-PE3b Target
cactgcacgccgtaggtga

SEQ ID NO: 98 - DNA, 95 bp, Artificial Sequence
  gRNA-PE3b-01
cactgcacgccgtaggtgagttttagagctagaaatagcaagttaaaataaggctagtccgttatcaacttgaaaaagtggcaccgagtcggtgc

SEQ ID NO: 99 - DNA, 238 bp, Artificial Sequence
  BFP_SpCas9_pegRNA_RZ_01
caaaagctgatgagtccgtgaggacgaaacgagtaagctcgtccttttgaccaccttcacccagttttagagctagaaatagcaagttaaaataaggctagtccgttatcaacttgaaaaagtggcaccgagtcggtgcactgcacgccgtaggtgaaggtggtcattttggccggcatggtcccagcctcctcgctggcgccggctgggcaacatgcttcggcatggcgaatgggac

SEQ ID NO: 100 - DNA, 211 bp, Artificial Sequence
  BFP_SpCas9_gRNA_RZ_R3
accaccctgatgagtccgtgaggacgaaacgagtaagctcgtcggtggtgcagatgaacttcagtttaagagctagaaatagcaagtttaaataaggctagtccgttatcaacttgaaaaagtggcaccgagtcggtgcttttggccggcatggtcccagcctcctcgctggcgccggctgggcaacatgcttcggcatggcgaatgggac

SEQ ID NO: 101 - DNA, 210 bp, Artificial Sequence
  BFP_SpCas9_gRNA_RZ_PE3b
gcagtgctgatgagtccgtgaggacgaaacgagtaagctcgtccactgcacgccgtaggtgagttttagagctagaaatagcaagttaaaataaggctagtccgttatcaacttgaaaaagtggcaccgagtcggtgcttttggccggcatggtcccagcctcctcgctggcgccggctgggcaacatgcttcggcatggcgaatgggac

SEQ ID NO: 102 - DNA, 42 bp, Artificial Sequence
  SV40 NLS
atggctcctaagaagaagcgcaaggttgggatccacggggtt

SEQ ID NO: 103 - DNA, 48 bp, Artificial Sequence
  XlNucleoplasmin NLS
aagaggccggcggcaaccaagaaggcgggccaggccaagaagaagaag

SEQ ID NO: 104 - protein, 1903 aa, Artificial Sequence
  nCas9-Tnt1_RTv2
MAPKKKRKVGIHGVPAAMDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSAADMSEKVKNGIIPNFVTIPSTSNNPTSAESTTDEVSEQGEQPGEVIEQGEQLDEGVEEVEHPTQGEEQHQPLRRSERPRVESRRYPSTEYVLISDDREPESLKEVLSHPEKNQLMKAMQEEMESLQKNGTYKLVELPKGKRPLKCKWVFKLKKDGDCKLVRYKARLVVKGFEQKKGIDFDEIFSPVVKMTSIRTILSLAASLDLEVEQLDVKTAFLHGDLEEEIYMEQPEGFEVAGKKHMVCKLNKSLYGLKQAPRQWYMKFDSFMKSQTYLKTYSDPCVYFKRFSENNFIILLLYVDDMLIVGKDKGLIAKLKGDLSKSFDMKDLGPAQQILGMKIVRERTSRKLWLSQEKYIERVLERFNMKNAKPVSTPLAGHLKLSKKMCPTTVEEKGNMAKVPYSSAVGSLMYAMVCTRPDIAHAVGVVSRFLENPGKEHWEAVKWILRYLRGTTGDCLCFGGSDPILSGGSKRTADGSEFEPKKKRKV

SEQ ID NO: 105 - protein, 2056 aa, Artificial Sequence
  nCas9-TnT1(D469N)RT
MAPKKKRKVGIHGVPAAMDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSAADMSEKVKNGIIPNFVTIPSTSNNPTSAESTTDEVSEQGEQPGEVIEQGEQLDEGVEEVEHPTQGEEQHQPLRRSERPRVESRRYPSTEYVLISDDREPESLKEVLSHPEKNQLMKAMQEEMESLQKNGTYKLVELPKGKRPLKCKWVFKLKKDGDCKLVRYKARLVVKGFEQKKGIDFDEIFSPVVKMTSIRTILSLAASLDLEVEQLDVKTAFLHGDLEEEIYMEQPEGFEVAGKKHMVCKLNKSLYGLKQAPRQWYMKFDSFMKSQTYLKTYSDPCVYFKRFSENNFIILLLYVDDMLIVGKDKGLIAKLKGDLSKSFDMKDLGPAQQILGMKIVRERTSRKLWLSQEKYIERVLERFNMKNAKPVSTPLAGHLKLSKKMCPTTVEEKGNMAKVPYSSAVGSLMYAMVCTRPDIAHAVGVVSRFLENPGKEHWEAVKWILRYLRGTTGDCLCFGGSDPILKGYTNADMAGDIDNRKSSTGYLFTFSGGAISWQSKLQKCVALSTTEAEYIAATETGKEMIWLKRFLQELGLHQKEYVVYCDSQSAIDLSKNSMYHARTKHIDVRYHWIREMVDDESLKVLKISTNENPADMLTKVVPRNKFELCKELVGMHSNSGGSKRTADGSEFEPKKKRKV

SEQ ID NO: 106 - protein, 617 aa, Nicotiana tabacum
AADMSEKVKNGIIPNFVTIPSTSNNPTSAESTTDEVSEQGEQPGEVIEQGEQLDEGVEEVEHPTQGEEQHQPLRRSERPRVESRRYPSTEYVLISDDREPESLKEVLSHPEKNQLMKAMQEEMESLQKNGTYKLVELPKGKRPLKCKWVFKLKKDGDCKLVRYKARLVVKGFEQKKGIDFDEIFSPVVKMTSIRTILSLAASLDLEVEQLDVKTAFLHGDLEEEIYMEQPEGFEVAGKKHMVCKLNKSLYGLKQAPRQWYMKFDSFMKSQTYLKTYSDPCVYFKRFSENNFIILLLYVDDMLIVGKDKGLIAKLKGDLSKSFDMKDLGPAQQILGMKIVRERTSRKLWLSQEKYIERVLERFNMKNAKPVSTPLAGHLKLSKKMCPTTVEEKGNMAKVPYSSAVGSLMYAMVCTRPDIAHAVGVVSRFLENPGKEHWEAVKWILRYLRGTTGDCLCFGGSDPILKGYTGADMAGDIDNRKSSTGYLFTFSGGAISWQSKLQKCVALSTTEAQYIAATETGKEMIWLKRFLQELGLHQKEYVVYCNSQSAIDLSKNSMYHARTKHIDVRYHWIREMVDDESLKVLKISTNENPADMLTKVVPRNKFELCKELVGMHSN

SEQ ID NO: 107 - protein, 2056 aa, Artificial Sequence
  nCas9-TnT1(D469G,E512Q,D545N)RT
MAPKKKRKVGIHGVPAAMDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSAADMSEKVKNGIIPNFVTIPSTSNNPTSAESTTDEVSEQGEQPGEVIEQGEQLDEGVEEVEHPTQGEEQHQPLRRSERPRVESRRYPSTEYVLISDDREPESLKEVLSHPEKNQLMKAMQEEMESLQKNGTYKLVELPKGKRPLKCKWVFKLKKDGDCKLVRYKARLVVKGFEQKKGIDFDEIFSPVVKMTSIRTILSLAASLDLEVEQLDVKTAFLHGDLEEEIYMEQPEGFEVAGKKHMVCKLNKSLYGLKQAPRQWYMKFDSFMKSQTYLKTYSDPCVYFKRFSENNFIILLLYVDDMLIVGKDKGLIAKLKGDLSKSFDMKDLGPAQQILGMKIVRERTSRKLWLSQEKYIERVLERFNMKNAKPVSTPLAGHLKLSKKMCPTTVEEKGNMAKVPYSSAVGSLMYAMVCTRPDIAHAVGVVSRFLENPGKEHWEAVKWILRYLRGTTGDCLCFGGSDPILKGYTGADMAGDIDNRKSSTGYLFTFSGGAISWQSKLQKCVALSTTEAQYIAATETGKEMIWLKRFLQELGLHQKEYVVYCNSQSAIDLSKNSMYHARTKHIDVRYHWIREMVDDESLKVLKISTNENPADMLTKVVPRNKFELCKELVGMHSNSGGSKRTADGSEFEPKKKRKV

SEQ ID NO: 108 - DNA, 6171 bp, Artificial Sequence
  nCas9-TnT1(D469G,E512Q,D545N)RT
atggctcctaagaagaagcgcaaggttgggatccacggggttccggcggctatggacaagaagtacagcatcgggctcgacatcgggacgaacagcgttgggtgggcggtcatcacggacgagtacaaggttccctccaagaagttcaaggtgctggggaacaccgaccgccactccatcaagaagaacctcatcggggccctcctgttcgacagcggggagaccgctgaggctacgaggctcaagagaaccgcgaggcgcagatacacgagaaggaagaacaggatctgctacctccaagagatcttctccaacgagatggccaaggttgacgattcattcttccaccgcctggaggagtctttcctcgtggaggaggacaagaagcacgagcggcatcccatcttcgggaacatcgtcgacgaggttgcctaccacgagaagtaccctacgatctaccatctgcggaagaagctcgtggacagcaccgataaggcggacctcagactgatctacctcgccctggcgcacatgatcaagttccgcgggcatttcctgatcgagggggatctcaacccagacaactccgatgttgacaagctgttcatccaactcgtgcagacctacaaccaactcttcgaggagaacccgatcaacgcttcgggggtcgacgctaaggccatcctgtcagcccgcctctcgaagtcacgcaggctggagaacctgatcgcgcagctcccaggggagaagaagaacgggctgttcgggaacctcatcgctctctctctggggctcacgccgaacttcaagtccaacttcgatctcgccgaggacgcgaagctgcaactcagcaaggacacctacgacgatgacctcgataacctcctggcgcagatcggggatcaatacgctgacctgttcctcgccgcgaagaacctgtcggacgccatcctcctgtcagatatcctccgcgtgaacaccgagatcacgaaggccccactctctgcgtccatgatcaagcgctacgacgagcaccatcaggatctgaccctcctgaaggcgctggtccgccaacagctcccggagaagtacaaggagatcttcttcgatcagtcgaagaacgggtacgctgggtacatcgacgggggggcgtcacaagaggagttctacaagttcatcaagccaatcctggagaagatggacgggacggaggagctcctggtgaagctcaacagggaggacctcctgcggaagcagagaaccttcgataacgggtccatcccccaccaaatccatctcggggagctgcacgccatcctgagaaggcaagaggacttctaccctttcctcaaggataaccgggagaagatcgagaagatcctgaccttcagaatcccatactacgtcgggccgctcgctcgggggaacagcagattcgcctggatgacccgcaagtcggaggagaccatcacgccgtggaacttcgaggaggtggtcgacaagggggcgagcgctcagtcgttcatcgagaggatgaccaacttcgacaagaacctgcccaacgagaaggtgctccctaagcactcgctcctgtacgagtacttcaccgtctacaacgagctcacgaaggtgaagtatgtgaccgaggggatgagaaagcctgccttcctgtccggggagcagaagaaggcgatcgtggacctcctgttcaagaccaaccggaaggtcacggttaagcaactcaaggaggactacttcaagaagatcgagtgcttcgattcggtggagatctcaggggtcgaggacaggttcaacgcctccctcgggacctaccacgatctcctgaagatcatcaaggataaggacttcctggacaacgaggagaacgaggacatcctggaggacatcgtgctgaccctcacgctgttcgaggacagggagatgatcgaggagcgcctgaagacgtacgcccatctcttcgatgacaaggtcatgaagcaactcaagagaagaagatacaccgggtgggggaggctgtcccgcaagctcatcaacgggatccgggacaagcagtcagggaagaccatcctcgacttcctgaagtctgatgggttcgccaacaggaacttcatgcaactgatccacgatgactcactcaccttcaaggaggatatccaaaaggctcaggtgagcgggcagggggactcgctgcacgagcatatcgctaacctcgctgggtcccctgcgatcaagaaggggatcctgcagaccgtgaaggttgtggacgagctcgtgaaggtcatggggcggcataagcctgagaacatcgtcatcgagatggccagagagaaccaaaccacgcagaaggggcaaaagaactctagggagcgcatgaagcgcatcgaggaggggatcaaggagctggggtcccaaatcctcaaggagcacccagttgagaacacccaactgcagaacgagaagctctacctgtactacctccagaacgggagggatatgtatgtggaccaagagctggatatcaaccgcctcagcgattacgacgttgatgctatcgtgccccagtctttcctgaaggatgactccatcgacaacaaggtcctcaccaggtcggacaagaaccgcgggaagtcagataacgttccatctgaggaggtcgttaagaagatgaagaactactggaggcagctcctgaacgccaagctgatcacgcaaaggaagttcgacaacctcaccaaggctgagagaggggggctctcagagctggacaaggctgggttcatcaagcggcagctggtcgagaccagacaaatcacgaagcacgttgcgcaaatcctcgactctcggatgaacacgaagtacgatgagaacgacaagctgatcagggaggtgaaggtcatcaccctgaagtctaagctcgtctccgacttccgcaaggacttccagttctacaaggttcgcgagatcaacaactaccaccatgcgcatgacgcttacctcaacgctgtggtcgggaccgccctgatcaagaagtacccgaagctggagagcgagttcgtgtacggggactacaaggtttacgatgtgcgcaagatgatcgccaagtcggagcaagagatcgggaaggccaccgccaagtacttcttctactcaaacatcatgaacttctttaagaccgagatcacgctggccaacggggagatccggaagagacccctcatcgagaccaacggggagacgggggagatcgtgtgggacaaggggagggatttcgcgaccgtccgcaaggttctctccatgccccaggtgaacatcgtcaagaagaccgaggtccaaacgggggggttctcaaaggagtctatcctgcctaagcggaacagcgacaagctcatcgccagaaagaaggactgggatccaaagaagtacggggggttcgacagccctaccgtcgcgtactcggttctggttgtggctaaggttgagaaggggaagtccaagaagctcaagagcgtgaaggagctcctggggatcaccatcatggagcgctccagcttcgagaagaacccaatcgacttcctggaggccaaggggtacaaggaggtgaagaaggacctgatcatcaagctcccgaagtactctctcttcgagctggagaacgggaggaagcgcatgctggcttccgctggggagctccaaaaggggaacgagctcgccctgccatccaagtatgtgaacttcctctacctggcgtcccactacgagaagctcaaggggagcccggaggacaacgagcaaaagcagctgttcgtcgagcagcacaagcattacctcgacgagatcatcgagcaaatctccgagttctccaaacgcgtgatcctcgcggacgctaacctggataaggtcctctccgcttacaacaagcaccgggacaagcccatcagagagcaggccgagaacatcatccatctcttcaccctgacgaacctcggggcccctgctgccttcaagtacttcgacaccacgatcgatcggaagagatacaccagcacgaaggaggtcctggacgcgaccctcatccaccaatctatcacggggctgtacgagacgagaatcgacctgtcacaactggggggggactccggcggctccagcggcggctccagcggcagcgagacgccgggcacgtccgagagcgccacccccgagagcagcggcggctccagcggcggctccagcgccgcggacatgtccgagaaggtgaagaacggcatcatcccgaacttcgtcaccatcccctccacgagcaacaaccccacctcggccgagtcgacgaccgacgaggtgagcgagcagggcgagcagccaggcgaggtcatcgagcagggcgagcagctggacgagggcgtggaggaggtggagcacccgacccagggcgaggagcagcaccagccactgaggaggtcggagcgcccgagggtggagtccaggaggtaccccagcaccgagtatgtgctcatctcggacgacagggagccggagtccctgaaggaggtgctcagccacccggagaagaaccagctcatgaaggcgatgcaggaggagatggagtccctgcagaagaacggcacctacaagctggtcgagctcccgaagggcaagaggcccctcaagtgcaagtgggtgttcaagctgaagaaggacggcgactgcaagctcgtccgctacaaggccaggctggtggtcaagggcttcgagcagaagaagggcatcgacttcgacgagatcttctcccccgtggtcaagatgaccagcatcaggaccatcctgtcgctggcggccagcctggacctcgaggtggagcagctggacgtcaagaccgccttcctgcacggcgacctcgaggaggagatctacatggagcagcccgagggcttcgaggtggccggcaagaagcacatggtctgcaagctgaacaagtcgctctacggcctcaagcaggccccgaggcagtggtacatgaagttcgactccttcatgaagtcccagacctacctcaagacgtactccgacccgtgcgtgtacttcaagaggttcagcgagaacaacttcatcatcctcctgctctacgtggacgacatgctcatcgtcggcaaggacaagggcctgatcgcgaagctcaagggcgacctgtccaagagcttcgacatgaaggacctcggcccggctcagcagatcctgggcatgaagatcgtgcgcgagaggacctccaggaagctgtggctcagccaggagaagtacatcgagcgcgtgctcgagaggttcaacatgaagaacgccaagccggtgtccacgcccctggctggccacctgaagctcagcaagaagatgtgccccaccacggtcgaggagaagggcaacatggctaaggtgccctactcctcggccgtgggctccctgatgtacgctatggtgtgcacccgccccgacatcgctcacgccgtgggcgtggtcagcaggttcctcgagaacccgggcaaggagcactgggaggcggtgaagtggatcctcaggtacctccggggcaccaccggcgactgcctctgcttcggtggctccgacccgatcctgaagggctacaccggcgcggacatggccggcgacatcgacaaccgcaagtccagcaccggctacctgttcacgttctccggcggcgccatctcctggcagagcaagctccagaagtgcgtggctctgagcacgaccgaggcccagtacatcgcggccacggagacgggcaaggagatgatctggctcaagcgcttcctgcaggagctgggcctccaccagaaggagtatgtggtctactgcaactcccagagcgcgatcgacctgtccaagaacagcatgtaccacgccaggaccaagcacatcgacgtgcgctaccactggatcagggagatggtggacgacgagtccctgaaggtcctcaagatcagcaccaacgagaaccccgccgacatgctcacgaaggtggtcccgaggaacaagttcgagctctgcaaggagctggtcggcatgcacagcaactccggcggcagcaagcgcacggcggacggctcggagttcgagccgaagaagaagcgcaaggtgtga

SEQ ID NO: 109 - protein, 2125 aa, Artificial Sequence
  nCas9-SH3-Tnt1-RT
MAPKKKRKVGIHGVPAAMDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGPRADPKKKRKVGGGPGAEYVRALFDFNGNDEEDLPFKKGDILRIRDKPEEQWWNAEDSEGKRGMILVPYVEKYSGDYKDHDGDYKDHDIDYKDDDDKSGAADMSEKVKNGIIPNFVTIPSTSNNPTSAESTTDEVSEQGEQPGEVIEQGEQLDEGVEEVEHPTQGEEQHQPLRRSERPRVESRRYPSTEYVLISDDREPESLKEVLSHPEKNQLMKAMQEEMESLQKNGTYKLVELPKGKRPLKCKWVFKLKKDGDCKLVRYKARLVVKGFEQKKGIDFDEIFSPVVKMTSIRTILSLAASLDLEVEQLDVKTAFLHGDLEEEIYMEQPEGFEVAGKKHMVCKLNKSLYGLKQAPRQWYMKFDSFMKSQTYLKTYSDPCVYFKRFSENNFIILLLYVDDMLIVGKDKGLIAKLKGDLSKSFDMKDLGPAQQILGMKIVRERTSRKLWLSQEKYIERVLERFNMKNAKPVSTPLAGHLKLSKKMCPTTVEEKGNMAKVPYSSAVGSLMYAMVCTRPDIAHAVGVVSRFLENPGKEHWEAVKWILRYLRGTTGDCLCFGGSDPILKGYTDADMAGDIDNRKSSTGYLFTFSGGAISWQSKLQKCVALSTTEAEYIAATETGKEMIWLKRFLQELGLHQKEYVVYCDSQSAIDLSKNSMYHARTKHIDVRYHWIREMVDDESLKVLKISTNENPADMLTKVVPRNKFELCKELVGMHSNSGGSKRTADGSEFEPKKKRKV

SEQ ID NO: 110 - DNA, 6378 bp, Artificial Sequence
  nCas9-SH3-Tnt1-RT
atggctcctaagaagaagcgcaaggttgggatccacggggttccggcggctatggacaagaagtacagcatcgggctcgacatcgggacgaacagcgttgggtgggcggtcatcacggacgagtacaaggttccctccaagaagttcaaggtgctggggaacaccgaccgccactccatcaagaagaacctcatcggggccctcctgttcgacagcggggagaccgctgaggctacgaggctcaagagaaccgcgaggcgcagatacacgagaaggaagaacaggatctgctacctccaagagatcttctccaacgagatggccaaggttgacgattcattcttccaccgcctggaggagtctttcctcgtggaggaggacaagaagcacgagcggcatcccatcttcgggaacatcgtcgacgaggttgcctaccacgagaagtaccctacgatctaccatctgcggaagaagctcgtggacagcaccgataaggcggacctcagactgatctacctcgccctggcgcacatgatcaagttccgcgggcatttcctgatcgagggggatctcaacccagacaactccgatgttgacaagctgttcatccaactcgtgcagacctacaaccaactcttcgaggagaacccgatcaacgcttcgggggtcgacgctaaggccatcctgtcagcccgcctctcgaagtcacgcaggctggagaacctgatcgcgcagctcccaggggagaagaagaacgggctgttcgggaacctcatcgctctctctctggggctcacgccgaacttcaagtccaacttcgatctcgccgaggacgcgaagctgcaactcagcaaggacacctacgacgatgacctcgataacctcctggcgcagatcggggatcaatacgctgacctgttcctcgccgcgaagaacctgtcggacgccatcctcctgtcagatatcctccgcgtgaacaccgagatcacgaaggccccactctctgcgtccatgatcaagcgctacgacgagcaccatcaggatctgaccctcctgaaggcgctggtccgccaacagctcccggagaagtacaaggagatcttcttcgatcagtcgaagaacgggtacgctgggtacatcgacgggggggcgtcacaagaggagttctacaagttcatcaagccaatcctggagaagatggacgggacggaggagctcctggtgaagctcaacagggaggacctcctgcggaagcagagaaccttcgataacgggtccatcccccaccaaatccatctcggggagctgcacgccatcctgagaaggcaagaggacttctaccctttcctcaaggataaccgggagaagatcgagaagatcctgaccttcagaatcccatactacgtcgggccgctcgctcgggggaacagcagattcgcctggatgacccgcaagtcggaggagaccatcacgccgtggaacttcgaggaggtggtcgacaagggggcgagcgctcagtcgttcatcgagaggatgaccaacttcgacaagaacctgcccaacgagaaggtgctccctaagcactcgctcctgtacgagtacttcaccgtctacaacgagctcacgaaggtgaagtatgtgaccgaggggatgagaaagcctgccttcctgtccggggagcagaagaaggcgatcgtggacctcctgttcaagaccaaccggaaggtcacggttaagcaactcaaggaggactacttcaagaagatcgagtgcttcgattcggtggagatctcaggggtcgaggacaggttcaacgcctccctcgggacctaccacgatctcctgaagatcatcaaggataaggacttcctggacaacgaggagaacgaggacatcctggaggacatcgtgctgaccctcacgctgttcgaggacagggagatgatcgaggagcgcctgaagacgtacgcccatctcttcgatgacaaggtcatgaagcaactcaagagaagaagatacaccgggtgggggaggctgtcccgcaagctcatcaacgggatccgggacaagcagtcagggaagaccatcctcgacttcctgaagtctgatgggttcgccaacaggaacttcatgcaactgatccacgatgactcactcaccttcaaggaggatatccaaaaggctcaggtgagcgggcagggggactcgctgcacgagcatatcgctaacctcgctgggtcccctgcgatcaagaaggggatcctgcagaccgtgaaggttgtggacgagctcgtgaaggtcatggggcggcataagcctgagaacatcgtcatcgagatggccagagagaaccaaaccacgcagaaggggcaaaagaactctagggagcgcatgaagcgcatcgaggaggggatcaaggagctggggtcccaaatcctcaaggagcacccagttgagaacacccaactgcagaacgagaagctctacctgtactacctccagaacgggagggatatgtatgtggaccaagagctggatatcaaccgcctcagcgattacgacgttgatgctatcgtgccccagtctttcctgaaggatgactccatcgacaacaaggtcctcaccaggtcggacaagaaccgcgggaagtcagataacgttccatctgaggaggtcgttaagaagatgaagaactactggaggcagctcctgaacgccaagctgatcacgcaaaggaagttcgacaacctcaccaaggctgagagaggggggctctcagagctggacaaggctgggttcatcaagcggcagctggtcgagaccagacaaatcacgaagcacgttgcgcaaatcctcgactctcggatgaacacgaagtacgatgagaacgacaagctgatcagggaggtgaaggtcatcaccctgaagtctaagctcgtctccgacttccgcaaggacttccagttctacaaggttcgcgagatcaacaactaccaccatgcgcatgacgcttacctcaacgctgtggtcgggaccgccctgatcaagaagtacccgaagctggagagcgagttcgtgtacggggactacaaggtttacgatgtgcgcaagatgatcgccaagtcggagcaagagatcgggaaggccaccgccaagtacttcttctactcaaacatcatgaacttctttaagaccgagatcacgctggccaacggggagatccggaagagacccctcatcgagaccaacggggagacgggggagatcgtgtgggacaaggggagggatttcgcgaccgtccgcaaggttctctccatgccccaggtgaacatcgtcaagaagaccgaggtccaaacgggggggttctcaaaggagtctatcctgcctaagcggaacagcgacaagctcatcgccagaaagaaggactgggatccaaagaagtacggggggttcgacagccctaccgtcgcgtactcggttctggttgtggctaaggttgagaaggggaagtccaagaagctcaagagcgtgaaggagctcctggggatcaccatcatggagcgctccagcttcgagaagaacccaatcgacttcctggaggccaaggggtacaaggaggtgaagaaggacctgatcatcaagctcccgaagtactctctcttcgagctggagaacgggaggaagcgcatgctggcttccgctggggagctccaaaaggggaacgagctcgccctgccatccaagtatgtgaacttcctctacctggcgtcccactacgagaagctcaaggggagcccggaggacaacgagcaaaagcagctgttcgtcgagcagcacaagcattacctcgacgagatcatcgagcaaatctccgagttctccaaacgcgtgatcctcgcggacgctaacctggataaggtcctctccgcttacaacaagcaccgggacaagcccatcagagagcaggccgagaacatcatccatctcttcaccctgacgaacctcggggcccctgctgccttcaagtacttcgacaccacgatcgatcggaagagatacaccagcacgaaggaggtcctggacgcgaccctcatccaccaatctatcacggggctgtacgagacgagaatcgacctgtcacaactggggggggacggtggcggccccagggccgacccgaagaagaagcgcaaagtgggcggcggccccggcgctgagtatgtgcgcgcgctgttcgacttcaacggcaacgacgaggaggacctgccgttcaagaagggcgacatcctccgcatcagggacaagcccgaggagcagtggtggaacgccgaggacagcgagggcaagaggggcatgatcctcgtgccctacgtcgagaagtactccggcgactacaaggaccacgatggcgactacaaggatcacgacatcgactacaaggacgacgacgacaagtccggcgccgcggacatgtccgagaaggtgaagaacggcatcatcccgaacttcgtcaccatcccctccacgagcaacaaccccacctcggccgagtcgacgaccgacgaggtgagcgagcagggcgagcagccaggcgaggtcatcgagcagggcgagcagctggacgagggcgtggaggaggtggagcacccgacccagggcgaggagcagcaccagccactgaggaggtcggagcgcccgagggtggagtccaggaggtaccccagcaccgagtatgtgctcatctcggacgacagggagccggagtccctgaaggaggtgctcagccacccggagaagaaccagctcatgaaggcgatgcaggaggagatggagtccctgcagaagaacggcacctacaagctggtcgagctcccgaagggcaagaggcccctcaagtgcaagtgggtgttcaagctgaagaaggacggcgactgcaagctcgtccgctacaaggccaggctggtggtcaagggcttcgagcagaagaagggcatcgacttcgacgagatcttctcccccgtggtcaagatgaccagcatcaggaccatcctgtcgctggcggccagcctggacctcgaggtggagcagctggacgtcaagaccgccttcctgcacggcgacctcgaggaggagatctacatggagcagcccgagggcttcgaggtggccggcaagaagcacatggtctgcaagctgaacaagtcgctctacggcctcaagcaggccccgaggcagtggtacatgaagttcgactccttcatgaagtcccagacctacctcaagacgtactccgacccgtgcgtgtacttcaagaggttcagcgagaacaacttcatcatcctcctgctctacgtggacgacatgctcatcgtcggcaaggacaagggcctgatcgcgaagctcaagggcgacctgtccaagagcttcgacatgaaggacctcggcccggctcagcagatcctgggcatgaagatcgtgcgcgagaggacctccaggaagctgtggctcagccaggagaagtacatcgagcgcgtgctcgagaggttcaacatgaagaacgccaagccggtgtccacgcccctggctggccacctgaagctcagcaagaagatgtgccccaccacggtcgaggagaagggcaacatggctaaggtgccctactcctcggccgtgggctccctgatgtacgctatggtgtgcacccgccccgacatcgctcacgccgtgggcgtggtcagcaggttcctcgagaacccgggcaaggagcactgggaggcggtgaagtggatcctcaggtacctccggggcaccaccggcgactgcctctgcttcggtggctccgacccgatcctgaagggctacaccgacgcggacatggccggcgacatcgacaaccgcaagtccagcaccggctacctgttcacgttctccggcggcgccatctcctggcagagcaagctccagaagtgcgtggctctgagcacgaccgaggccgagtacatcgcggccacggagacgggcaaggagatgatctggctcaagcgcttcctgcaggagctgggcctccaccagaaggagtatgtggtctactgcgactcccagagcgcgatcgacctgtccaagaacagcatgtaccacgccaggaccaagcacatcgacgtgcgctaccactggatcagggagatggtggacgacgagtccctgaaggtcctcaagatcagcaccaacgagaaccccgccgacatgctcacgaaggtggtcccgaggaacaagttcgagctctgcaaggagctggtcggcatgcacagcaactccggcggcagcaagcgcacggcggacggctcggagttcgagccgaagaagaagcgcaaggtgtga

SEQ ID NO: 111 - DNA, 140 bp, Artificial
  Fragment of SEQ ID NO 14 in Fig. 2
accaacctagtctccgcgctcgccgacgcgctgctcgattccgtccccatggtcgccatcacgggacaggtgtcgcgacgcatgattggcaccgacgccttccaggagacgcccatcgtcgaggtcacccgctccatcac

SEQ ID NO: 112 - DNA, 140 bp, Artificial
  Fragment of SEQ ID NO 14 in Fig. 3
caaagaagaacgaagtccgcgcagcgataaagaagatgctcgagactccagggccgtacctcttggatataatcgtcccacaccaggagcatgtgttgcctatgatccctaatggtggggctttcaaggatatgatcctg

SEQ ID NO: 113 - DNA, 210 bp, Artificial
  Fragment of SEQ ID NO 30, 31 and 32 in Fig. 4
tgggtgttatagctgtggagacacagactatgatgcagctcatccctgctgatccaggccagcttgattcccatgagcggtctgttcctcgtgctgggcaagtctggtttccagattcagttactaagacagctcaagcaatgctggacttcaaccgtgaaggattacctctgttcatccttgctaactggagaggcttctctggtgggc

SEQ ID NO: 114 - DNA, 210 bp, Artificial
  Fragment of SEQ ID NO 91 in Fig. 5
acggccacaagttcagcgtgtccggcgagggcgagggcgatgccacctacggcaagctgaccctgaagttcatctgcaccaccggcaagctgcccgtgccctggcccacccttttgaccaccttcacctacggcgtgcagtgcttcggccgctaccccgaccacatgaagcagcacgacttcttcaagtccgccatgcccgaaggctacg

SEQ ID NO: 115 - DNA, 60 bp, Artificial
  Mutated fragment of SEQ ID NO 62 in Fig. 6
tccgtgttcacatgtcttaggccctgtgattagctaagagtgtagtctagagtatggtac

SEQ ID NO: 116 - DNA, 64 bp, Artificial
  Mutated fragment of SEQ ID NO 62 in Fig. 6
gtgtggacaggaagtgaagatgtcggcctcataccaaggatggcgaccctcggatccagtatat

SEQ ID NO: 117 - DNA, 60 bp, Artificial
  Mutated fragment of SEQ ID NO 62 in Fig. 6
tccgtgttcacatgtcttaggccctgtgatttgagaagagtatagtctagagtatggtac

SEQ ID NO: 118 - DNA, 60 bp, Artificial
  Mutated fragment of SEQ ID NO 62 in Fig. 6
gtagttattgttgatgacttaattgccacctgatgaactcttggagctgccatcaaattg

SEQ ID NO: 119 - protein, 195 aa, Artificial
  Consensus sequence in Fig. 9
  misc_feature (3)..(3): Xaa can be any naturally occurring amino acid
  misc_feature (5)..(6): Xaa can be any naturally occurring amino acid
  VARIANT (7)..(7): Xaa is Ile or Leu
  misc_feature (9)..(10): Xaa can be any naturally occurring amino acid
  misc_feature (13)..(13): Xaa can be any naturally occurring amino acid
  misc_feature (15)..(15): Xaa can be any naturally occurring amino acid
  misc_feature (18)..(19): Xaa can be any naturally occurring amino acid
  misc_feature (24)..(25): Xaa can be any naturally occurring amino acid
  misc_feature (32)..(32): Xaa can be any naturally occurring amino acid
  misc_feature (36)..(36): Xaa can be any naturally occurring amino acid
  misc_feature (40)..(41): Xaa can be any naturally occurring amino acid
  misc_feature (44)..(46): Xaa can be any naturally occurring amino acid
  misc_feature (56)..(56): Xaa can be any naturally occurring amino acid
  misc_feature (58)..(59): Xaa can be any naturally occurring amino acid
  misc_feature (61)..(61): Xaa can be any naturally occurring amino acid
  misc_feature (67)..(70): Xaa can be any naturally occurring amino acid
  misc_feature (73)..(74): Xaa can be any naturally occurring amino acid
  misc_feature (77)..(77): Xaa can be any naturally occurring amino acid
  misc_feature (80)..(80): Xaa can be any naturally occurring amino acid
  VARIANT (82)..(82): Xaa is Ile or Leu
  misc_feature (84)..(84): Xaa can be any naturally occurring amino acid
  misc_feature (88)..(88): Xaa can be any naturally occurring amino acid
  misc_feature (90)..(91): Xaa can be any naturally occurring amino acid
  misc_feature (94)..(94): Xaa can be any naturally occurring amino acid
  misc_feature (96)..(96): Xaa can be any naturally occurring amino acid
  misc_feature (98)..(98): Xaa can be any naturally occurring amino acid
  misc_feature (105)..(105): Xaa can be any naturally occurring amino acid
  misc_feature (107)..(107): Xaa can be any naturally occurring amino acid
  misc_feature (112)..(112): Xaa can be any naturally occurring amino acid
  misc_feature (114)..(114): Xaa can be any naturally occurring amino acid
  misc_feature (116)..(116): Xaa can be any naturally occurring amino acid
  misc_feature (120)..(120): Xaa can be any naturally occurring amino acid
  misc_feature (122)..(122): Xaa can be any naturally occurring amino acid
  misc_feature (125)..(125): Xaa can be any naturally occurring amino acid
  misc_feature (129)..(131): Xaa can be any naturally occurring amino acid
  misc_feature (133)..(134): Xaa can be any naturally occurring amino acid
  misc_feature (136)..(136): Xaa can be any naturally occurring amino acid
  misc_feature (138)..(138): Xaa can be any naturally occurring amino acid
  misc_feature (140)..(140): Xaa can be any naturally occurring amino acid
  misc_feature (144)..(146): Xaa can be any naturally occurring amino acid
  misc_feature (149)..(149): Xaa can be any naturally occurring amino acid
  misc_feature (153)..(153): Xaa can be any naturally occurring amino acid
  misc_feature (155)..(155): Xaa can be any naturally occurring amino acid
  misc_feature (158)..(158): Xaa can be any naturally occurring amino acid
  misc_feature (161)..(161): Xaa can be any naturally occurring amino acid
  misc_feature (163)..(163): Xaa can be any naturally occurring amino acid
  misc_feature (165)..(165): Xaa can be any naturally occurring amino acid
  misc_feature (168)..(168): Xaa can be any naturally occurring amino acid
  misc_feature (170)..(171): Xaa can be any naturally occurring amino acid
  misc_feature (175)..(175): Xaa can be any naturally occurring amino acid
  misc_feature (178)..(179): Xaa can be any naturally occurring amino acid
  misc_feature (181)..(181): Xaa can be any naturally occurring amino acid
GTXGXXXFXXPGXDXSLXXYTDAXXAGDGDDXKSTXGYIXXLGXXXISWSSKKQKXVXXSXTEAEYXXXXNTXXELXWIXSXLXELGXLXXVPXVXYXDNVGATXLXANPVXHXRXKHIXLXYHXIRNXXXSXXLXVXHXSTHXXXADXLTKXLXRTXFQXAXRXIGXTXXPPSXGGXXRXADGSEFEPKKKRKV

SEQ ID NO: 120 - protein, 162 aa, Artificial
  Sequence from Cauliflower mosaic virus in Fig. 9
KEDTLYMQKVKKNLQGFPPLHHPLPEEKLIIETDASDDYWGGMLKAIKINEGTNTELICRYRSGSFKAAERNYHSNDKETLAVINTIKKFSIYLTPVHFLIRTDNTHFKSFVNLNYKGDSKLGRNIRWQAWLSHYSFDVEHIKGTDNHFADFLSREFNKVNS

SEQ ID NO: 121 - protein, 161 aa, Artificial
  Sequence from Moloney Dahlia Mosaic virus in Fig. 9
QSDTDYVKKIKKGLVNFPKLYLPKKEDSLIIETDASDHFWGGVLKAQTTEGNELICRYSSGTFKPAELNYHSNEKELLAVKQVITKFSIYLTPVTFTVRTDNVNLLKRFMNTKITGDSKQGRLIRWQMWLSHYTFNVNHLKGEKNVLADYLTREYQQKRQQ

SEQ ID NO: 122 - protein, 197 aa, Artificial
  Sequence from Moloney Murine Leukemia Virus in Fig. 9
AHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 123 - protein, 169 aa, Artificial
  Sequence from Nicotiana tabacum in Fig. 9
GTTGDCLCFGGSDPILKGYTDADMAGDIDNRKSSTGYLFTFSGGAISWQSKLQKCVALSTTEAEYIAATETGKEMIWLKRFLQELGLHQKEYVVYCDSQSAIDLSKDMLTKVVPRNKFELCKELVGMHSNNSMYHARTKHIDVRYHWIREMVDDESLKVLKISTNENPA

SEQ ID NO: 124 - protein, 16 aa, Artificial
  Sequence from Nicotiana tabacum in Fig. 9
GTTGDCLCFGGSDPIL

SEQ ID NO: 125 - protein, 174 aa, Artificial
  Sequence from Arabidopsis thaliana in Fig. 9
GTPNHGIFLKKGNTLSLHAYSDADWAGDKDDYVSTNGYIVYLGHHPISWSSKKQKGVVRSSTEAEYRSVANTSSEMQWICSLLTELGIRLTRPPVIYCDNVGATYLCANPVFHSRMKHIAIDYHFIRNQVQSGALRVVHVSTHDQLADTLTKPLSRTAFQNFASKIGVTRVPPS

SEQ ID NO: 126 - protein, 181 aa, Artificial
  Sequence from Arabidopsis thaliana in Fig. 9
GTPDHGIFLKKGNTLSLHAYSDADWAGDTDDYVSTNGYIVYLGHHPISWSSKKQKGVVRSSTEAEYRSVANTSSELQWICSLLTELGIQLSHPPVIYCDNVGATYLCANPVFHSRMKHIALDYHFIRNQVQSGALRVVHVSTHDQLADTLTKPLSRVAFQNFSRKIGVIKVPPSCGGVLRI

(19) United States
(12) Patent Application Publication
(10) Pub. No.: US 2023/0357784 A1
(43) Pub. Date: Nov. 9, 2023

(54) PRIME EDITING TECHNOLOGY FOR PLANT GENOME ENGINEERING

(71) Applicant: LIMAGRAIN EUROPE, Saint-Beauzire (FR)

(72) Inventors: Fabien NOGUÉ, Versailles (FR); Pierre-François PERROUD, Versailles (FR); Anouchka GUYON-DEBAST, Versailles (FR); Wyatt PAUL, Chappes (FR); Jean-Philippe PICHON, Chappes (FR)

(21) Appl. No.: 17/904,510

(22) PCT Filed: Feb. 19, 2021
(86) PCT No.: PCT/EP2021/054228

§ 371 Date: Aug. 18, 2022

(30) Foreign Application Priority Data
Feb. 21, 2020 (EP) 20305174.3

(51) Int. Cl. C12N 15/82; C12N 9/22; C12N 9/12; C12N 15/10

(52) U.S. Cl. CPC C12N 15/8213; C12N 9/22; C12N 9/1276; C12N 15/102; C12N 2310/20

(57) ABSTRACT

The invention relates to a method for inserting a desired edit at a target site in a double-stranded DNA sequence in a plant cell, using a Cas nickase associated with a reverse transcriptase adapted to plants and a prime-editing guide RNA comprising in particular a guide RNA region to provide targeting, a template RNA containing the desired edit and a primer binding site (PBS) for the reverse transcriptase.

Prime editing is a precise genome editing tool that directly writes new genetic information into a specified target DNA site using a catalytically impaired Cas9 endonuclease, usually a nickase Cas9, fused to an engineered reverse transcriptase, programmed with a prime editing guide RNA (pegRNA) that both specifies the target site and encodes the desired edit.

Prime Editing technology was originally described in animal cells by Liu (D. Liu. CRISPR Meeting CSHL 2019) and Anzalone et al. 2019. They used an M-MLV Reverse Transcriptase (RT) fused with a CRISPR Cas9 nickase to obtain editing in their target cells. It appeared to the inventors that the described RT was poorly adapted to plants because M-MLV RT as well as other animal virus RT (e.g. AMV) has an optimal activity above 37° C., which is a temperature that is above the adequate temperature for plant cells.

The present invention is based on the identification of reverse transcriptases that are optimally active around 25° C. and their use for performing prime editing in plants, as this temperature is well adapted for plant cells.

In this context, the inventors identified reverse transcriptases well adapted to plants and active at low temperature (˜25° C.). Plant retroviruses and retrotransposons are sources of reverse transcriptases. Plants have many retrotransposons classes, the LTR retrotransposons falling into 2 superfamilies the Ty1/Copia family and the Ty3/Gypsy family (Neumann et al. 2019). The plant retroviruses include the pararetroviruses; dsDNA viruses that replicate by reverse transcription of an RNA intermediate. The pararetrovirus cauliflower mosaic virus (CaMV) genome carries a reverse transcriptase (78 KDa) domain coded by its ORF V (Takatsuji et al. 1986 and 1992). The plant viral or retrotransposon -RT efficiency at low temperature is well adapted to plant physiology including in vitro.

Using reverse transcriptases adapted to plants and a nickase (protein able to introduce a cut in a single DNA strand), the present invention allows the introduction of one or several mutations (the desired edit) at a target site in a plant genome in a single experiment without relying on homologous recombination.

Introduction of such mutations is called gene editing as it can be performed to modify (edit) a specific or several specific base(s) of a given gene of the plant genome. The introduction of the desired edit can be performed to modify a regulatory sequence a given gene of the plant genome.

### SUMMARY OF THE INVENTION

In a first aspect, the invention relates to a method for inserting a desired edit at a target site in a double-stranded DNA sequence in a plant cell, comprising:
a. providing a Cas nickase associated with a reverse transcriptase adapted to plants and a prime-editing guide RNA (pegRNA) to the double-stranded DNA sequence, wherein the pegRNA comprises (from 5′ to 3′):
   i. a single guide RNA region which hybridizes to a DNA strand at the target site (thereby directing the Cas nickase associated with the reverse transcriptase to the site),
   ii. a guide scaffold sequence which allows Cas-binding,
   iii. a template RNA containing the desired edit, and which will serve as the template for creating the edited DNA strand, and
   iv. a primer binding site (PBS) that allows the 3′end of the nicked DNA strand to hybridize to the pegRNA, and will serve for the reverse transcriptase to start reverse transcription;
b. wherein one strand of the double-stranded DNA sequence is cut by the nickase thereby generating a free single-strand DNA (ssDNA) having a 3′ end;
c. wherein the 3′ end of the free ssDNA hybridizes to the PBS of the pegRNA,
d. wherein the reverse transcriptases performs reverse transcription of the template RNA of the pegRNA and elongates the 3′ extremity of the ssDNA,
e. thereby generating a ssDNA flap comprising the desired edit and which is complementary to the DNA synthesis template;
f. wherein the endogenous DNA strand adjacent to the cut site is replaced with the ssDNA flap, thereby installing the desired edit at the target site in the double-stranded DNA sequence.

In particular, the plant cell is present in a plant tissue or in a whole plant. In particular, the plant cell is present in solution.

In another aspect, the invention relates to a method (which can be performed in vitro) for obtaining a plant having a desired edit at a target site
a. providing to a plant cell or plant tissue comprising, in its genome, a sequence of interest containing the target site,
b. a Cas nickase associated with a reverse transcriptase adapted to plants and a prime-editing guide RNA (pegRNA), and wherein the pegRNA comprises (from 5′ to 3′):
   i. a single guide RNA region which hybridizes to a DNA strand at the target site (thereby directing the nickase and reverse transcriptase to the site),
   ii. a guide scaffold sequence which allows Cas-binding,
   iii. a template RNA containing the desired edit, and which will serve as the template for creating the edited DNA strand, and
   iv. a primer binding site (PBS) that allows the 3′end of the nicked DNA strand to hybridize to the pegRNA, and will serve for the reverse transcriptase to start reverse transcription
c. culturing the plant cell or plant tissue in adequate conditions for multiplication of cells
d. growing a plant from the cultured plant cell(s) or plant tissues.

In an embodiment, the method further comprises, after step c) and before step d), screening the cultured plant cell(s) or plant tissue(s) from step c) to identify the cells containing the desired edit introduced at the target site and isolated such cells, and wherein the plant is grown in step d) if the screen indicated that the desired edit was introduced at the target site. This leads to a method (which may be performed in vitro) for obtaining a plant having a desired edit at a target site
a. providing to a plant cell or plant tissue comprising, in its genome, a sequence of interest containing the target site,
b. a Cas nickase associated with a reverse transcriptase adapted to plants and a prime-editing guide RNA (pegRNA), and wherein the pegRNA comprises (from 5′ to 3′):
   i. a single guide RNA region which hybridizes to a DNA strand at the target site (thereby directing the nickase and reverse transcriptase to the site),
   ii. a guide scaffold sequence which allows Cas-binding,
   iii. a template RNA containing the desired edit, and which will serve as the template for creating the edited DNA strand, and
   iv. a primer binding site (PBS) that allows the 3′end of the nicked DNA strand to hybridize to the pegRNA, and will serve for the reverse transcriptase to start reverse transcription
c. culturing the plant cell or plant tissue in adequate conditions for multiplication of cells
d. screening the cultured plant cell(s) or plant tissue(s) from step c) to identify the cells containing the desired edit introduced at the target site and isolated such cells
e. growing a plant from the cultured plant cell(s) or plant tissues if the screen performed in d. indicated that the desired edit was introduced at the target site

The methods above can also present one or more of the following:
the Cas nickase is a mutated Cas9 protein, in particular the H840A Cas9 protein represented by SEQ ID NO: 39.
the reverse transcriptase is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 64, SEQ ID NO: 85, SEQ ID NO: 87 and SEQ ID NO: 106, or a modified reverse transcriptase based on these sequences.
the Cas nickase protein is fused with the reverse transcriptase adapted to plants
it further comprises the delivery of a supplementary single guide RNA (gRNA) targeting a secondary site in the vicinity (5′ or 3′ of the target site) of the target site, so as to induce a Cas nickase to introduce a nick in DNA sequence at the secondary site.
the plant is a monocotyledon, preferably a cereal.

In another aspect, the invention pertains to a vector comprising a DNA construct coding for a Cas nickase and a DNA construct coding a reverse transcriptase adapted to plants, with the genetic elements allowing transcription in a plant cell. In particular, the DNA constructs coding for the Cas nickase and the reverse transcriptase adapted to plants are fused, leading to production of a fusion protein comprising the Cas nickase fused with the reverse transcriptase adapted to plants.

In another aspect, the invention pertains to a fusion protein comprising a Cas nickase fused with a reverse transcriptase adapted to plants.

In another aspect, the invention pertains to a kit to perform the methods herein disclosed, comprising one or multiple vectors wherein the one or multiple vectors comprise(s) a sequence coding for a Cas nickase, a sequence coding for a reverse transcriptase adapted to plants, and a sequence transcribed to a pegRNA as herein defined. In particular, the kit comprises two vectors, wherein one of the vectors comprises a sequence coding for the Cas nickase and a sequence coding for the reverse transcriptase adapted to plants, preferably fused within the same gene, and the other vector contains the sequence transcribed to the pegRNA. In one embodiment, the kit comprises one vector, which contains a sequence coding for the nickase and a sequence coding for the reverse transcriptase adapted to plants, wherein these sequences are preferably fused within the same gene, as well as the sequence transcribed to the pegRNA.

The invention also relates to a complex comprising a Cas nickase associated by binding domains with a reverse transcriptase adapted to plants

The invention also discloses and relates specific modified plant-adapted reverse transcriptases, in particular depicted as SEQ ID NO: 85, SEQ ID NO: 87 or SEQ ID NO: 106.

The invention also relates to a plant comprising, in its genome or in an extrachromosomal vector, the DNA construct as described above. In an embodiment, the plant further comprises, in its genome or in an extrachromosomal vector, a pegRNA guide as defined above, wherein the single guide RNA region hybridizes to a DNA strand at a target site, and the template RNA contains a edit desired to be performed at the target site.

The invention also relates to the use of a reverse transcriptase adapted to plants associated with a Cas nickase and a prime-editing RNA comprising from 5′ to 3′ (i) a template RNA containing the desired edit, to serve as the template for creating the edited DNA strand upon reverse transcription by the reverse transcriptase, and (ii) a primer binding site (PBS) that allows the 3′end of the nicked DNA strand to hybridize to the prime-editing guide RNA, to serve for initiating reverse transcription by the reverse transcriptase, for introducing desired edit at a target site in plants. In particular, the reverse transcriptase and the Cas nickase are fused in the same polypeptide. In one embodiment, the Cas nickase is a mutated Cas protein, in particular the H840A Cas9 protein represented by SEQ ID NO: 39. In one embodiment, the reverse transcriptase is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 64, SEQ ID NO: 85, SEQ ID NO: 87 and SEQ ID NO: 106. In particular, the Cas nickase protein is fused with the reverse transcriptase adapted to plants. In particular the plant is a monocotyledon, preferably a cereal.

In some embodiments, the methods are performed in vitro.

It is preferred when the methods of the invention are performed at a temperature between 22° C. and 28° C.

### DETAILED DESCRIPTION OF THE INVENTION

In a first embodiment and in order to perform one embodiment of the methods herein disclosed, the invention relates to a genetic construct comprising a nucleic acid (gene) coding for a Cas nickase protein associated with a reverse transcriptase adapted to plants or a modified version of a reverse transcriptase adapted to plants.

A Cas nickase relates to a protein or polypeptide that have endonuclease activity and that is able to introduce a break in one single strand of double-stranded DNA and that uses the CRISPR mechanism (Clustered Regularly Interspaced Short Palindromic Repeats) to bind to DNA at a location specified by guide RNA. This system is known in the art, as well as the Cas (CRISPR associated protein) proteins associated with this mechanism. WO2014093661 or WO2013176772 (CRISPR/Cas9) and WO2016205711 (CRISPR/Cas12a) describe methods for targeting a specific location with a Cas protein, using an appropriate guide RNA.

It is preferred to use a mutated Cas protein, in particular a mutated Cas9 protein. It is reminded that the ability of Cas9 to create a double strand DNA break is mediated by two domains having nuclease activity, a RuvC domain and an HNH domain. If one of these domains is mutated, the Cas9 enzyme loses its ability to cut the double-stranded DNA and can only cut one strand (and thus becomes a nickase). Mutation D10A in Cas9 eliminates the activity of the RuvC domain and H840A eliminates the activity of the HNH domain.

Such nCas9 protein (H840A) is described, for instance as SEQ ID NO: 39. This H840A mutated Cas9 protein is preferred as it cuts the DNA strand complementary to the strand to which the guide RNA is bound. Alternatively, D10A Cas9 mutated protein could also be used in the context of the methods herein described. In another embodiment, one can use a Cas12a (Zetsche B et al., 2015), with a nickase activity. One can use the AsCas12a with the R1226 mutation described in WO2016205711. In another embodiment, one can use the Cas12b protein (Ming et al 2020 CRISPR-Cas12b enables efficient plant genome engineering).

The nickase can be associated with Nuclear Localization Signals (NLS) like the SV40 NLS (SEQ ID NO: 81) or the XlNucleoplasmin NLS (SEQ ID NO: 82). The NLS can be situated at one or both ends of the nickase protein.

The NLS ensures that the nickase is transported in the nucleus where the editing takes place.

A reverse transcriptase (RT) is an enzyme that can generate complementary DNA (cDNA) from an RNA template. Reverse transcription starts from a DNA primer annealed to the RNA strand, and the enzymes synthesize DNA from the 3′ end of the primer in the 5′ to 3′ direction (with respect to the newly synthesized DNA strand). As envisaged herein the term “reverse transcriptase adapted to plants” relates to a protein that has reverse transcriptase activity and whose reverse transcriptase efficacy at 25° C. is better than reverse transcriptases identified in animal viruses. An in vitro test can be performed to compare the efficacy at 25° C. of MMLV-RT and a reverse transcriptase. The efficacy can be measured by comparing the quantity of cDNA produced from an RNA template (from 15 to 60 bp) during a 10- or 15-minutes period. A RT is adapted to plants when the quantity of cDNA produced with the RT is increased by at least 50% compared to the quantity of cDNA produced by the MMLV-RT in these conditions. Examples of such reverse transcriptases are provided above. These can also be modified as long as they retain reverse transcriptase activity and are adapted to plants. Modifications can be designed to improve the reverse transcriptase activity. The modification can be targeted in the RNase H domain. The modification can be a complete deletion of the RNase H domain or one or several point mutations. Known mutations in MMLV-RT (SEQ ID NO: 9) that reduce RNaseH activity include mutations at positions D524, S526, D583, Y586 and D653. It is possible to create those same mutations in reverse transcriptases adapted to plants. (FIG. 9)

The nickase is associated with a reverse transcriptase adapted to plants. This indicates that the reverse transcriptase and the nickase are physically and spatially close to each other, so that reverse transcription can start quickly when the DNA strand has been cut by the nickase and the primer has bound to the cut strand.

The reverse transcriptase adapted to plants can be associated with the nickase protein by various ways in the Cas prime editing complex.

As seen above, the two proteins can be associated in the same polypeptide (this is obtained in particular when using a nucleic acid as disclosed herein). This embodiment is preferred. The reverse transcriptase can thus be fused to a nickase. Such fusion can be a genetic fusion (the ORFs (open reading frames) of each of the proteins can be placed in frame to form a new ORF which codes for a polypeptide containing the amino acids of the two proteins (generally with spacer amino acids between them). The reverse transcriptase adapted to plants and the nickase can be associated with the reverse transcriptase adapted to plants in N-terminus and the nickase in C-terminus of the fusion or with the reverse transcriptase adapted to plants in C-terminus and the nickase in N-terminus in the fusion. The 16-residue XTEN linker, known in the art, can be used to bridge the reverse transcriptase adapted to plants and the nickase in the fusion protein.

Alternatively, the reverse transcriptase adapted to plants can be linked to the nickase protein using a chemical linker. Such linkers may comprise reactive moieties including such as aminoxy groups, azido groups, alkyne groups, thiol groups or maleimido groups, either alone or in combination. Generally, the linkers comprise two functional moieties, one providing rapid and efficient labeling and another enabling rapid and efficient coupling of the polypeptides, in particular through an amine group or preferably through the thiol group of the cysteine. Preferably, the complex is formed by first reacting one protein with the linker, and subsequently with the thiol group of the other protein.

The nickase can also be bound to the reverse transcriptase adapted to plants using binding domains, Protein-protein interaction domains, or intein. In the protein-protein interaction domains embodiment, each of the nickase and of the reverse transcriptase adapted to plants are modified so as to contain protein-protein interaction domains that are complementary to each other. When the two proteins are close to each other (which happens within the nucleus), the two domains bind to each other thereby associating the nickase and the reverse transcriptase adapted to plants.

One can cite the dockerin/cohesin system described in You et al (2012). One can also use the system involving FK506 binding protein 12 (FKBP), and FKBP rapamycin binding (FRB) domain used to create a split Cas9 in Zetsche et al. (2015).

Alternatively, the reverse transcriptase is brought to the nickase Cas via a binding to the pegRNA. In this embodiment, the pegRNA may comprise MS2 hairpins. Upon binding to the nickase Cas, the pegRNA-MS2 is able to recruit (thanks to the MS2 hairpins) reverse transcriptase fused to MS2 bacteriophage coat protein (MCP). This system is described in Hess et al. 2016.

In a preferred embodiment, the reverse transcriptase adapted to plants and the nickase protein are associated in a fusion protein. In a specific embodiment, the fusion protein is SEQ ID NO: 10, SEQ ID NO: 11, SEQ ID NO: 12, SEQ ID NO: 65, SEQ ID NO: 104, SEQ ID NO: 105, SEQ ID NO: 107, SEQ ID NO: 109.

### Providing Proteins and Guides to the Sequence of Interest

The proteins can be provided to the nucleotide sequence of interest by multiple ways. It is reminded that it is preferred when the proteins (nickase and reverse transcriptase) can reach the target site using the CRISPR system and in particular use of a guide RNA (which is further described below) and a mutated Cas nickase. Introduction of the proteins and guide RNA within the cells is to be obtained so they can act in the cell nucleus. It can be done directly as RiboNucleoprotein (RNP) (the proteins and guide RNA are pre-assembled and directly introduced within the cells) or indirectly (vectors are introduced within the cells and the proteins and guide RNA are produced inside the cells). One can also deliver the proteins, nucleic acid (DNA, mRNA) coding for the proteins or for the guide RNAs using conjugation Cell Penetrating Peptides (CPP), nanoparticles, or biolistics.

In one embodiment, the proteins and guide RNA are introduced within the cells, by the use of vectors, as transgenes, the proteins being produced by the cell machinery (after transcription and translation) and the guide RNA being transcribed by the cell machinery. Such transgenes can be introduced within the genome of the cells (genomic integration) or present on extrachromosomal vectors (such as plasmids or artificial chromosomes).

The DNA constructs used in these methods are introduced in the genome of the cells by transgenesis, through any method known in the art. In particular, it is possible to cite methods of direct transfer of genes such as direct micro-injection into embryos or nuclei, vacuum infiltration or electroporation, direct precipitation by means of PEG or the bombardment by gun of particles (preferably gold particles) covered with the DNA of interest. When the cells are plant cells, it is preferred to transform them with a bacterial strain, using in particular Agrobacterium bacterial strains, and preferably Agrobacterium tumefaciens. One can also introduce the transgenes by protoplast transformation.

The sequence encoding the proteins and the guide RNA (prime editing guide RNA or pegRNA) are under the control of adequate promoters, in particular promoters operative in plants (i.e. which drives transcription of the gene which it controls in plants). One can use, as an illustration, a constitutive promoter, a tissue-specific promoter (and in particular a promoter that is expressed in embryos, in pollen or in ovarian cells), or an inducible promoter. When working on plants, and although some promoters may have the same pattern of regulation when there are used in different species, it is often preferable to use monocotyledonous promoters in monocotyledons and dicotyledonous promoters in dicotyledonous plants.

Examples of constitutive promoters useful for expression include the 35S promoter or the 19S promoter (Kay et al., 1987), the rice actin promoter (McElroy et al., 1990), the pCRV promoter (Depigny-This et al., 1992), the CsVMV promoter (Verdaguer et al., 1998), the ubiquitin 1 promoter of maize (Christensen et al., 1996) and the ubiquitin promoter from rice or sugarcane.

Other promoters of the invention are the U3 promoter (P. patens U3 promoter SEQ ID NO: 82) and the U6 promoter (P. patens U6 promoter SEQ ID NO: 79; ZmU6 promoter (SEQ ID NO: 15), TaU6 promoter (SEQ ID NO: 33).

These genetic sequences shall also preferably contain any genetic elements (terminators, 5′UTR . . . ) making it possible to obtain or optimize the expression of the nucleic acid. Such genetic elements are known in the art and can be selected by the person of skill in the art depending on the plant in which the genetic construct shall be expressed and/or the cell type in which expression is required.

The reverse transcriptase, the nickase and the guide can be cloned in a single expression cassette in a single vector or in several cassettes in the same vector or in several cassettes in several vectors.

### Screening for Edition of the Nucleotide

It is preferred when the cells are exposed to the reverse transcriptase and the nickase that they are cultured in conditions appropriate to allow chromosome replication and mitosis (the conditions are similar to that used for classical CRISPR-Cas sequence modification).

Screening can be performed by any method known in the art, in particular as performed for other methods of CRISPR-Cas sequence modification. One can, for instance, isolate the DNA from the part of the cultured cells and sequence the sequence of interest to verify that the desired edit was inserted at the target site. This makes also possible to quantify the number of cells in which edition occurred. Alternatively, one can use probes appropriate to detect the desired edit at the target site.

By way of example, one can extract the DNA of a cell, of a tissue or an organism, amplify the nucleotide sequence of interest with specific primers by PCR and sequence the sequence of interest to detect the presence of the expected modification. The sequencing can be implemented using NEXT Generation Sequencing (NGS). One can also use the droplet digital PCR method (ddPCR™ BIO RAD) or the KASP (Biosearch Technologies) method based on detection of fluorescence. One can also use phenotypic screening, for example if the prime editing creates a mutation allowing the cell to resist to a toxic component, the screening can be made on a medium comprising such toxic component.

In another embodiment, it is possible to use a plant sample from cultured cells to screen for the presence of the desired edit at the target site. If present, the cells can be cultured in vitro and regenerated to whole plants.

In another embodiment, if the desired edit at the target site creates a mutation allowing the plant cell to resist to a toxic component (such as an herbicide), the screening can be made on a medium comprising such toxic component. The plant cell can be regenerated to a whole plant.

The invention is preferably performed on plant cells or in plant tissues, as it uses reverse transcriptase that are optimized for such cells. It could however also be performed on other types of cells such as fungal cells or animal cells.

Plant tissues can be embryos, shoot apical meristem (SAM), plant parts like pollen, microspores, leaves or plant explants.

One can perform the method on mosses like P. patens. One can perform the method on monocotyledonous plant cells. It is also possible to perform the method on dicotyledonous plant cells. Among monocotyledonous plants, one can cite cereals like rice, wheat, barley, sorghum, oat, maize but also sugarcane. Among dicotyledonous plants, one can cite soybean, cotton, tomato, beet, sunflower, or rapeseed.

When the method is performed on plant cells, one can use the totipotency property of such plant cells, which makes it possible to regenerate a whole plant from a given cell (for instance after growing the cell and forming a callus from the cultured cells).

In particular, the present invention also relates to a method to perform prime editing in plants by delivering to a plant a Cas9 nickase protein (nCas9) associated with a reverse transcriptase adapted to plants or a modified version of a reverse transcriptase adapted to plants and a prime-editing guide RNA (pegRNA).

Such Cas9 nickase protein (nCas9) associated with a reverse transcriptase adapted to plants or a modified version of a reverse transcriptase adapted to plants is preferably expressed directly in the plant, after introduction of a genetic construct as disclosed within the plant.

It is preferred if the nCas9 and the reverse transcriptase adapted to plants are fused.

The invention also relates to a plant cell or a plant containing, in its genome, a genetic construct as disclosed above.

The invention also relates to a bacterial cell containing a genetic construct as disclosed above, in its genome or in a plasmid or cosmid.

It is preferred when such method is performed in vitro.

It is preferred when such method is performed between 22° C. and 28° C.

Reverse transcriptases adapted to plants are, by way of illustration, reverse transcriptase from plant retroviruses and retrotransposons. Examples of such plant expressed reverse transcriptases are:
AtRE1 and AtRE2—from Arabidopsis retrotransposons (SEQ ID NO: 3 and SEQ ID NO: 4)
OsLullaby-Chr2—from Rice retrotransposons (SEQ ID NO: 5)
CaMV RT ORF N-termdel—Cauliflower Mosaic Virus N-terminal ORV deletion (SEQ ID NO: 1)
DVM-H—Dahlia Mosaic Virus (SEQ ID NO: 2)
OsRIRE7—from Rice RIRE7 retrotransposons (SEQ ID NO : 6)
tnt1 from Nicotiana tabacum, a mobile retroviral-like transposable element (SEQ ID NO: 7)
OsKarma_RT domain (SEQ ID NO: 64)
Tnt1_RTv2_domain—modified tntl (SEQ ID NO: 85)
Tnt1(D469N)_RT_domain—modified tnt1 in position 469 (SEQ ID NO: 87)
Tnt1(D469G,E512Q,D545N)_RT_domain—modified tnt1 in positions 469, 512 and 545 (SEQ ID NO: 106)

The invention thus relates to a genetic construct coding for a fusion protein comprising SEQ ID NO: 39 and SEQ ID NO: 1.

The invention thus relates to a genetic construct coding for a fusion protein comprising SEQ ID NO: 39 and SEQ ID NO: 2.

The invention thus relates to a genetic construct coding for a fusion protein comprising SEQ ID NO: 39 and SEQ ID NO: 3.

The invention thus relates to a genetic construct coding for a fusion protein comprising SEQ ID NO: 39 and SEQ ID NO: 4.

The invention thus relates to a genetic construct coding for a fusion protein comprising SEQ ID NO: 39 and SEQ ID NO: 5.

The invention thus relates to a genetic construct coding for a fusion protein comprising SEQ ID NO: 39 and SEQ ID NO: 6.

The invention thus relates to a genetic construct coding for a fusion protein comprising SEQ ID NO: 39 and SEQ ID NO: 7.

The invention thus relates to a genetic construct coding for a fusion protein comprising SEQ ID NO: 39 and SEQ ID NO: 64.

The invention thus relates to a genetic construct coding for a fusion protein comprising SEQ ID NO: 39 and SEQ ID NO: 85.

The invention thus relates to a genetic construct coding for a fusion protein comprising SEQ ID NO: 39 and SEQ ID NO: 87.

The invention thus relates to a genetic construct coding for a fusion protein comprising SEQ ID NO: 39 and SEQ ID NO: 106.

The coupling of the nCas9 and the reverse transcriptase is done with a linker to create a fusion protein nCas9-RT.

Preferably, the nCas9 polypeptide is placed at the N-terminus of the fusion protein. In another embodiment, the nCas9 polypeptide is located at the C-terminus of the fusion protein.

### Description of the Prime-Editing Guide RNA (pegRNA)

The prime-editing guide RNA (pegRNA) comprises (from 5′ to 3′):
(a) a single guide RNA region which hybridize to a DNA strand at the target site (thereby directing the nickase and reverse transcriptase to the site),
(b) the guide scaffold sequence which allows Cas-binding,
(c) a template RNA containing the desired edit, and which will serve as the template for creating the edited DNA strand, and
(d) a primer binding site (PBS) that allows the 3′end of the nicked DNA strand to hybridize to the pegRNA, and will serve for the reverse transcriptase to start reverse transcription.

The desired edit can be one or several nucleotide substitutions, insertions or deletions, combinations of nucleotide substitutions and indels.
The desired edit can be performed to modify a regulatory sequence, a given gene (or coding sequence) of a genome, preferably a plant genome.

The desired edit can provide a trait improvement to the plant such as tolerance to biotic or abiotic stress, improved plant architecture, improved quality, improved yield, improved tolerance to herbicide.

After nicking of the PAM strand, the newly created 3′ free extremity of a ssDNA molecule hybridizes to the PBS region of the pegRNA molecule. Then, the reverse transcriptase associated with the nickase protein is capable of initiating reverse transcription of the template RNA moiety of the pegRNA and to elongate the 3′ extremity of the ssDNA. The ssDNA flap created is then resolved to form a double-stranded edited DNA, by the cell repair machinery.

The PBS is designed so as to be adapted according to the target sequence. The length of the PBS varies between 8 and 15 nucleotides.

### Improving Integration of the Desired Edit

In order to improve integration of the desired edit when the nicked double-strand DNA is repaired, it is possible to introduce another nick in the vicinity (within 300 base pairs (bp) 5′ or 3′) of the target site. Hence, one can provide or deliver a supplementary single guide RNA (gRNA), which targets a secondary site in the vicinity of the target site. Such supplementary single guide RNA will allow the Cas nickase to introduce a nick at the site where it has bound the double-stranded DNA. In one embodiment, the supplementary single guide RNA is designed so that the Cas nickase will nick the non-edited strand.

### Transgenic Plants for Performing the Methods

The invention also relates to a plant comprising, in its genome or in an extrachromosomal vector, DNA constructs herein described. The plant may thus contain, in its genome, a DNA construct coding for a Cas nickase and a DNA construct coding a reverse transcriptase adapted to plants, with the genetic elements allowing transcription in a plant cell. These two constructs are transgenes that have been integrated in the plant's genome by any method known in the art. In another embodiment, the DNA construct codes for a Cas nickase fused to the reverse transcriptase adapted to plants, leading to production of a fusion protein comprising the Cas nickase fused with the reverse transcriptase adapted to plants. It is also foreseen that these DNA constructs are present in a vector that is extrachromosomal (not in the genome of the plant), in particular when transient expression is desired.

In an embodiment, the plant further comprises, in its genome or in an extrachromosomal vector, a pegRNA guide as herein defined, wherein the single guide RNA region hybridizes to a DNA strand at a target site, and the template RNA contains a edit desired to be performed at the target site.

The fusion protein, the pegRNAs and the supplementary gRNA can be delivered into plants as ribonucleoprotein complexes (RNP), as vectors comprising genetic constructs encoding the different elements.

The delivery methods are known by the skilled person. For example, mention may be made of electroporation, biolistics, virus-mediated transformation, Agrobacterium mediated plant transformation (Ishida et al., 1996)

The present invention relates to plants edited by the prime editing for plants method.

Plants according to the invention are monocotyledonous plants such as cereals like maize, wheat, barley, rice, sorghum, oat but also sugarcane or dicotyledonous plants such as soybean, cotton, rapeseed, sunflower, tobacco, tomato.

### FIGURES

FIG. 1: Treemap of sequences of plant-expressed reverse transcriptase (RT) domains, Drosophilia Copia RT and the MMLV RT domain present in the PE2 editor

FIG. 2: ZmALS2 region targeted for the introduction of the Pro-165-Ser mutation. The sequence shown is part of SEQ ID NO: 14, and shows the desired product with the C to T change. pegRNA 488 (spacer 488forw+488 PBS (Primer Binding Site) and 488 RT template) are used with gRNA 443rev to create a nick on the non-edited DNA strand. pegRNA 493 (spacer 493rev+493 PBS and 493 RT template) are used with gRNA 525forw to create a nick on the non-edited DNA strand.

FIG. 3: ZmALS2 region targeted for the introduction of the Ser-621-Asn mutation. The sequence shown is part of SEQ ID NO: 14, and shows the desired product with the G to A change. pegRNA 1863 (spacer 1863forw+1863 PBS and 1863 RT template) are used with gRNA 1809rev to create a nick on the non-edited DNA strand. pegRNA 1858 (spacer 1858rev+1858 PBS and 1858 RT template) are used with gRNA 1800forw to create a nick on the non-edited DNA strand.

FIG. 4: TaACCase region targeted for the introduction of the Ala-2004-Val mutation.

The sequence shown is part of SEQ ID NO: 30, 31 and 32, and shows the desired product with the C to T change. pegRNA 1275 (spacer 1275rev+1275 PBS and 1275 RT template) are used with gRNA 1178forw and/or gRNA1205forw to create a nick or nicks on the non-edited DNA strand.

FIG. 5: BFPmm region after editing and the introduction of the His-67-Tyr mutation

The sequence shown is part of SEQ ID NO: 91 (GFPmm). The figure shows the pegRNA (nCas9-PE_BE gRNA+PBS+RT—template). A to G for Cas9 PAM and CG to TT indicate the modifications in BFP to introduce PAM for Cas9 and Cas12a. nCas9_gRNA_R3 and gRNA_PE3b are the supplementary gRNAs used to create a second nick on the non-edited strand. T to C in BFP indicates the target nucleotide to revert the BFPmm to GFPmm. The codon 67 CAC encodes His in BFPmm and after the edit, the codon 67 is TAC and encodes Tyr in GFPmm.

FIG. 6: APT region before and after edit

The sequence shown is part of SEQ ID NO: 62. The figures present the position of the different elements (Primer binding site, RT template, tracrRNA) of the pegRNA (pegRNA-APT #1, pegRNA-APT #2, pegRNA-APT #3, pegRNA-APT #6). SSB stands for single-strand break. The lower-case letters indicate the mutations.

FIG. 7: Prime Editing experiments in P. patens. Each column of the table indicates a combination of plasmids (p) that are transformed into P. patens protoplasts. C=control transformation, N−=no second DNA nick (PE2). N=Second DNA nick (PE3).

FIG. 8: Prime Editing experiments in maize and wheat protoplasts. Each column of the table indicates a combination of plasmids (p) that are transformed into protoplasts. C=control transformation, PE2=no second DNA nick. PE3=Second DNA nick (PE3). PE3b=Second DNA nick on non-edited DNA strand after Prime Editing.

FIG. 9: Alignment of the RNase H domains of different reverse transcriptases and the position of the mutations to improve the activity (D524, E562, 583) The arrows indicate the position to mutate.

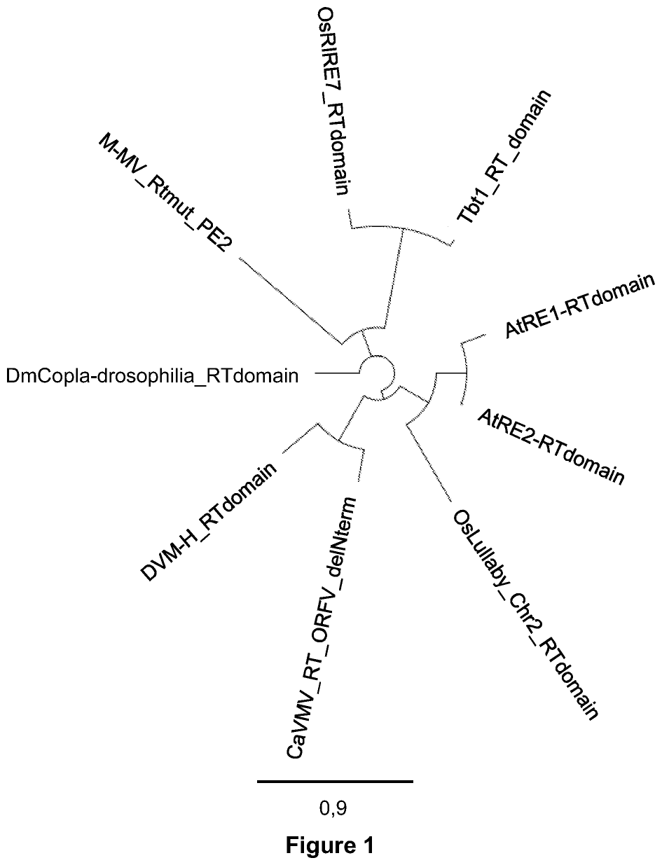

### EXAMPLES

### Example 1: Identification of Plant-Expressed Reverse Transcription Domains

Plant retroviruses and retrotransposons are potential sources of reverse transcriptases (RTs). Plants have many retrotransposons classes, the LTR retrotransposons falling into 2 superfamilies the Ty1/Copia family and the Ty3/Gypsy family (Neumann et al. 2019). The plant retroviruses include the pararetroviruses; dsDNA viruses that replicate by reverse transcription of an RNA intermediate. The pararetrovirus cauliflower mosaic virus (CaMV) genome carries a reverse transcriptase (78 KDa) domain coded by its ORF V (Takatsuji et al. 1986 and 1992). An N-terminally truncated version of ORF V was found to be functional in yeast (Takatsuji et al. 1992). By homology to this truncated ORFV other plant-expressed RT domains were identified (SEQ ID NO: 1-7) and were aligned with the Drosophila Copia RT domain (SEQ ID NO: 8) and the PE2 editor M-MLV RT domain (SEQ ID NO: 9) (Anzalone et al. 2019).

The M-MLV RT domain in the PE2 editor from Anzalone et al. is a MMLV RT pentamutant mutated at the following positions: D200N/L603W/T330P/T306K/W313F.

The PE2 editor from Anzalone et al. is a nickase Cas9 nCas9(H840A) fused to M-MLV RT pentamutant D200N/L603W/T330P/T306K/W313F.

### Example 2: Activity of Prime Editors with Plant-Expressed RT Domains in P. patens

Disruption of the P. patens adenine phosphoribosyltransferase (APT) (SEQ ID NO: 62 encoding SEQ ID NO: 63) gene function leads to resistance of P. patens protoplasts to the chemical 2-Fluoroadenine (2-FA) which is present at 10 uM in the media, since the active ATP metabolizes 2-FA to the cytotoxic 2-FluoroAMP. This 2-FA resistance has been used as a powerful screen to identify APT mutations since only loss of function in APT leads to development of plants from the protoplasts (Trouiller et al., (2006)). This positive selection screen can be used for optimizing GE tools and is used to test nCas9-MMLVRT and nCas9-plantRT versions.

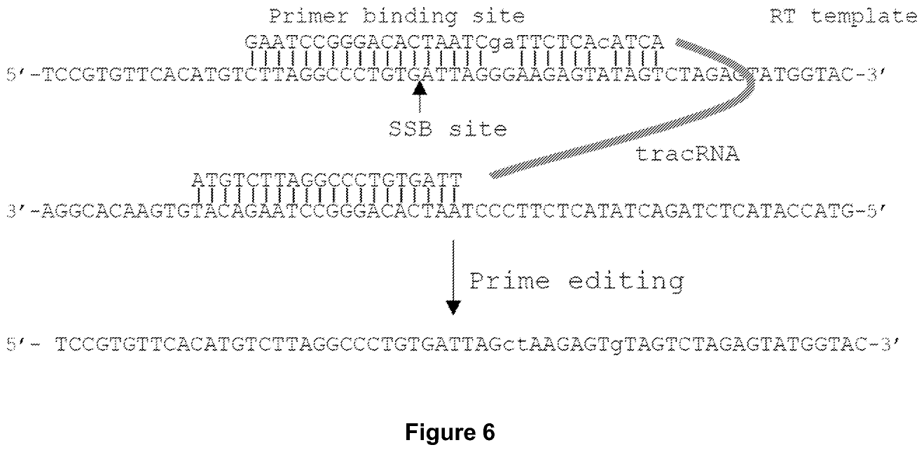

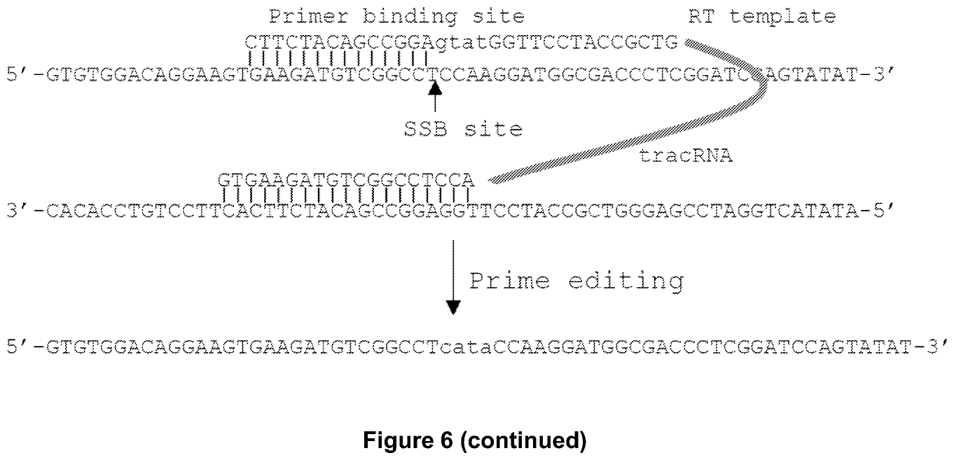

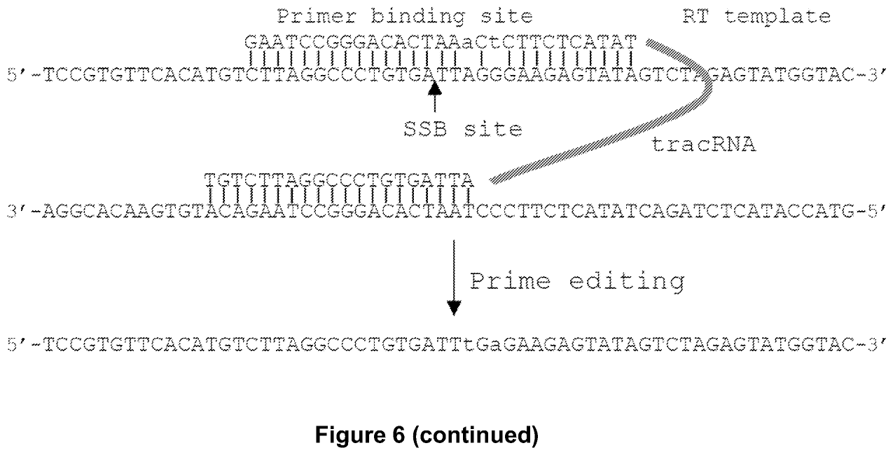

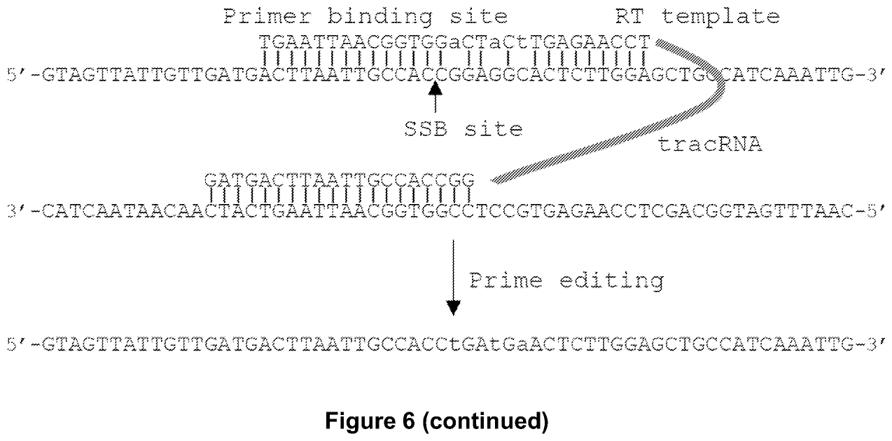

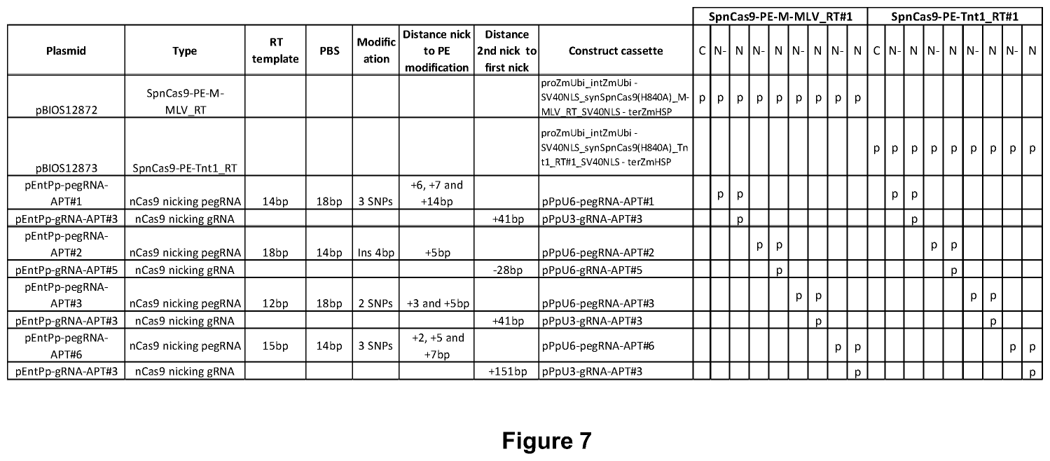

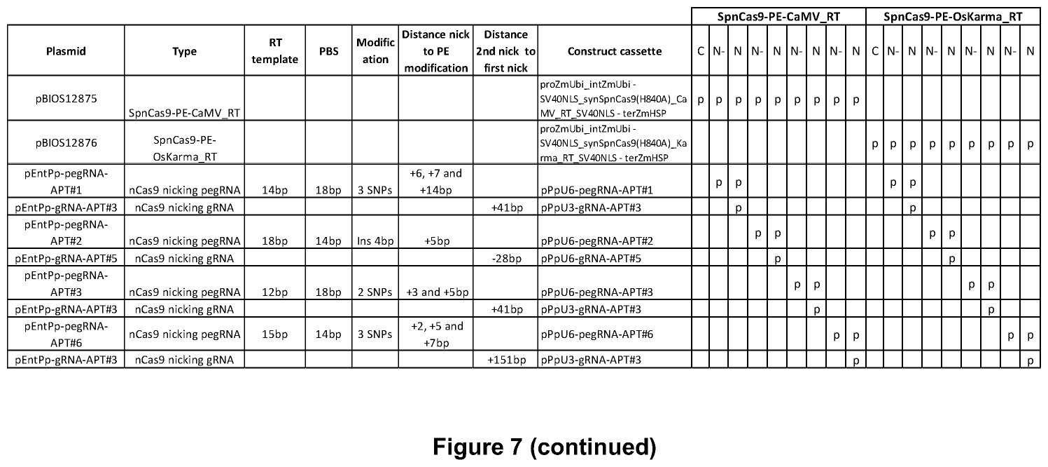

The CaMV N-terminally truncated ORF V RT domain (SEQ ID NO: 1) is fused to nCas9 (H840A) (SEQ ID NO: 39) forming nCas9-CaMV RT (SEQ ID NO: 10). Similarly, the Rice Karma RT domain (SEQ ID NO: 64) is fused to nCas9(H840A) (SEQ ID NO: 39) forming nCas9-Karma RT (SEQ ID NO: 65) or with the Tobacco TnT1 RT domain (SEQ ID NO: 7), forming nCas9-TnT1 RT (SEQ ID NO:12). nCas9-CaMV RT, nCas9-Karma RT, nCas9-TnT1 RT are nCas9-plantRT. PlantRT is a Reverse Transcriptase adapted for plants. DNA sequences of nCas9-plantRT and an nCas9-MMLV RT (PE2 editor) (SEQ ID NO: 40) were cloned in high copy number plasmids between the maize Ubiquitin promoter (SEQ ID NO: 24) and a maize HSP terminator (SEQ ID NO: 66) forming plasmids pBIOS12872 (nCas9-MMLV-RT; SEQ ID NO: 67), pBIOS12875 (nCas9-CaMV-RT; SEQ ID NO: 68), pBIOS12876 (nCas9-Karma-RT; SEQ ID NO: 69) and pBIOS12873 (nCas9_Tnt1-RT; SEQ ID NO: 70). 4 pegRNAs (pegRNA-APT-#1, #2, #3 and #6; SEQ ID NO: 72, SEQ ID NO: 74, SEQ ID NO: 76, SEQ ID NO: 78; and the respective targeted sequences SEQ ID NO: 71, SEQ ID NO: 73, SEQ ID NO: 75, SEQ ID NO: 77) were designed to introduce loss of function mutations in APT (FIGS. 6 and 7). These pegRNAs were cloned such that they are expressed from a P. patens U6 promoter (SEQ ID NO: 79). gRNAs were also designed to test the effect of adding a second DNA nick on the strand opposite to that edited by the nCas9-RT (PE3 strategy). The gRNA (gRNA-APT #3 SEQ ID NO: 81; target sequence SEQ ID NO: 80) was expressed from a P. patens U3 promoter (SEQ ID NO: 82) and gRNA-APT #5 (SEQ ID NO: 84, target sequence SEQ ID NO: 83) from the P. patens U6 promoter.

Combinations of nCas9-RT, pegRNAs and nicking gRNAs as outlined in FIG. 7 are transformed into P. patens protoplasts. The number of plants developing on 2-FA containing media is recorded and is a measure of nCas9-RT induced prime editing levels.

### Example 3: Improvements to Prime Editors with Plant-Expressed RT Domains

The MMLV-RT domain used in example 2 is a version that contains several mutations in order to optimize its activity (Anzalone et al., 2019). One optimization to MMLV-RT is the inhibition of RNaseH activity from the MMLV-RT domain. This can be achieved by expressing a C-terminal truncated version of the plant-RT that removes the entire RNaseH domain (Kotewicz et al., 1988).

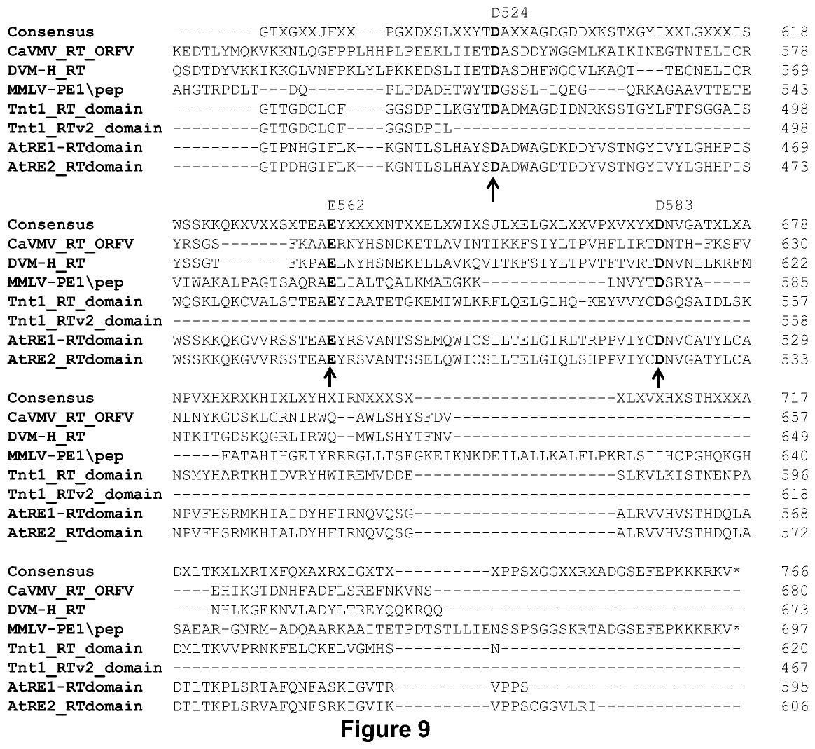

For example, a C-terminally truncated Tnt1-RT domain (protein Tnt-RTv2; SEQ ID NO: 85) was cloned between the maize Ubiquitin promoter and maize HSP terminator forming plasmid pBIOS12874 (nCas9_Tnt1-RTv2 SEQ ID NO: 86). Alternatively, amino acids that are important for RNaseH activity have been identified in RNaseH domain. Mutations in these amino acids can reduce or prevent RNaseH activity in MMLV-RT. Known mutations in MMLV-RT that reduce RNaseH activity include mutations at positions D524, S526, D583, Y586 and D653 (Blain and Goff (1993), WO2009125006A2). Equivalent residues in plant RTs can be identified by homology and sequence structure predictions (FIG. 9). Accordingly, a G to A mutation was introduced into pBIOS12873 to convert Aspartic acid 469 in the Tnt1-RT domain to Asparagine forming Tnt1(D469N) (SEQ ID NO: 87) and pBIOS12873(D469N) (nCas9-TnT1(D469N)RT SEQ ID NO: 88). This is the equivalent of the MMLV-RT D524N substitution which removes RNaseH activity. Secondly, 3 mutations were introduced into the Tnt1-RT domain again to remove RNaseH activity forming Tnt1(D524G,E512Q,D545N) (SEQ ID NO: 106). These mutations are equivalent to MMLV-RT D524G, E562Q and D583N.

A second optimization to improve Prime Editors is to adjust the length of the linker between the nCas9 and the RT domains and the flexibility of the linker. The linker used by Anzalone et al., 2019 is replaced with a longer linker used successfully in nCas9-PmCDA base-editors (Shimatani et al., 2017). This longer linker version is introduced into the initial nCas9(H840A)-Tnt1-RTv1 version forming nCas9(H840A)-SH3-Tnt1-RTv1 (SEQ ID NO: 109). Combination of an improved linker and a reduction of RT RNaseH activity is also possible.

These modified nCas9-Tnt1 versions are transformed into P. patens protoplasts together with pegRNAs and nicking gRNAs as described in example 2. The number of plants developing on 2-FA containing media is recorded and is a measure of nCas9-RT induced prime editing levels.

### Example 4: Activity of Prime Editors with Plant-Expressed RT Domains in Plant Protoplasts

A mutation changing Tyrosine 67 to a Histidine in GFP changes the fluorescence spectrum of GFP such that it moves from green to blue forming a Blue Fluorescent protein (BFP). Zong et al., (2017) made a A to G base change in a BFP gene at 218 bp (altering Serine 73 to Glycine) creating a Cas9 NGG PAM site and forming BFPm. This added Cas9 PAM allows the positioning of a gRNA in the BFPm sequence permitting an nCas9-RT and pegRNA to revert the His CAC codon to the Tyr TAC codon. This BFPm gene was used to optimize nCas9-BE performance in rice and wheat protoplasts (Zong et al., (2017)). A BFPm gene was further modified by the change of sequence CG at 183-184 bp to TT to form a Cas12a PAM (TTTV). The insertion of the Cas12a PAM change also causes a change of Valine 62 to Leucine. This remodified BFPm (BFPmm) (SEQ ID NO: 89 encoding SEQ ID NO: 90) can thus be edited by Cas9 to restore green fluorescence. As a control to ensure that the amino acid change V62L does not affect fluorescence a version of BFPmm but with the His67 restored to Tyr67 (ie the desired editing event) was synthesized (GFPmm SEQ ID NO: 91 encoding for SEQ ID NO: 92). Both BFPmm and GFPmm were linked to the strong constitutive Maize ubiquitin promoter and transformed into maize and wheat protoplasts using a standard PEG-method (Wolter et al. 2017). Only GFPmm-transformed protoplasts exhibited green fluorescence.

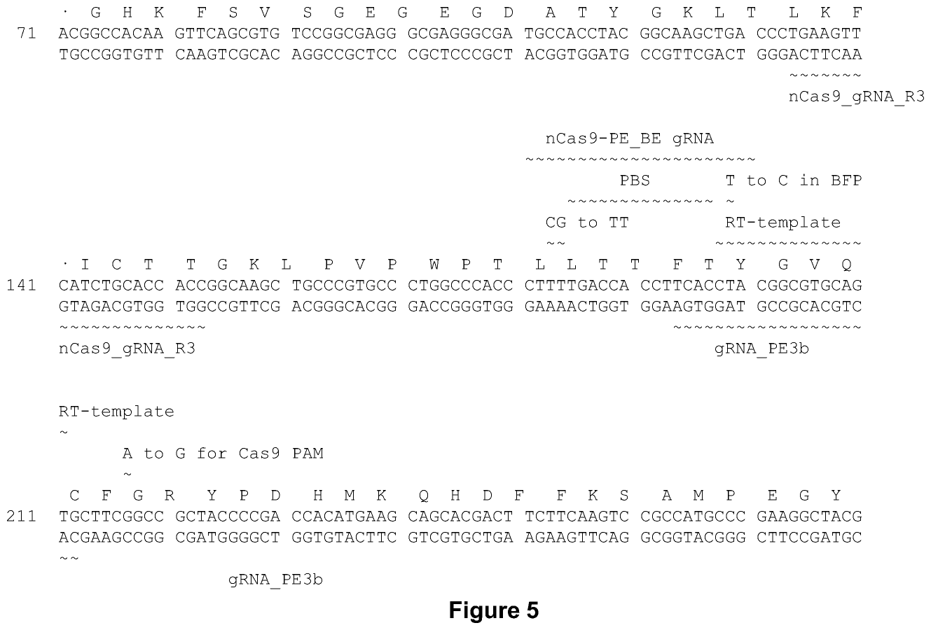

A pegRNA and two nicking gRNAs were designed to target the BFPmm gene. These pegRNA-BFP-01 (SEQ ID NO: 94, target SEQ ID NO: 93) contains a G to A change in the RT template so as to convert C to T and thus His67 to Tyr67 (FIG. 5). Three prime editing strategies are tested with the nCas9-RT versions of example 2 and 3. First the PE2 strategy without a second DNA nick; PE3 using gRNA-BFP-R3 (SEQ ID NO: 96, target SEQ ID NO: 95) creating a DNA nick on the non-edited strand −63 bp from the targeted base pair change and PE3b using gRNA-BFP-PE3b (SEQ ID NO: 98, target SEQ ID NO: 97) creating a nick −3 bp from the targeted base pair change on the non-edited strand, the gRNA targeting the edited sequence.

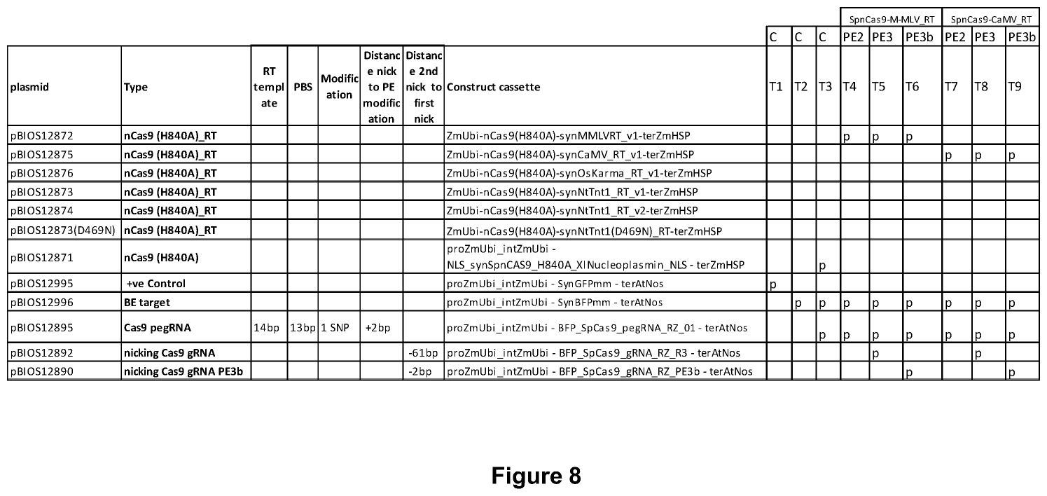

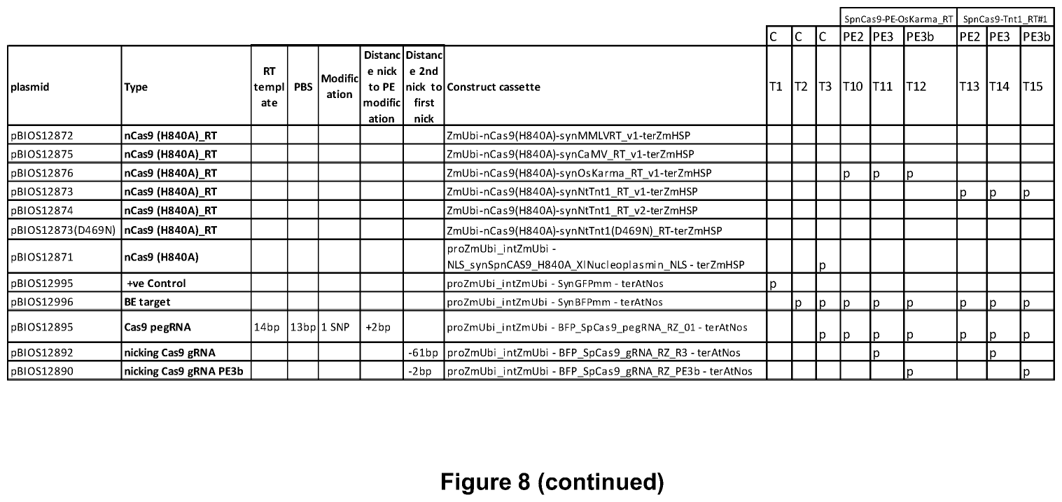

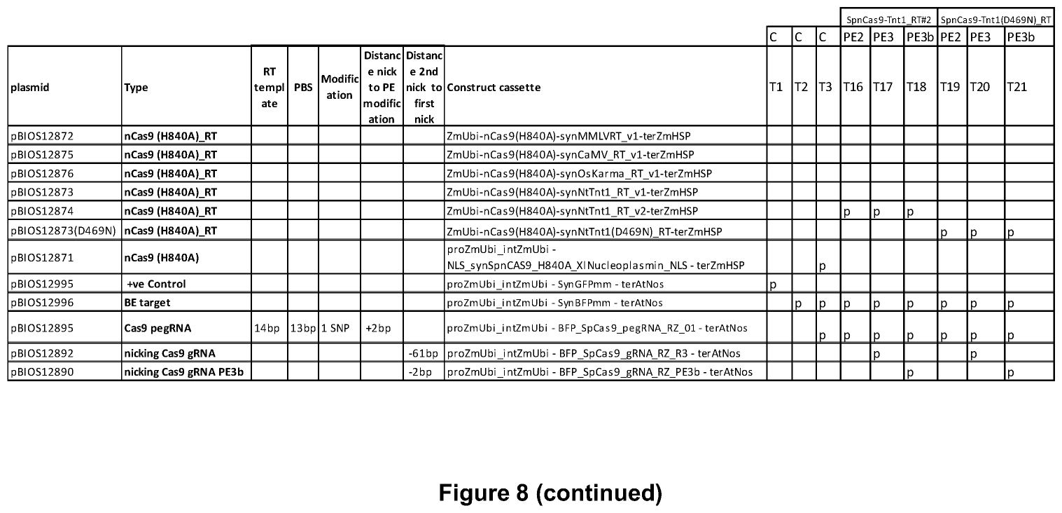

The pegRNA and two gRNAs were cloned individually between hammerhead and HDV ribozymes and then cloned between the maize ubiquitin promoter (SEQ ID NO: 24) and nos polyadenylation sequence (SEQ ID NO: 25) forming plasmids pBIOS12895 (BFP_SpCas9_pegRNA_RZ_01; SEQ ID NO: 99), pBIOS12892 (BFP_SpCas9_gRNA_RZ_R3; SEQ ID NO: 100) and pBIOS12890 (BFP_SpCas9_gRNA_RZ_PE3b; SEQ ID NO: 101). FIG. 8 shows the combinations of plasmids for transformation into wheat and into maize protoplasts. The proportion of green-fluorescent protoplasts (prime editing events) is determined 24 h to 48 h after transformations.

### Example 5: Activity of Prime Editors with Plant-Expressed RT Domains in Maize

These nCas9-plantRT (SEQ ID NO: 10-12-65-104-105-107-109) and the PE2 editor (SEQ ID NO: 40) are tested for Prime Editing activity in maize. The chosen target is the herbicide and selectable marker gene acetohydroxyacid synthase (AHAS or ALS). Mutations Pro-165-Ala or Ser-621-Asn in ALS genes lead to resistance of maize callus to sulfonylurea herbicides such as chlorosulfuron or imazethapyr (Zhu et al. 1999). Maize has two ALS genes ALS1 (SEQ ID NO: 13) and ALS2 (SEQ ID NO: 14). pegRNAs are designed to introduce the Pro-165-Ser or Ser-621-Asn mutations into ZmALS2.

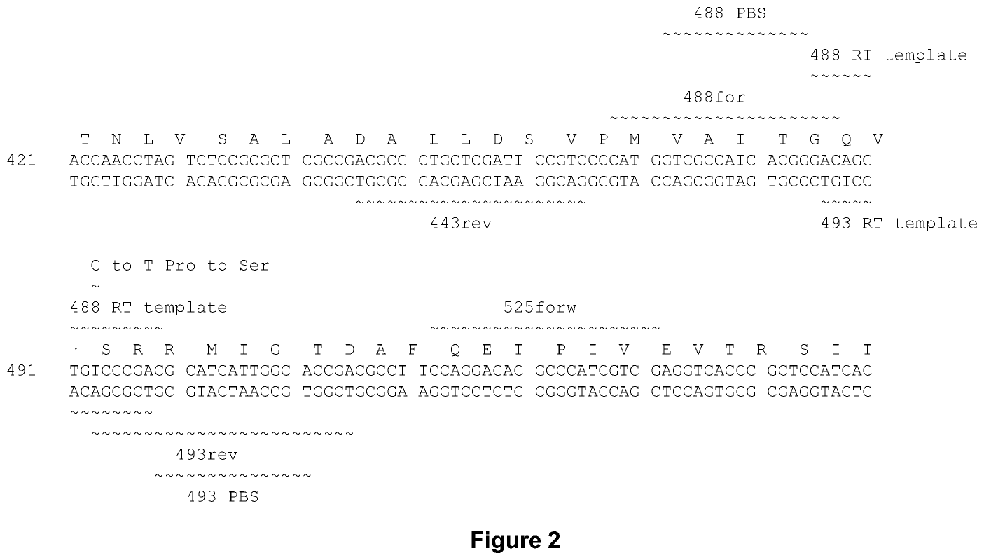

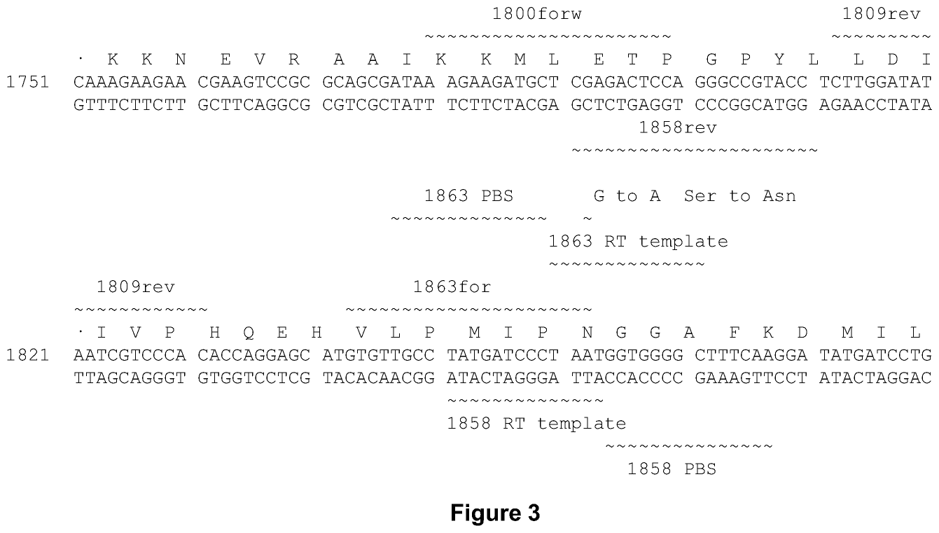

Two sets of pegRNAs and an associated guide to nick the non-edited strand are designed per target as shown in FIGS. 2 and 3 and table 1. The pegRNAs and gRNAs are cloned into high copy number plasmids behind a maize U6 promoter (SEQ ID NO: 15) (cassettes SEQ ID NO: 16-23). The nCas9-plantRT and the PE2 editor nucleic acids are cloned in high copy number plasmids between the maize Ubiquitin promoter (SEQ ID NO: 24) and an HSP terminator (SEQ ID NO: 66).

TABLE 1
Components of pegRNAs and gRNAs used to modify ZmALS2

| | | | | | | RT- | |
|---|---|---|---|---|---|---|---|
| Type | name | Gene | Target | Spacer | Scaffold | template | PBS |
| pegRNA | 488forw | ZmALS2 | Pro165 | SEQ ID | Scaffold | Gtcgcgaca | SEQ ID |
| | | | to Ser | NO: 41 | | cctgtc SEQ | NO: 43 |
| | | | | | | ID NO: 42 | |
| pegRNA | 493rev | ZmALS2 | Pro165 | SEQ ID | Scaffold | acaggtgtcg | SEQ ID |
| | | | to Ser | NO: 44 | | cga SEQ | NO: 46 |
| | | | | | | ID NO: 45 | |
| pegRNA | 1858rev | ZmALS2 | Ser621 | SEQ ID | Scaffold | tatgatcccta | SEQ ID |
| | | | to Asn | NO: 47 | | at SEQ ID | NO: 49 |
| | | | | | | NO: 48 | |
| pegRNA | 1863forw | ZmALS2 | Ser621 | SEQ ID | Scaffold | gccccaccat | SEQ ID |
| | | | to Asn | NO: 50 | | ta SEQ ID | NO: 52 |
| | | | | | | NO: 51 | |
| gRNA | 443rev | ZmALS2 | Pro165 | SEQ ID | Scaffold | | |
| | | | to Ser | NO: 53 | | | |
| gRNA | 525forw | ZmALS2 | Pro165 | SEQ ID | Scaffold | | |
| | | | to Ser | NO: 54 | | | |
| gRNA | 1800forw | ZmALS2 | Ser621 | SEQ ID | Scaffold | | |
| | | | to Asn | NO: 55 | | | |
| gRNA | 1809rev | ZmALS2 | Ser621 | SEQ ID | Scaffold | | |
| | | | to Asn | NO: 56 | | | |

The underlined nucleotides indicate the edit that is to be added in the target sequence.

Each pegRNA plus gRNA is co-transformed with PE2 editor or a nCas9-plantRT into maize A188 protoplasts using a standard PEG-based protocol. The Pro-165-Ser target site in ZmALS2 is amplified using primers ZmALS_165_for (SEQ ID NO: 26) and ZmALS_165_rev (SEQ ID NO: 27) and the Ser-621-Asn target site is amplified using primers ZmALS_621_for (SEQ ID NO: 28) and ZmALS_621_rev (SEQ ID NO: 29). Amplicons are sequenced using Next Generation Sequencing (NGS) technology. The number of sequences with the desired C to T (Pro-165-Ala) or G to A (Ser-621-Asn) edit is assessed to determine the relative efficiency of PE2 editor versus various nCas9-PlantRT.

Each pegRNA plus gRNA is co-bombarded with PE2 or a nCas9-plantRT into maize BMS callus using a standard biolistic protocol. The transformed callus is selected on chlorosulfuron as described in Zhu 1999. The target site in ZmALS2 is amplified from DNA from chlorosulfuron-resistant calli using primers ZmALS_165_for (SEQ ID NO: 26) and ZmALS_165_rev (SEQ ID NO: 27) or primers ZmALS_621_for (SEQ ID NO: 28) and ZmALS_621_rev (SEQ ID NO: 29). Amplicons are sequenced using NGS. The number of sequences with the desired C to T (Pro-165-Ala) or G to A (Ser-621-Asn) edit is assessed to determine the relative efficiency of PE2 editor versus various nCas9-PlantRT.

### Example 6: Activity of Prime Editors with Plant-Expressed RT Domains in Wheat

PE2 editor and the nCas9-plantRT described in example 2 and 3 (nCas9-CaMV RT, nCas9-TnT1 RT, nCas9-Tnt1(D469N)-RT, nCas9-Tnt1v2-RT and nCas9-Karma RT, nCas9-Tnt1(D469G, E512Q, D545N)-RT, nCas9-SH3-Tnt1-RT) are tested in wheat by targeting the wheat ACCase gene. A mutation at amino acid 2004 changing Alanine to Valine gives resistance to the herbicide quizalofop (Ostlie et al. 2015). The sequences of the targeted exon in genomes A, B and D of wheat variety Fielder are SEQ ID NO: 30-32.

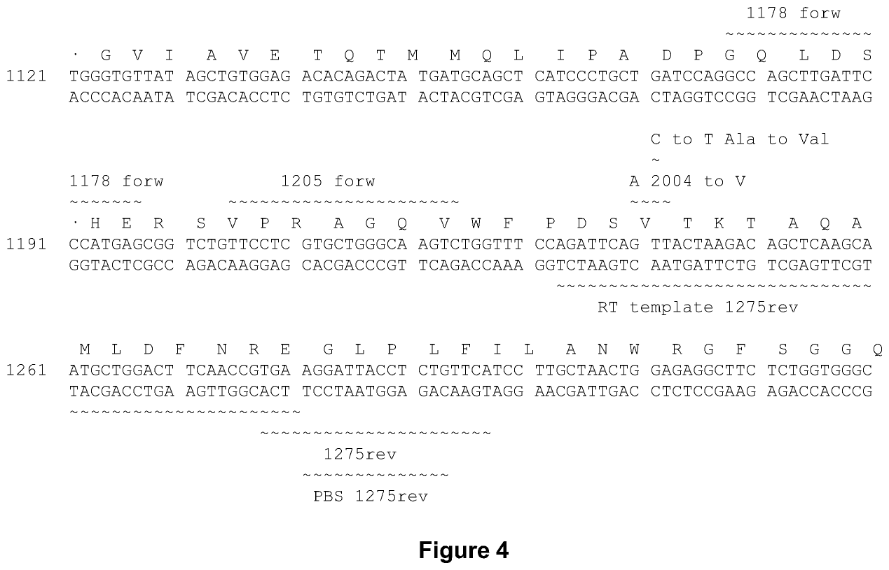

A pegRNA is designed to create this change in the wheat variety Fielder in genomes A, B and D together with associated guides to nick the non-edited strand (FIG. 4 and table 2). The pegRNAs and gRNAs are cloned into high copy number plasmids behind a Wheat U6 promoter (SEQ ID NO: 33) (cassettes SEQ ID NO: 34-36). PE2 editor and the nCas9-plantRT nucleic acids are cloned in high copy number plasmids between the maize Ubiquitin promoter (SEQ ID NO: 24) and an HSP terminator (SEQ ID NO: 66).

TABLE 2
Components of pegRNA and gRNAs used to modify TaACCase

| Type | name | Gene | Target | Spacer | Scaffold | RT-template | PBS |
|---|---|---|---|---|---|---|---|
| pegRNA | 1275forw | TaACCase | Ala | SEQ ID | Scaffold | agattcagttactaag | SEQ ID |
| | | | 2004 | NO: 57 | | acagctcaagcaatg | NO: 59 |
| | | | to Val | | | ctggacttcaaccgtg | |
| | | | | | | a SEQ ID NO: 58 | |
| gRNA | 1178forw | TaACCase | Ala | SEQ ID | Scaffold | | |
| | | | 2004 | NO: 60 | | | |
| | | | to Val | | | | |
| gRNA | 1205forw | TaACCase | Ala | SEQ ID | Scaffold | | |
| | | | 2004 | NO: 61 | | | |
| | | | to Val | | | | |

The pegRNA plus one or both gRNAs are co-transformed with PE2 editor or a nCas9-plantRT into wheat Fielder protoplasts using a standard PEG-based protocol. The Ala-2004-Val target site in TaACCase is amplified using primers TaACCase_forw (SEQ ID NO: 37) and TaACCase_rev (SEQ ID NO: 38). Amplicons are sequenced using Next Generation Sequencing (NGS) technology. The number of sequences with the desired C to T (Ala-2004-Val) edit is assessed to determine the relative efficiency of PE2 editor versus various nCas9-PlantRT.

TABLE 3
Description of sequences

| Sequence number | Name of the sequence |
|---|---|
| SEQ ID NO: 1 | CaVMV_RT_ORFV_delNterm |
| SEQ ID NO: 2 | DVM-H_Rtdomain |
| SEQ ID NO: 3 | AtRE1-RTdomain |
| SEQ ID NO: 4 | AtRE2_RTdomain |
| SEQ ID NO: 5 | OsLullaby_chr2_RTdomain |
| SEQ ID NO: 6 | OsRIRE7_RTdomain |
| SEQ ID NO: 7 | Tnt1_RT_domain |
| SEQ ID NO: 8 | DmCopia-drosophila_Rtdomain |
| SEQ ID NO: 9 | M-MLV_RTmut_PE2 |
| SEQ ID NO: 10 | nCas9-CaMVRT |
| SEQ ID NO: 11 | nCas9-LullabyRT |
| SEQ ID NO: 12 | nCas9-TnT1RT |
| SEQ ID NO: 13 | ZmALS1 coding sequence |
| SEQ ID NO: 14 | ZmALS2 coding sequence |
| SEQ ID NO: 15 | ZmU6 promoter |
| SEQ ID NO: 16 | ZmU6_pegRNA_488forw |
| SEQ ID NO: 17 | ZmU6_pegRNA_493rev |
| SEQ ID NO: 18 | ZmU6_pegRNA_1858rev |
| SEQ ID NO: 19 | ZmU6_pegRNA_1863forw |
| SEQ ID NO: 20 | ZmU6_gRNA_443rev |
| SEQ ID NO: 21 | ZmU6_gRNA_542forw |
| SEQ ID NO: 22 | ZmU6_gRNA_1800forw |
| SEQ ID NO: 23 | ZmU6_gRNA_1809rev |
| SEQ ID NO: 24 | ZmUbiquitin promoter + 5′UTR |
| SEQ ID NO: 25 | Nopaline synthase polyadenylation sequence |
| SEQ ID NO: 26 | ZmALS_165_for |
| SEQ ID NO: 27 | ZmALS_165_rev |
| SEQ ID NO: 28 | ZmALS_621_for |
| SEQ ID NO: 29 | ZmALS_621_rev |
| SEQ ID NO: 30 | TaACCase target exon genome A |
| SEQ ID NO: 31 | TaACCase target exon genome B |
| SEQ ID NO: 32 | TaACCase target exon genome D |
| SEQ ID NO: 33 | TaU6 promoter |
| SEQ ID NO: 34 | TaU6_pegRNA_1275rev |
| SEQ ID NO: 35 | TaU6_gRNA_1178forw |
| SEQ ID NO: 36 | TaU6_pegRNA_1205forw |
| SEQ ID NO: 37 | TaACCase_forw |
| SEQ ID NO: 38 | TaACCase_rev |
| SEQ ID NO: 39 | nCas9(H840A) |
| SEQ ID NO: 40 | PE2 editor |

### REFERENCES

Anzalone A V, Randolph P B, Davis J R, Sousa A A, Koblan L W, Levy J M, Chen P J, Wilson C, Newby G A, Raguram A, Liu D R. 2019. Search-and-replace genome editing without double-strand breaks or donor DNA. Nature. 2019 Oct. 21

Blain, S. W., and Goff, S. P. (1993). Nuclease Activities of Moloney Murine Leukemia Virus Reverse Transcriptase. J. Biol. Chem. 268, 23585-23592.

Christensen, A. H., Quail, P. H. Ubiquitin promoter-based vectors for high-level expression of selectable and/or screenable marker genes in monocotyledonous plants. Transgenic Research 5, 213-218 (1996) https://doi.org/10.1007/BF01969712

Depigny-This, D., Raynal, M., Aspart, L. et al. The cruciferin gene family in radish. Plant Mol Biol 20, 467-479 (1992). https://doi.org/10.1007/BF00040606

Hess G T, Frésard L, Han K, et al. Directed evolution using dCas9-targeted somatic hypermutation in mammalian cells. Nat Methods. 2016;13(12):1036-1042. doi:10.1038/nmeth.4038

Ishida et al., Nat. Biotechnol., 14:745-750, 1996

Kay R, Chan A, Daly M, McPherson J. Duplication of CaMV 35S Promoter Sequences Creates a Strong Enhancer for Plant Genes. Science. 1987 Jun 5;236(4806):1299-302. doi: 10.1126/science.236.4806.1299. PMID: 17770331.

Kotewicz, M. L., Sampson, C. M., D'Alessio, J. M. and Gerard, G. F. (1988). Isolation of cloned Moloney murine leukemia virus reverse transcriptase lacking ribonuclease H activity. Nucleic Acids Res., 16, 265-277.

Liu. CRISPR Meeting CSHL 2019 Oct 10-13

McElroy, Zhang, Cao, Wu, Isolation of an efficient actin promoter for use in rice transformation, The Plant Cell Feb 1990, 2 (2) 163-171; DOI: 10.1105/tpc.2.2.163

Ming, M., Ren, Q., Pan, C. et al. CRISPR-Cas12b enables efficient plant genome engineering. Nat. Plants 6, 202-208 (2020). https://doi.org/10.1038/s41477-020-0614-6

Neumann P, Novák P, Hos̆táková N, Macas J. (2019). Systematic survey of plant LTR-retrotransposons elucidates phylogenetic relationships of their polyprotein domains and provides a reference for element classification. Mol DNA. 10:1. doi: 10.1186/s13100-018-0144-1.

Ostlie M, Haley SD, Anderson V, Shaner D, Manmathan H, Beil C and Westra P (2015) Development and Characterization of Mutant Winter Wheat (Triticum Aestivum L.) Accessions Resistant to the Herbicide Quizalofop. Theor Appl Genet, 128 (2), 343-51.

Shimatani Z, Kashojiya S, Takayama M, Terada R, Arazoe T, Ishii H, Teramura H, Yamamoto T, Komatsu H, Miura K, Ezura H, Nishida K, Ariizumi T, Kondo A. (2017). Targeted base editing in rice and tomato using a CRISPR-Cas9 cytidine deaminase fusion. Nat Biotechnol.; 35(5):441-443. doi: 10.1038/nbt.3833.

Takatsuji, H., H. Hirochika, T. Fukushi, and J. E. Ikeda. 1986. Expression of cauliflower mosaic virus reverse transcriptase in yeast. Nature 319:240-243.

Takatsuji H., Yamauchi H., Watanabe S., Kato H., Ikeda J E. Cauliflower mosaic virus reverse transcriptase. Activation by proteolytic processing and functional alteration by terminal deletion. J Biol Chem. 1992 Jun 5; 267(16):11579-85.

Trouiller et al. (2006) MSH2 is essential for the preservation of genome integrity and prevents homeologous recombination in the moss Physcomitrella patens. Nucleic Acids Res. 34:232-42. doi: 10.1093/nar/gkj423.

Verdaguer, B., de Kochko, A., Fux, C. I. et al. Functional organization of the cassava vein mosaic virus (CsVMV) promoter. Plant Mol Biol 37, 1055-1067 (1998). https://doi.org/10.1023/A:1006004819398

Wolter et al. Characterization of paired Cas9 nickases induced mutations in maize mesophyll protoplasts. Maydica Vol 62, No 2 (2017).

You et al. (2012), Facilitated Substrate Channeling in a Self-Assembled Trifunctional Enzyme Complex. Angew. Chem. Int. Ed., 51: 8787-8790.

Zetsche B, Gootenberg J S, Abudayyeh O O, et al. Cpf1 is a single RNA-guided endonuclease of a class 2 CRISPR-Cas system. Cell. 2015; 163(3):759-771. doi:10.1016/j.cell.2015.09.038

Zetsche et al. (2015) A split-Cas9 architecture for inducible genome editing and transcription modulation. Nat. Biotech. 33:139-42. doi: 10.1038/nbt.3149.)

Zhu T, Peterson D J, Tagliani L, St Clair G, Baszczynski C L and Bowen B (1999). Targeted manipulation of maize genes in vivo using chimeric RNA/DNA oligonucleotides. Proc Natl Acad Sci USA. 96(15):8768-73.

Zong et al. (2017). Precise base editing in rice, wheat and maize with a Cas9-cytidine deaminase fusion. Nat Biotechnol. 35:438-440. doi: 10.1038/nbt.3811.

1. A method for inserting a desired edit at a target site in a double-stranded DNA sequence in a plant cell comprising:
(a) providing a Cas nickase associated with a reverse transcriptase adapted to plants and a prime-editing guide RNA (pegRNA) to the double-stranded DNA sequence, wherein the pegRNA comprises (from 5′ to 3′):
(i) a single guide RNA region which hybridizes to a DNA strand at the target site, thereby directing the Cas nickase associated with the reverse transcriptase to the target site,
(ii) a guide scaffold sequence which allows Cas-binding,
(iii) a template RNA containing the desired edit, which serves as a template for creating the edited DNA strand, and
(iv) a primer binding site (PBS) that allows the 3′ end of the nicked DNA strand to hybridize to the pegRNA, and serve as a reverse transcriptase to start reverse transcription;
(b) wherein one strand of the double-stranded DNA sequence is cut by the nickase, thereby generating a free single-strand DNA (ssDNA) having a 3′ end;
(c) wherein the 3′ end of the free ssDNA hybridizes to the PBS of the pegRNA,
(d) wherein the reverse transcriptase performs reverse transcription of the template RNA of the pegRNA and elongates the 3′ extremity of the ssDNA,
(e) thereby generating a ssDNA flap comprising the desired edit and is complementary to the DNA synthesis template;
(f) wherein an endogenous DNA strand adjacent to the cut site is replaced with the ssDNA flap, thereby installing the desired edit at the target site in the double-stranded DNA sequence.

2. The method of claim 1, wherein the plant cell is present in a plant tissue or in a whole plant.

3. A method for obtaining a plant having a desired edit at a target site comprising:
(a) performing the method of claim 1 to a plant cell or plant tissue comprising in its genome a sequence of interest containing the target site,
(b) culturing the plant cell or plant tissue in adequate conditions for multiplication of cells, and
(c) growing a plant from the cultured plant cell(s) or plant tissues.

4. The method of claim 3, further comprising after (b) and before (c), screening the cultured plant cell(s) or plant tissue(s) from (b) to identify cells containing the desired edit introduced at the target site and isolating such cells, wherein the plant is grown in (c) if the screening indicated that the desired edit was introduced at the target site.

5. The method of claim 1, wherein the Cas nickase is a mutated Cas9 protein.

6. The method of claim 1, wherein the reverse transcriptase is selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 64, SEQ ID NO: 85, SEQ ID NO: 87, and SEQ ID NO: 106.

7. The method of claim 1, wherein the Cas nickase protein is fused with the reverse transcriptase adapted to plants.

8. The method of claim 1, further comprising delivering a supplementary single guide RNA (gRNA) targeting a secondary site in a vicinity of the target site and introducing, a Cas nickase to introduce a nick in a DNA sequence at the secondary site.

9. The method of claim 1, wherein the plant is a monocotyledon.

10. A vector comprising a DNA construct coding for a Cas nickase and a DNA construct coding a reverse transcriptase adapted to plants, with genetic elements allowing transcription in a plant cell.

11. The vector of claim 10, wherein the DNA construct is coding for a Cas nickase fused with a reverse transcriptase adapted to plants

12. A fusion protein comprising a Cas nickase fused with a reverse transcriptase adapted to plants, or
a complex comprising a Cas nickase associated by binding domains with a reverse transcriptase adapted to plants,
wherein the reverse transcriptase adapted to plants preferably comprises SEQ ID NO: 85, SEQ ID NO: 87, or SEQ ID NO: 106.

13. A kit to perform the method of claim 1, comprising one or more vectors, wherein the one or more vectors comprise(s) a sequence coding for a Cas nickase, a sequence coding for a reverse transcriptase adapted to plants, and a sequence transcribed to the pegRNA.

14. The kit of claim 13, comprising two vectors, wherein one of the vectors comprises a sequence coding for the Cas nickase and a sequence coding for the reverse transcriptase adapted to plants, and the other vector contains the sequence transcribed to the pegRNA.

15. The kit of claim 13 comprising one vector, which contains a sequence coding for the nickase, a sequence coding for the reverse transcriptase adapted to plants, and the sequence transcribed to the pegRNA.

16. (canceled)

17. (canceled)

18. A plant comprising in its genome or in an extrachromosomal vector the DNA construct of claim 10.

19. The plant of claim 18, further comprising in its genome or in an extrachromosomal vector and a pegRNA guide comprising (from 5′ to 3′):
(i) a single guide RNA region which hybridizes to a DNA strand at the target site, thereby directing the Cas nickase associated with the reverse transcriptase to the site,
(ii) a guide scaffold sequence which allows Cas-binding,
(iii) a template RNA containing the desired edit, which serves as a template for creating the edited DNA strand, and
(iv) a primer binding site (PBS) that allows the 3′ end of the nicked DNA strand to hybridize to the pegRNA, and serve as a reverse transcriptase to start reverse transcription,
wherein the single guide RNA region hybridizes to a DNA strand at a target site, and a template RNA contains a desired edit to be performed at the target site.

20-24. (canceled)

25. The method of claim 9, wherein the monocotyledon is a cereal.

26. The kit of claim 14, wherein sequence coding for the Cas nickase and the sequence coding for the reverse transcriptase adapted to plants are fused within the same gene.

27. The kit of claim 15, wherein the sequence coding for the nickase and the sequence coding for the reverse transcriptase adapted to plants are fused within the same gene.
